(19) United States
(12) Patent Application Publication
(10) Pub. No.: US 2018/0346571 A1
(43) Pub. Date: Dec. 6, 2018

(54) PD-L1-BINDING AGENTS AND USES THEREOF

(71) Applicant: Oncomed Pharmaceuticals, Inc., Redwood City, CA (US)

(72) Inventors: Austin L. GURNEY, San Francisco, CA (US); Ming-Hong XIE, Foster City, CA (US)

(21) Appl. No.: 15/777,175

(22) PCT Filed: Nov. 16, 2016
(86) PCT No.: PCT/US2016/062320

§ 371 Date: May 17, 2018

Related U.S. Application Data
(60) Provisional application No. 62/256,256, filed on Nov. 17, 2015.

(51) Int. Cl. C07K 16/28; C07K 16/46; C07K 16/30; A61K 47/68; A61K 45/06; A61K 39/395; A61P 35/00

(52) U.S. Cl. CPC C07K 16/2827; C07K 16/468; C07K 16/30; A61K 47/6879; A61K 45/06; A61K 2039/505; A61P 35/00; C07K 2317/24; C07K 2317/21; C07K 2317/31; C07K 2317/526; A61K 39/3955

(57) ABSTRACT

Agents that specifically bind PD-L1 are disclosed. The PD-L1-binding agents may include polypeptides, antibodies, bispecific agents, homodimeric molecules, and/or heterodimeric molecules. Also disclosed are methods of using the agents for enhancing the immune response and/or treatment of diseases such as cancer.

### CROSS-REFERENCE TO RELATED APPLICATION

This application claims the priority benefit of U.S. Provisional Application No. 62/256,256, filed Nov. 17, 2015, which is hereby incorporated by reference herein in its entirety.

### INCORPORATION BY REFERENCE

The content of the following submission on ASCII text file is incorporated herein by reference in its entirety: a computer readable form (CRF) of the Sequence Listing (file name: 51760-501001WOSEQLIST.txt, date recorded: Nov. 16, 2016, size: 60,037 bytes).

### FIELD OF THE INVENTION

The present invention generally relates to agents that bind human PD-L1, particularly antibodies and bispecific agents that specifically bind the extracellular domain of PD-L1, as well as to methods of using the agents for the modulation of immune responses and/or the treatment of diseases such as cancer.

### BACKGROUND OF THE INVENTION

The basis for immunotherapy is the manipulation and/or modulation of the immune system, including both innate immune responses and adaptive immune responses. The general aim of immunotherapy is to treat diseases by controlling the immune response to a “foreign agent”, for example a pathogen or a tumor cell. However, in some instances immunotherapy is used to treat autoimmune diseases which may arise from an abnormal immune response against proteins, molecules, and/or tissues normally present in the body Immunotherapy may include agents and methods to induce or enhance specific immune responses or to inhibit or reduce specific immune responses.

The immune system is a highly complex system made up of a great number of cell types, including but not limited to, T-cells, B-cells, natural killer cells, antigen-presenting cells, dendritic cells, monocytes, and macrophages. These cells possess complex and subtle systems for controlling their interactions and responses. The cells utilize both activating and inhibitory mechanisms and feedback loops to keep responses in check and not allow negative consequences of an uncontrolled immune response (e.g., autoimmune diseases).

The concept of cancer immunosurveillance is based on the theory that the immune system can recognize tumor cells, mount an immune response, and suppress the development and/or progression of a tumor. However, it is clear that many cancerous cells have developed mechanisms to evade the immune system which can allow for uninhibited growth of tumors. Cancer/tumor immunotherapy focuses on the development of new and novel agents that can activate and/or boost the immune system to achieve a more effective attack against tumor cells resulting in increased killing of tumor cells and/or inhibition of tumor growth.

### BRIEF SUMMARY OF THE INVENTION

The present invention provides agents that bind programmed cell death ligand 1 (PD-L1), including antibodies and bispecific agents that specifically bind the extracellular domain of PD-L1. In certain embodiments, the agent is a PD-L1 or PD-1 antagonist. The invention provides methods of using the agents. In some embodiments, the invention provides methods of using the agents for immunotherapy. In some embodiments, the invention provides methods of using the agents for cancer immunotherapy. In some embodiments, the agents are used in methods of inducing, activating, promoting, increasing, enhancing, or prolonging an immune response. In some embodiments, the agents are used in methods of inducing, activating, promoting, increasing, enhancing, or prolonging an immune response to cancer and/or a tumor. In some embodiments, the agents are used in methods of inhibiting the growth of a tumor or tumor cells. In some embodiments, the agents are used in methods for the treatment of cancer. In some embodiments, the methods comprise inhibiting the growth of cancer cells. In some embodiments, the agents are used in combination with at least one additional therapeutic agent.

The invention also provides compositions, such as pharmaceutical compositions, comprising the agents described herein. Polynucleotides and/or vectors encoding the agents and methods of making the agents are also provided. Cells comprising or producing the agents described herein are provided as well as cells comprising the polynucleotides and/or the vectors described herein.

In one aspect, the present invention provides agents that bind human PD-L1. In some embodiments, the agent is an antibody. In some embodiments, an antibody that specifically binds the extracellular domain of human PD-L1 comprises: a heavy chain CDR1 comprising TSYWMH (SEQ ID NO:4), a heavy chain CDR2 comprising AIYPGNSDTSYNQKFKG (SEQ ID NO:5), and a heavy chain CDR3 comprising WGYGFDGAMDY (SEQ ID NO:6), and/or a light chain CDR1 comprising RASQDIGSSLN (SEQ ID NO:7), a light chain CDR2 comprising ATSSLDS (SEQ ID NO:8), and a light chain CDR3 comprising LQYASSP (SEQ ID NO:9).

In some embodiments, an antibody that specifically binds the extracellular domain of PD-L1 comprises: a heavy chain variable region having at least 90% sequence identity to SEQ ID NO:10 or SEQ ID NO:14, and/or a light chain variable region having at least 90% sequence identity to SEQ ID NO:11 or SEQ ID NO:15. In some embodiments, an antibody comprises a heavy chain variable region having at least 95% sequence identity to SEQ ID NO:10 or SEQ ID NO:14, and/or a light chain variable region having at least 95% sequence identity to SEQ ID NO:11 or SEQ ID NO:15. In some embodiments, an antibody comprises a heavy chain variable region comprising SEQ ID NO:10 and a light chain variable region comprising SEQ ID NO:11. In some embodiments, an antibody comprises a heavy chain variable region comprising SEQ ID NO:14 and a light chain variable region comprising SEQ ID NO:15.

In some embodiments, an agent is a monoclonal antibody, a humanized antibody, a human antibody, a recombinant antibody, a chimeric antibody, a bispecific antibody, an antibody fragment comprising an antigen-binding site, an IgG antibody, an IgG1 antibody, an IgG2 antibody, or an IgG4 antibody.

In some embodiments, an antibody that specifically binds human PD-L1 comprises a heavy chain amino acid sequence of SEQ ID NO:17 or SEQ ID NO:19 and a light chain amino acid sequence of SEQ ID NO:21.

In some embodiments, an antibody that specifically binds human PD-L1 comprises the heavy chain variable region and the light chain variable region from antibody 332M7. In some embodiments, the antibody comprises the heavy chain variable region encoded by the plasmid deposited with ATCC as PTA-122627. In some embodiments, the antibody comprises the light chain variable region encoded by the plasmid deposited with ATCC as PTA-122628. In some embodiments, the antibody comprises the light chain encoded by the plasmid deposited with ATCC as PTA-122628. In some embodiments, the antibody comprises the heavy chain variable region encoded by the plasmid deposited with ATCC as PTA-122627 and the light chain variable region encoded by the plasmid deposited with ATCC as PTA-122628.

In some embodiments, an antibody described herein specifically binds human PD-L1 and does not bind mouse PD-L1. In some embodiments, an antibody described herein specifically binds human PD-L1 and binds mouse PD-L1 at a level that is greatly reduced as compared to the binding of the antibody to human PD-L1. In some embodiments, an antibody described herein specifically binds human PD-L1 and specifically binds cynomolgus monkey PD-L1.

In another aspect, the invention provides a plasmid deposited with ATCC and assigned designation number PTA-122627 and a plasmid deposited with ATCC and assigned designation number PTA-122628.

In some embodiments of each of the aforementioned aspects and embodiments, as well as other aspects and embodiments described herein, the agent that specifically binds PD-L1 is an antibody. In some embodiments, the antibody is monovalent. In some embodiments, the antibody is bivalent. In some embodiments, the antibody is monospecific. In some embodiments, the antibody is bispecific. In some embodiments, the antibody is part of a bispecific homodimeric molecule. In some embodiments, the antibody is part of a bispecific heterodimeric molecule.

In some embodiments, a heterodimeric molecule comprises a first arm which binds human PD-L1 and a second arm which binds a second target. In some embodiments, a heterodimeric molecule comprises a first arm that specifically binds human PD-L1 and a second arm, wherein the first arm comprises an anti-PD-L1 antibody described herein. In some embodiments, a heterodimeric molecule comprises a first arm that binds human PD-L1 and a second arm which comprises an antigen-binding site from an antibody that specifically binds a second target. In some embodiments, a heterodimeric molecule is a bispecific antibody. In some embodiments, a heterodimeric molecule comprises a first arm that binds human PD-L1 and a second arm that specifically binds a tumor antigen. In some embodiments, a heterodimeric molecule comprises a first arm that binds human PD-L1 and a second arm that specifically binds PD-1, TIGIT, CTLA-4, TIM-3, LAG-3, OX-40, 4-1BB, or GITR. In some embodiments, a heterodimeric molecule comprises a first arm that binds PD-L1 and a second arm that comprises an immunotherapeutic agent. In some embodiments, the immunotherapeutic agent is selected from the group consisting of: granulocyte-macrophage colony stimulating factor (GM-CSF), macrophage colony stimulating factor (M-CSF), granulocyte colony stimulating factor (G-CSF), interleukin 2 (IL-2), interleukin 3 (IL-3), interleukin 12 (IL-12), interleukin 15 (IL-15), B7-1 (CD80), B7-2 (CD86), 4-1BB ligand, GITRL, OX-40L, anti-CD3 antibody, anti-CTLA-4 antibody, anti-PD-1 antibody, anti-TIGIT antibody, anti-4-1BB antibody, anti-GITR antibody, anti-OX-40 antibody, anti-LAG-3 antibody, and anti-TIM-3 antibody.

In some embodiments, a heterodimeric molecule described herein comprises a first arm comprising a first CH3 domain and a second arm comprising a second CH3 domain wherein each CH3 domain is modified to promote formation of heterodimers. In some embodiments, the CH3 domains are modified based upon electrostatic effects. In some embodiments, the CH3 domains are modified using a knobs-into-holes technique.

In some embodiments of each of the aforementioned aspects and embodiments, as well as other aspects and embodiments described herein, an agent described herein specifically binds human PD-L1 and inhibits binding of PD-L1 to PD-1. In some embodiments, an agent specifically binds PD-L1 and inhibits or blocks the interaction between PD-L1 and PD-1. In some embodiments, the agent is an antagonist of PD-L1. In some embodiments, the agent is an antagonist of PD-1. In some embodiments, an agent specifically binds PD-L1 and inhibits PD-L1 signaling. In some embodiments, an agent specifically binds PD-L1 and inhibits PD-1 signaling. In some embodiments, an agent specifically binds PD-L1 and is an antagonist of PD-L1-mediated signaling. In some embodiments, an agent specifically binds PD-L1 and is an antagonist of PD-1-mediated signaling. In some embodiments, an agent specifically binds PD-L1 and inhibits PD-1 activation.

In some embodiments of each of the aforementioned aspects and embodiments, as well as other aspects and embodiments described herein, an agent described herein specifically binds human PD-L1 and induces, activates, promotes, increases, enhances, and/or prolongs an immune response. In some embodiments, the immune response is directed to a tumor or tumor cell. In some embodiments, the agent increases cell-mediated immunity. In some embodiments, the agent increases T-cell activity. In some embodiments, the agent increases cytolytic T-cell (CTL) activity. In some embodiments, the agent increases natural killer (NK) cell activity. In some embodiments, the agent increases IL-2 production and/or the number of IL-2-producing cells. In some embodiments, the agent increases IFN-gamma production and/or the number of IFN-gamma-producing cells. In some embodiments, the agent increases a Th1-type immune response. In some embodiments, the agent decreases IL-4 production and/or the number of IL-4-producing cells. In some embodiments, the agent decreases IL-10 and/or the number of IL-10-producing cells. In some embodiments, the agent decreases a Th2-type immune response. In some embodiments, the agent decreases the number of Treg cells. In some embodiments, the agent decreases Treg activity. In some embodiments, the agent inhibits and/or decreases the suppressive activity of Tregs. In some embodiments, the agent decreases the number of myeloid-derived suppressor cells (MDSCs). In some embodiments, the agent decreases MDSC activity. In some embodiments, the agent inhibits and/or decreases the suppressive activity of MDSCs.

In some embodiments of each of the aforementioned aspects and embodiments, as well as other aspects and embodiments described herein, an agent described herein specifically binds PD-L1 and inhibits tumor growth. In some embodiments, the agent reduces tumor growth. In some embodiments, the agent reduces tumor growth to an undetectable size.

In another aspect, the invention provides compositions comprising an agent described herein. Methods of using a composition comprising an agent described herein are also provided.

In another aspect, the invention provides pharmaceutical compositions comprising an agent described herein and a pharmaceutically acceptable carrier. Methods of treating cancer and/or inhibiting tumor growth in a subject (e.g., a human) comprising administering to the subject an effective amount of a composition comprising an agent described herein are also provided.

In certain embodiments of each of the aforementioned aspects, as well as other aspects and/or embodiments described elsewhere herein, the agent is isolated. In certain embodiments, the agent is substantially pure.

In another aspect, the invention provides polynucleotides comprising a polynucleotide that encodes an agent described herein. In some embodiments, the polynucleotide is isolated. In some embodiments, the invention provides vectors that comprise the polynucleotides, as well as cells that comprise the vectors and/or the polynucleotides. In some embodiments, the invention also provides cells comprising or producing an agent described herein. In some embodiments, the cell is a monoclonal cell line.

In another aspect, the invention provides methods of modulating the immune response of a subject. In some embodiments, the method of modulating the immune response comprises a method of inducing, activating, promoting, increasing, enhancing, or prolonging an immune response in a subject. In some embodiments, a method of inducing, activating, promoting, increasing, enhancing, or prolonging an immune response in a subject, comprises administering a therapeutically effective amount of an antibody, bispecific agent, heterodimeric molecule, or polypeptide described herein. In some embodiments, a method of inducing, activating, promoting, increasing, enhancing, or prolonging an immune response in a subject, comprises administering a therapeutically effective amount of an antibody that specifically binds human PD-L1 described herein. In some embodiments, a method of inducing an immune response in a subject comprises administering an agent described herein. In some embodiments, a method of activating an immune response in a subject comprises administering an agent described herein. In some embodiments, a method of promoting an immune response in a subject comprises administering an agent described herein. In some embodiments, a method of increasing an immune response in a subject comprises administering an agent described herein. In some embodiments, a method of enhancing an immune response in a subject comprises administering an agent described herein. In some embodiments, a method of prolonging an immune response in a subject comprises administering an agent described herein. In some embodiments, the immune response is to an antigenic stimulation. In some embodiments, the antigenic stimulation is a tumor or a tumor cell. In some embodiments, the immune response is against a tumor or cancer.

In some embodiments, the invention provides methods of increasing the activity of immune cells. In some embodiments, a method of increasing the activity of immune cells comprises contacting the cells with an effective amount of an agent described herein. In some embodiments, the immune cells are T-cells, NK cells, monocytes, macrophages, myeloid-derived cells, antigen-presenting cells (APCs), and/or B-cells. In some embodiments, a method of increasing the activity of NK cells in a subject comprises administering to the subject a therapeutically effective amount of an agent described herein. In some embodiments, a method of increasing the activity of T-cells in a subject comprises administering to the subject a therapeutically effective amount of an agent described herein. In some embodiments, a method of increasing the activation of T-cells and/or NK cells in a subject comprises administering to the subject a therapeutically effective amount of an agent described herein. In some embodiments, a method of increasing the T-cell response in a subject comprises administering to the subject a therapeutically effective amount of an agent described herein. In some embodiments, a method of increasing the activity of CTLs in a subject comprises administering to the subject a therapeutically effective amount of an agent described herein. In some embodiments, a method of inhibiting the activity of Tregs in a subject comprises administering to the subject a therapeutically effective amount of an agent described herein. In some embodiments, a method of inhibiting the suppressive activity of Tregs in a subject comprises administering to the subject a therapeutically effective amount of an agent described herein. In some embodiments, a method of inhibiting the activity of MDSCs in a subject comprises administering to the subject a therapeutically effective amount of an agent described herein. In some embodiments, a method of inhibiting the suppressive activity of MDSCs in a subject comprises administering to the subject a therapeutically effective amount of an agent described herein.

In some embodiments, the invention provides methods of inducing, activating, promoting, increasing, enhancing, or prolonging an immune response in a subject, comprising administering to the subject a therapeutically effective amount of an agent that binds human PD-L1. In some embodiments, a method of inducing, activating, promoting, increasing, enhancing, or prolonging an immune response in a subject, comprises administering to the subject a therapeutically effective amount of an agent that inhibits or reduces PD-L1 or PD-1 activity. In some embodiments, a method of inducing, activating, promoting, increasing, enhancing, or prolonging an immune response in a subject, comprises administering to the subject a therapeutically effective amount of an agent that inhibits or reduces PD-L1 or PD-1 signaling. In some embodiments, the immune response is against a tumor cell, a tumor, or cancer.

In another aspect, the invention provides methods of inhibiting growth of tumor cells or a tumor comprising contacting the tumor or tumor cell with an effective amount of an agent described herein. In some embodiments, a method of inhibiting growth of a tumor comprises contacting a tumor or tumor cell with an effective amount of an agent that binds human PD-L1.

In another aspect, the invention provides methods of inhibiting growth of a tumor in a subject comprising administering to the subject a therapeutically effective amount of an agent described herein. In some embodiments, a method of inhibiting growth of a tumor in a subject comprises administering to the subject a therapeutically effective amount of an agent that binds human PD-L1. In some embodiments, a method of inhibiting growth of a tumor in a subject comprises administering to the subject a therapeutically effective amount of an antibody that binds human PD-L1. In some embodiments, a method of inhibiting growth of a tumor in a subject comprises administering to the subject a therapeutically effective amount of a bispecific agent that binds human PD-L1. In some embodiments, the tumor is selected from the group consisting of colorectal tumor, colon tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

In another aspect, the invention provides methods of treating cancer in a subject comprising administering to the subject a therapeutically effective amount of an agent described herein. In some embodiments, a method of treating cancer in a subject comprises administering to the subject a therapeutically effective amount of an agent that binds PD-L1. In some embodiments, a method of treating cancer in a subject comprises administering to the subject a therapeutically effective amount of an antibody that binds human PD-L1. In some embodiments, a method of treating cancer in a subject comprises administering to the subject a therapeutically effective amount of a bispecific agent that binds human PD-L1. In some embodiments, the cancer is selected from the group consisting of colorectal cancer, colon cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, and head and neck cancer.

In another aspect, the invention provides methods of stimulating a protective response in a subject comprising administering to the subject a therapeutically effective amount of an agent described herein in combination with an antigen of interest. In some embodiments, the antigen of interest is a tumor antigen. In some embodiments, the antigen of interest is a cancer cell biomarker. In some embodiments, the antigen of interest is a cancer stem cell biomarker.

In some embodiments of each of the aforementioned aspects and embodiments, as well as other aspects and embodiments described herein, a method further comprises administering at least one additional therapeutic agent. In some embodiments, the additional therapeutic agent is a chemotherapeutic agent. In some embodiments, the additional therapeutic agent is an antibody. In some embodiments, the additional therapeutic agent is an inhibitor of the Notch pathway, the Wnt pathway, or the RSPO/LGR pathway.

In some embodiments, the additional therapeutic agent is an immunotherapeutic agent. As used herein, the phrase “immunotherapeutic agent” is used in the broadest sense and refers to a substance that directly or indirectly affects or modulates the immune system. In some embodiments, an immunotherapeutic agent is an agent that directly or indirectly stimulates the immune system by inducing activation or increasing activity of one or more of components of the immune system. In some embodiments, an immunotherapeutic agent is an agent that directly or indirectly stimulates the immune system by reducing activation or decreasing activity of one or more components of the immune system. As the PD-L1-binding agents are considered immunotherapeutic agents, this additional immunotherapeutic agent may be considered a “second” immunotherapeutic agent. In some embodiments, the second immunotherapeutic agent is selected from the group consisting of: GM-CSF, M-CSF, G-CSF, IL-2, IL-3, IL-12, IL-15, B7-1 (CD80), B7-2 (CD86), 4-1BB ligand, GITRL, OX-40 ligand, anti-CD3 antibody, anti-CTLA-4 antibody, anti-CD28 antibody, anti-PD-1 antibody, anti-TIGIT antibody, anti-4-1BB antibody, anti-GITR antibody, anti-OX-40 antibody, anti-LAG-3 antibody, and anti-TIM-3 antibody.

In some embodiments of each of the aforementioned aspects and embodiments, as well as other aspects and embodiments described herein, the subject is human. In some embodiments, the subject has had a tumor or a cancer, at least partially, removed.

In some embodiments of each of the aforementioned aspects and embodiments, as well as other aspects and embodiments described herein, the tumor or the cancer expresses PD-L1. In some embodiments, a method further comprises a step of determining the level of PD-L1 expression in the tumor or cancer. In some embodiments, determining the level of PD-L1 expression is done prior to treatment or contact with an agent described herein. In some embodiments, if the tumor or cancer has an elevated expression level of PD-L1, an agent described herein is administered to the subject. In some embodiments, if the tumor or cancer has an elevated expression level of PD-L1, the tumor or cancer is contacted with an agent described herein.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE FIGURES

FIG. 1. FACS analysis of anti-PD-L1 antibodies binding human PD-L1. HEK-293T cells were transiently transfected with a cDNA expression vector encoding human PD-L1 ECD-CD4TM-GFP (green fluorescent protein). Transfected cells were incubated with anti-hPD-L1 antibodies and analyzed by flow cytometry. Specific binding is indicated by the presence of a diagonal signal within a FACS plot.

FIGS. 2A-2B. FACS analysis of anti-PD-L1 antibodies binding mouse or cynomolgus PD-L1. FIG. 2A. HEK-293T cells were transiently transfected with a cDNA expression vector encoding mouse PD-L1-CD4TM-GFP. FIG. 2B. HEK-293T cells were transiently transfected with a cDNA expression vector encoding cyno PD-L1-CD4TM-GFP. Transfected cells were incubated with anti-hPD-L1 antibodies and analyzed by flow cytometry. Specific binding is indicated by the presence of a diagonal signal within a FACS plot.

FIG. 3. FACS analysis of binding of PD-1 to PD-L1 in the presence of anti-PD-L1 antibodies. HEK-293T cells were transiently transfected with a cDNA expression vector encoding human PD-L1 ECD-CD4TM-GFP (green fluorescent protein). Transfected cells were incubated with soluble human PD-1-Fc fusion protein in the presence of antibodies generated to PD-L1 (332M1, 332M7, or 332M8) or no antibody and analyzed by flow cytometry. Specific binding is indicated by the presence of a diagonal signal within a FACS plot. Blocking of binding is demonstrated by the loss of specific binding within the FACS plot.

FIG. 4. Inhibition of tumor growth by anti-PD-L1 antibody. The human melanoma tumor OMP-M9 was implanted subcutaneously into humanized NSG mice (n=3 mice/group). Mice were injected twice a week with 10 mg/kg of anti-hPD-L1 antibody 332M1 (-●-) or a control antibody (-▾-). Tumor growth was monitored and tumor volumes were measured with electronic calipers at the indicated time points. Data is shown as tumor volume (mm³) over days post treatment. The figure shows the mean values±SEM for each group.

FIG. 5. Gene expression of CD8 and IFN-γ in tumor sample after treatment with anti-PD-L1 antibody.

### DETAILED DESCRIPTION OF THE INVENTION

The present invention provides novel agents, including, but not limited to, polypeptides, antibodies, bispecific antibodies, homodimeric bispecific molecules, and heterodimeric bispecific molecules that modulate the immune response. The agents include polypeptides, antibodies, bispecific antibodies, homodimeric bispecific molecules, and heterodimeric bispecific molecules that specifically bind PD-L1 and modulate PD-L1 or PD-1 activation and/or signaling. The agents include polypeptides, antibodies, bispecific antibodies, homodimeric bispecific molecules, and heterodimeric bispecific molecules that inhibit PD-L1 or PD-1 activation and/or signaling, thereby enhancing an immune response. Related polypeptides and polynucleotides, compositions comprising the agents, and methods of making the agents are also provided. Methods of screening for agents that modulate the immune response are provided. Methods of using the novel agents, such as methods of inhibiting tumor growth and/or methods of treating cancer are provided. Methods of using the novel agents, such as methods of activating an immune response, methods of stimulating an immune response, methods of promoting an immune response, methods of increasing an immune response, methods of activating natural killer (NK) cells and/or T-cells, methods of increasing the activity of NK cells and/or T-cells, methods of promoting the activity of NK cells and/or T-cells, methods of decreasing and/or inhibiting suppressor T-cells, and/or methods of decreasing and/or inhibiting myeloid-derived suppressor cells are further provided.

### I. Definitions

To facilitate an understanding of the present invention, a number of terms and phrases are defined below.

The terms “agonist” and “agonistic” as used herein refer to or describe an agent that is capable of, directly or indirectly, substantially inducing, activating, promoting, increasing, or enhancing the biological activity of a target and/or a pathway. The term “agonist” is used herein to include any agent that partially or fully induces, activates, promotes, increases, or enhances the activity of a protein.

The terms “antagonist” and “antagonistic” as used herein refer to or describe an agent that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein.

The terms “modulation” and “modulate” as used herein refer to a change or an alteration in a biological activity. Modulation includes, but is not limited to, stimulating or inhibiting an activity. Modulation may be an increase or a decrease in activity, a change in binding characteristics, or any other change in the biological, functional, or immunological properties associated with the activity of a protein, a pathway, a system, or other biological targets of interest.

The term “antibody” as used herein refers to an immunoglobulin molecule that recognizes and specifically binds a target through at least one antigen-binding site. The target may be a protein, polypeptide, peptide, carbohydrate, polynucleotide, lipid, or a combination of any of the foregoing. As used herein, the term encompasses intact polyclonal antibodies, intact monoclonal antibodies, antibody fragments (such as Fab, Fab′, F(ab′)2, and Fv fragments), single chain Fv (scFv) antibodies, multispecific antibodies, bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and generally refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site.

The term “variable region” of an antibody refers to the variable region of an antibody light chain or the variable region of an antibody heavy chain, either alone or in combination. Generally, the variable region of a heavy chain or a light chain consists of four framework regions connected by three complementarity determining regions (CDRs), also known as “hypervariable regions”. The CDRs in each chain are held together in close proximity by the framework regions and, with the CDRs from the other chain, contribute to the formation of the antigen-binding site(s) of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies that recognize different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody fragment, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to antibodies that are specific immunoglobulin chains, chimeric immunoglobulins, or fragments thereof that contain minimal non-human sequences. Typically, humanized antibodies are human immunoglobulins in which amino acid residues of the CDRs are replaced by amino acid residues from the CDRs of a non-human species (e.g., mouse, rat, rabbit, or hamster) that have the desired specificity, affinity, and/or binding capability. In some instances, the framework variable region amino acid residues of a human immunoglobulin may be replaced with the corresponding amino acid residues in an antibody from a non-human species. The humanized antibody can be further modified by the substitution of additional amino acid residues either in the framework variable region and/or within the replaced non-human amino acid residues to refine and optimize antibody specificity, affinity, and/or binding capability. The humanized antibody may comprise variable domains containing all or substantially all of the CDRs that correspond to the non-human immunoglobulin, whereas all or substantially all of the framework variable regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable regions of the light and heavy chains correspond to the variable regions of an antibody derived from one species of mammal (e.g., mouse, rat, rabbit, etc.) with the desired specificity, affinity, and/or binding capability, while the constant regions are homologous to the sequence in an antibody derived from another species. The constant regions are usually human to avoid eliciting an immune response in the antibody.

The terms “epitope” and “antigenic determinant” are used interchangeably herein and refer to that portion of an antigen or target capable of being recognized and specifically bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and often at least 5, 6, 7, or 8-10 amino acids in a unique spatial conformation.

The terms “selectively binds” or “specifically binds” mean that an agent interacts more frequently, more rapidly, with greater duration, with greater affinity, or with some combination of the above to the epitope, protein, or target molecule than with alternative substances, including related and unrelated proteins. In certain embodiments “specifically binds” means, for instance, that an agent binds a protein or target with a K_{D} of about 0.1 mM or less, but more usually less than about 1 μM. In certain embodiments, “specifically binds” means that an agent binds a target with a K_{D} of at least about 0.1 μM or less, at least about 0.01 μM or less, or at least about 1 nM or less. Because of the sequence identity between homologous proteins in different species, specific binding can include an agent that recognizes a protein or target in more than one species (e.g., mouse PD-L1 and human PD-L1). Likewise, because of homology within certain regions of polypeptide sequences of different proteins, specific binding can include an agent that recognizes more than one protein or target. It is understood that, in certain embodiments, an agent that specifically binds a first target may or may not specifically bind a second target. As such, “specific binding” does not necessarily require (although it can include) exclusive binding, i.e. binding to a single target. Thus, an agent may, in certain embodiments, specifically bind more than one target. In certain embodiments, multiple targets may be bound by the same antigen-binding site on the agent. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or other members of the immunoglobulin superfamily, in certain embodiments, a “polypeptide” can occur as a single chain or as two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, the coding region of a nucleotide sequence.

A “conservative amino acid substitution” is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies of the invention do not abrogate the binding of the polypeptide or antibody containing the amino acid sequence, to the target binding site. Methods of identifying nucleotide and amino acid conservative substitutions which do not eliminate binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “immune response” as used herein includes responses from both the innate immune system and the adaptive immune system. It includes cell-mediated and/or humoral immune responses. It includes, but is not limited to, both T-cell and B-cell responses, as well as responses from other cells of the immune system such as natural killer (NK) cells, myeloid-derived cells, monocytes, macrophages, etc.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at a new location. Generally, a “metastatic” or “metastasizing” cell is one that loses adhesive contacts with neighboring cells and migrates via the bloodstream or lymph from the primary site of disease to secondary sites throughout the body.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an appropriate host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into appropriate hosts (e.g., mice).

The term “subject” refers to any animal (e.g., a mammal), including, but not limited to, humans, non-human primates, canines, felines, rabbits, rodents, and the like, which is to be the recipient of a particular treatment. Typically, the terms “subject” and “patient” are used interchangeably herein in reference to a human subject.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a subject, together with at least one agent of the present disclosure, and which does not destroy the pharmacological activity thereof and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of an agent described herein, an antibody, a polypeptide, a polynucleotide, a small organic molecule, or other drug effective to “treat” a disease or disorder in a subject such as, a mammal. In the case of cancer or a tumor, the therapeutically effective amount of an agent (e.g., polypeptide or antibody) has a therapeutic effect and as such can enhance or boost the immune response, enhance or boost the anti-tumor response, increase cytolytic activity of immune cells, increase killing of tumor cells, increase killing of tumor cells by immune cells, reduce the number of tumor cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods of the present invention if the patient shows one or more of the following: an increased immune response, an increased anti-tumor response, increased cytolytic activity of immune cells, increased killing of tumor cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. PD-L1-Binding Agents

Programmed cell death-ligand 1 (PD-L1; also known as B7-H1 and CD274) is a type I transmembrane glycoprotein that contains an immunoglobulin-like variable (IgV) domain and an immunoglobulin-like constant (IgC2) domain. PD-L1 binds to programmed cell death-1 receptor (PD-1; CD279) with high affinity and to CD80 (B7-1). PD-1 is expressed on activated effector T-cells as well as natural killer cells and B-cells. High-level PD-1 expression is found on tumor-infiltrating lymphocytes (TILs) and on T-cells in chronic viral infections. PD-1 expression on effector T-cells is associated with constitutive antigen exposure and is considered to be a marker of T-cell unresponsiveness or exhaustion. PD-L1 is expressed by multiple cells, including T-cells, B-cells, antigen-presenting cells, dendritic cells, and macrophages. PD-L1 is also expressed by many different solid tumor types. The full-length amino acid (aa) sequence of human PD-L1 (UniProtKB No. Q9NZQ7) is known in the art and is provided herein as SEQ ID NO:1. As used herein, reference to amino acid positions refer to the numbering of the full-length amino acid sequence including the signal sequence.

The present invention provides agents that specifically bind PD-L1. These agents are referred to herein as “PD-L1-binding agents”. In some embodiments, the PD-L1-binding agent is a polypeptide. In some embodiments, the PD-L1-binding agent is an antibody. In some embodiments, the PD-L1-binding agent is a bispecific antibody. In some embodiments, the PD-L1-binding agent is a bispecific agent. In some embodiments, the PD-L1-binding agent is a homodimeric bispecific agent. In some embodiments, the PD-L1-binding agent is a heterodimeric bispecific agent. In some embodiments, the PD-L1-binding agent is a heterodimeric molecule. In some embodiments, the PD-L1-binding agent is a homodimeric molecule. In certain embodiments, the PD-L1-binding agent binds human PD-L1.

In some embodiments, an agent binds PD-L1 and interferes with the interaction of PD-L1 with a second protein. In some embodiments, an agent binds PD-L1 and interferes with the interaction of PD-L1 with PD-1. In some embodiments, an agent specifically binds PD-L1 and the agent disrupts binding of PD-L1 to PD-1, and/or disrupts PD-L1 activation of PD-1 signaling. In some embodiments, an agent binds PD-L1 and interferes with the interaction of PD-L1 with CD80. In some embodiments, an agent specifically binds PD-L1 and the agent disrupts binding of PD-L1 to CD80, and/or disrupts PD-L1 activation of CD80.

In certain embodiments, the PD-L1-binding agent is an antibody that specifically binds the extracellular domain of human PD-L1, or a fragment thereof. In some embodiments, the PD-L1-binding agent is an antibody that specifically binds an Ig-like domain of PD-L1. In some embodiments, the PD-L1-binding agent is an antibody that specifically binds the IgV domain of PD-L1. In some embodiments, the PD-L1-binding agent is an antibody that specifically binds the IgC2 domain of PD-L1. In some embodiments, the PD-L1-binding agent is an antibody that binds within amino acids 19-127 of human PD-L1. In some embodiments, the PD-L1-binding agent is an antibody that binds within amino acids 133-225 of human PD-L1. In some embodiments, the PD-L1-binding agent is an antibody that binds within amino acids 19-241 of human PD-L1. In some embodiments, the PD-L1-binding agent is an antibody that binds within amino acids 19-127 of SEQ ID NO:1. In some embodiments, the PD-L1-binding agent is an antibody that binds within amino acids 133-225 of SEQ ID NO:1. In some embodiments, the agent binds within amino acids 19-241 of SEQ ID NO:1. In certain embodiments, the PD-L1-binding agent binds within SEQ ID NO:3 or a fragment thereof.

In certain embodiments, the PD-L1-binding agent (e.g., an antibody) binds PD-L1 with a dissociation constant (K_{D}) of about 1 μM or less, about 100 nM or less, about 40 nM or less, about 20 nM or less, about 10 nM or less, about 1 nM or less, or about 0.1 nM or less. In certain embodiments, a PD-L1-binding agent binds PD-L1 with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a PD-L1-binding agent binds PD-L1 with a K_{D} of about 20 nM or less. In some embodiments, a PD-L1-binding agent binds PD-L1 with a K_{D} of about 10 nM or less. In some embodiments, a PD-L1-binding agent binds PD-L1 with a K_{D} of about 1 nM or less. In some embodiments, a PD-L1-binding agent binds PD-L1 with a K_{D} of about 0.5 nM or less. In some embodiments, a PD-L1-binding agent binds PD-L1 with a K_{D} of about 0.1 nM or less. In some embodiments, the dissociation constant of the binding agent (e.g., an antibody) to PD-L1 is the dissociation constant determined using a PD-L1 fusion protein comprising at least a portion of the extracellular domain of PD-L1 immobilized on a Biacore chip. In some embodiments, the dissociation constant of the binding agent (e.g., an antibody) to PD-L1 is the dissociation constant determined using the binding agent captured by an anti-human IgG antibody on a Biacore chip and a soluble PD-L1 protein.

In some embodiments, a PD-L1-binding agent comprises a first antigen-binding site that specifically binds PD-L1 and a second antigen-binding site that specifically binds a second target. In some embodiments, a PD-L1-binding agent is a bispecific agent that comprises a first antigen-binding site that specifically binds PD-L1 and a second antigen-binding site that specifically binds a second target. In some embodiments, a PD-L1-binding agent binds both PD-L1 and the second target with a K_{D} of about 100 nM or less. In some embodiments, a PD-L1-binding agent binds both PD-L1 and the second target with a K_{D} of about 50 nM or less. In some embodiments, a PD-L1-binding agent binds both PD-L1 and the second target with a K_{D} of about 20 nM or less. In some embodiments, a PD-L1-binding agent binds both PD-L1 and the second target with a K_{D} of about 10 nM or less. In some embodiments, a PD-L1-binding agent binds both PD-L1 and the second target with a K_{D} of about 1 nM or less. In some embodiments, the affinity of one of the antigen-binding sites may be weaker than the affinity of the other antigen-binding site. For example, the K_{D} of one antigen binding site may be about 1 nM and the K_{D} of the second antigen-binding site may be about 10 nM. In some embodiments, the difference in affinity between the two antigen-binding sites may be about 2-fold or more, about 3-fold or more, about 5-fold or more, about 8-fold or more, about 10-fold or more, about 15-fold or more, about 20-fold or more, about 30-fold or more, about 50-fold or more, or about 100-fold or more. Modulation of the affinities of the two antigen-binding sites may affect the biological activity of the bispecific antibody. For example, decreasing the affinity of the antigen-binding site for PD-L1 or the second target, may have a desirable effect, for example decreased toxicity of the binding agent and/or an increased therapeutic index.

In certain embodiments, the PD-L1-binding agent (e.g., an antibody) binds PD-L1 with a half maximal effective concentration (EC₅₀) of about 1 μM or less, about 100 nM or less, about 40 nM or less, about 20 nM or less, about 10 nM or less, about 1 nM or less, or about 0.1 nM or less. In certain embodiments, a PD-L1-binding agent binds to human PD-L1 with a half maximal effective concentration (EC₅₀) of about 1 μM or less, about 100 nM or less, about 40 nM or less, about 20 nM or less, about 10 nM or less, about 1 nM or less, or about 0.1 nM or less.

In certain embodiments, the PD-L1-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In certain embodiments, the antibody is an IgG1 antibody. In certain embodiments, the antibody is an IgG2 antibody. In some embodiments, the antibody is an IgG4 antibody. In certain embodiments, the antibody is an antibody fragment comprising an antigen-binding site. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody is conjugated to a cytotoxic moiety. In some embodiments, the antibody is isolated. In some embodiments, the antibody is substantially pure.

In some embodiments, the PD-L1-binding agents are polyclonal antibodies. Polyclonal antibodies can be prepared by any known method. In some embodiments, polyclonal antibodies are produced by immunizing an animal (e.g., a rabbit, rat, mouse, goat, donkey) with an antigen of interest (e.g., a purified peptide fragment, full-length recombinant protein, or fusion protein) using multiple subcutaneous or intraperitoneal injections. The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from the immunized animal, usually from blood or ascites. The polyclonal antibodies can be purified from serum or ascites according to standard methods in the art including, but not limited to, affinity chromatography, ion-exchange chromatography, gel electrophoresis, and dialysis.

In some embodiments, a PD-L1-binding agent is a monoclonal antibody. Monoclonal antibodies can be prepared using hybridoma methods known to one of skill in the art. In some embodiments, using the hybridoma method, a mouse, rat, rabbit, hamster, or other appropriate host animal, is immunized as described above to elicit the production of antibodies that specifically bind the immunizing antigen. In some embodiments, lymphocytes can be immunized in vitro. In some embodiments, the immunizing antigen can be a human protein or a fragment thereof. In some embodiments, the immunizing antigen can be a mouse protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen may be identified by a variety of methods including, but not limited to, immunoprecipitation, immunoblotting, and in vitro binding assays (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the nucleotides encoding the heavy and light chain variable regions of the antibody or the entire heavy and light chains, and their sequence is determined using standard techniques. The isolated polynucleotides encoding the heavy and light chain variable regions or heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In certain other embodiments, recombinant monoclonal antibodies, or fragments thereof, can be isolated from phage display libraries expressing variable domains or CDRs of a desired species.

The polynucleotide(s) encoding a monoclonal antibody can be modified, for example, by using recombinant DNA technology to generate alternative antibodies. In some embodiments, the constant domains of the light chain and heavy chain of, for example, a mouse monoclonal antibody can be substituted for constant regions of, for example, a human antibody to generate a chimeric antibody, or for a non-immunoglobulin polypeptide to generate a fusion antibody. In some embodiments, the constant regions are truncated or removed to generate a desired antibody fragment of a monoclonal antibody. Site-directed or high-density mutagenesis of the variable region(s) can be used to optimize specificity, affinity, etc. of a monoclonal antibody.

In some embodiments, a PD-L1-binding agent is a humanized antibody. Typically, humanized antibodies are human immunoglobulins in which the amino acid residues of the CDRs are replaced by amino acid residues from CDRs of a non-human species (e.g., mouse, rat, rabbit, hamster, etc.) that have the desired specificity, affinity, and/or binding capability using methods known to one skilled in the art. In some embodiments, some of the framework variable region amino acid residues of a human immunoglobulin are replaced with corresponding amino acid residues in an antibody from a non-human species. In some embodiments, a humanized antibody can be further modified by the substitution of additional residues either in the framework variable region and/or within the replaced non-human residues to refine and optimize antibody specificity, affinity, and/or capability. In general, a humanized antibody will comprise variable regions containing all, or substantially all, of the CDRs that correspond to the non-human immunoglobulin whereas all, or substantially all, of the framework regions are those of a human immunoglobulin sequence. In some embodiments, the framework regions are those of a human consensus immunoglobulin sequence. In some embodiments, a humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject.

In certain embodiments, a PD-L1-binding agent is a human antibody. Human antibodies can be directly prepared using various techniques known in the art. In some embodiments, human antibodies may be generated from immortalized human B lymphocytes immunized in vitro or from lymphocytes isolated from an immunized individual. In either case, cells that produce an antibody directed against a target antigen can be generated and isolated. In some embodiments, the human antibody can be selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies.

In some embodiments, human antibodies can be made in transgenic mice that contain human immunoglobulin loci. Upon immunization these mice are capable of producing the full repertoire of human antibodies in the absence of endogenous immunoglobulin production.

In some embodiments, the PD-L1-binding agent is a bispecific antibody. Thus, this invention encompasses bispecific antibodies that specifically recognize PD-L1 and at least one additional target. Bispecific antibodies are capable of specifically recognizing and binding at least two different antigens or epitopes. The different epitopes can either be within the same molecule (e.g., two epitopes on PD-L1) or on different molecules (e.g., one epitope on PD-L1 and one epitope on a different protein). In some embodiments, a bispecific antibody has enhanced potency as compared to an individual antibody or to a combination of more than one antibody. In some embodiments, a bispecific antibody has reduced toxicity as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g., a tumor and/or tumor microenvironment). In some embodiments, a bispecific antibody has the ability to concentrate the actions of two agents to a common target (e.g., a tumor or a tumor cell). In some embodiments, a bispecific antibody has the ability to target the actions of two agents to more than one biological pathway or function.

In some embodiments, the bispecific antibody is a monoclonal antibody. In some embodiments, the bispecific antibody is a humanized antibody. In some embodiments, the bispecific antibody is a human antibody. In some embodiments, the bispecific antibody is an IgG1 antibody. In some embodiments, the bispecific antibody is an IgG2 antibody. In some embodiments, the bispecific antibody is an IgG4 antibody. In some embodiments, the bispecific antibody has decreased toxicity and/or side effects. In some embodiments, the bispecific antibody has decreased toxicity and/or side effects as compared to a mixture of the two individual antibodies or the antibodies as single agents. In some embodiments, the bispecific antibody has an increased therapeutic index. In some embodiments, the bispecific antibody has an increased therapeutic index as compared to a mixture of the two individual antibodies or the antibodies as single agents.

In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA.

Techniques for making bispecific antibodies are known by those skilled in the art. In some embodiments, the bispecific antibodies comprise heavy chain constant regions with modifications in the amino acids which are part of the interface between the two heavy chains. In some embodiments, the bispecific antibodies can be generated using a “knobs-into-holes” strategy. In some cases, the “knobs” and “holes” terminology is replaced with the terms “protuberances” and “cavities”. In some embodiments, the bispecific antibodies may comprise variant hinge regions incapable of forming disulfide linkages between the heavy chains. In some embodiments, the modifications may comprise changes in amino acids that result in altered electrostatic interactions. In some embodiments, the modifications may comprise changes in amino acids that result in altered hydrophobic/hydrophilic interactions.

Bispecific antibodies can be intact antibodies or antibody fragments comprising antigen-binding sites. Antibodies with more than two valencies are also contemplated. For example, trispecific antibodies can be prepared. Thus, in certain embodiments the antibodies to PD-L1 are multispecific.

In certain embodiments, the antibodies (or other polypeptides) described herein may be monospecific. In certain embodiments, each of the one or more antigen-binding sites that an antibody contains is capable of binding (or binds) a homologous epitope on PD-L1.

In certain embodiments, a PD-L1-binding agent is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced by recombinant methods. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for PD-L1 or derivatives, fragments, analogs or homologs thereof. In some embodiments, antibody fragments are linear antibody fragments. In certain embodiments, antibody fragments are monospecific or bispecific. In certain embodiments, the PD-L1-binding agent is a scFv. Various techniques can be used for the production of single-chain antibodies specific to PD-L1.

In some embodiments, especially in the case of antibody fragments, an antibody is modified in order to alter (e.g., increase or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., PD-L1). In this regard, the variable region may comprise or be derived from any type of mammal that can be induced to mount a humoral response and generate immunoglobulins against the desired antigen. As such, the variable region of the modified antibodies can be, for example, of human, murine, rat, rabbit, non-human primate (e.g. cynomolgus monkeys, macaques, etc.), or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs may be derived from an antibody of different class and often from an antibody from a different species. It may not be necessary to replace all of the CDRs with all of the CDRs from the donor variable region to transfer the antigen binding capacity of one variable domain to another. Rather, it may only be necessary to transfer those residues that are required to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics. In some embodiments, the biochemical characteristic is increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain-deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding. Similarly, it may be desirable to simply delete the part of one or more constant region domains that control a specific effector function (e.g. complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or providing for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the modified antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase the serum half-life of the antibody. In other embodiments, the constant region modifications reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques.

In certain embodiments, a PD-L1-binding agent is an antibody that does not have one or more effector functions. For instance, in some embodiments, the antibody has no ADCC activity, and/or no complement-dependent cytotoxicity (CDC) activity. In certain embodiments, the antibody does not bind an Fc receptor, and/or complement factors. In certain embodiments, the antibody has no effector function(s).

The present invention further embraces variants and equivalents which are substantially homologous to the recombinant, monoclonal, chimeric, humanized, and human antibodies, or antibody fragments thereof, described herein. These variants can contain, for example, conservative substitution mutations, i.e. the substitution of one or more amino acids by similar amino acids.

The present invention provides methods for producing an antibody that binds PD-L1, including bispecific antibodies that specifically bind both PD-L1 and a second target. In some embodiments, the method for producing an antibody that binds PD-L1 comprises using hybridoma techniques. In some embodiments, a method for producing an antibody that binds human PD-L1 is provided. In some embodiments, the method comprises using a polypeptide comprising the extracellular domain of human PD-L1 or a fragment thereof as an antigen. In some embodiments, the method comprises using a polypeptide comprising amino acids 19-241 of human PD-L1 as an antigen. In some embodiments, the method comprises using a polypeptide comprising amino acids 19-241 of SEQ ID NO:1 as an antigen. In some embodiments, the method comprises using a polypeptide comprising amino acids 19-127 of human PD-L1 as an antigen. In some embodiments, the method comprises using a polypeptide comprising amino acids 19-127 of SEQ ID NO:1 as an antigen. In some embodiments, the method comprises using a polypeptide comprising amino acids 133-225 of human PD-L1 as an antigen. In some embodiments, the method comprises using a polypeptide comprising amino acids 133-225 of SEQ ID NO:1 as an antigen. In some embodiments, the method comprises using a polypeptide comprising SEQ ID NO:3 or a fragment thereof as an antigen. In some embodiments, the method of generating an antibody that binds PD-L1 comprises screening a human phage library. The present invention further provides methods of identifying an antibody that binds PD-L1. In some embodiments, the antibody is identified by FACS screening for binding to PD-L1 or a fragment thereof. In some embodiments, the antibody is identified by screening using ELISA for binding to PD-L1, or a fragment thereof. In some embodiments, the antibody is identified by screening by FACS for blocking of binding of PD-L1 to PD-1.

In some embodiments, a method of generating an antibody to PD-L1 comprises immunizing a mammal with a polypeptide comprising amino acids 19-241 of human PD-L1. In some embodiments, a method of generating an antibody to PD-L1 comprises immunizing a mammal with a polypeptide comprising a fragment of amino acids 19-241 of human PD-L1. In some embodiments, the method further comprises isolating antibodies or antibody-producing cells from the mammal. In some embodiments, a method of generating a monoclonal antibody which binds PD-L1 comprises: (a) immunizing a mammal with a polypeptide comprising a fragment of amino acids 19-241 of human PD-L1; (b) isolating antibody-producing cells from the immunized mammal; (c) fusing the antibody-producing cells with cells of a myeloma cell line to form hybridoma cells. In some embodiments, the method further comprises (d) selecting a hybridoma cell expressing an antibody that binds PD-L1. In certain embodiments, the mammal is a mouse. In some embodiments, the mammal is a rat. In some embodiments, the mammal is a rabbit. In some embodiments, the antibody is selected using a polypeptide comprising amino acids 19-241 or a fragment thereof of human PD-L1. In some embodiments, the antibody does not bind mouse PD-L1.

In some embodiments, a method of producing an antibody that binds PD-L1 comprises identifying an antibody using a membrane-bound heterodimeric molecule comprising a single antigen-binding site. In some non-limiting embodiments, the antibody is identified using methods and polypeptides described in International Publication WO 2011/100566.

In some embodiments, a method of producing an antibody that binds PD-L1 comprises screening an antibody-expressing library. In some embodiments, the antibody-expressing library is a phage library. In some embodiments, the screening comprises panning. In some embodiments, the antibody-expressing library is a mammalian cell library. In some embodiments, the antibody-expressing library is screened using amino acids 19-241 of human PD-L1 or a fragment thereof. In some embodiments, the antibody-expressing library is screened using amino acids 19-127 of human PD-L1. In some embodiments, the antibody-expressing library is screened using amino acids 133-225 of human PD-L1.

In some embodiments, the antibody generated by the methods described herein is a PD-L1 antagonist. In some embodiments, the antibody generated by the methods described herein blocks PD-L1 binding to PD-1. In some embodiments, the antibody generated by the methods described herein blocks PD-L1 binding to CD80. In some embodiments, the antibody generated by the methods described herein inhibits PD-L1 signaling. In some embodiments, the antibody generated by the methods described herein inhibits PD-1 signaling.

In certain embodiments, the antibodies described herein are isolated. In certain embodiments, the antibodies described herein are substantially pure.

The PD-L1-binding agents of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well-known in the art (see, e.g., Ausubel et al., Editors, 1994-present, Current Protocols in Molecular Biology, John Wiley & Sons, Inc., New York, N.Y.).

In a non-limiting example, screening for specific binding of an antibody to human PD-L1 may be determined using ELISA. An ELISA comprises preparing antigen (e.g., PD-L1 or a fragment thereof), coating wells of a 96-well microtiter plate with antigen, adding the test antibodies conjugated to a detectable compound such as an enzymatic substrate (e.g. horseradish peroxidase or alkaline phosphatase) to the well, incubating for a period of time and detecting the presence of an antibody bound to the antigen. In some embodiments, the test antibodies are not conjugated to a detectable compound, but instead a secondary antibody that recognizes the antibody (e.g., an anti-Fc antibody) and is conjugated to a detectable compound is added to the wells. In some embodiments, instead of coating the well with the antigen, the test antibodies can be coated to the wells, the antigen (e.g., PD-L1) is added to the wells, followed by a secondary antibody conjugated to a detectable compound. One of skill in the art would be knowledgeable as to the parameters that can be modified to increase the signal detected as well as other variations of ELISAs known in the art.

In another non-limiting example, the specific binding of an antibody to PD-L1 may be determined using FACS. A FACS screening assay may comprise generating a cDNA construct that expresses an antigen as a full-length protein (PD-L1) or a fusion protein (e.g., PD-L1-CD4TM), transfecting the construct into cells, expressing the antigen on the surface of the cells, mixing the test antibodies with the transfected cells, and incubating for a period of time. The cells bound by the test antibodies may be identified using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an antibody or other binding agent to an antigen (e.g., PD-L1) and the off-rate of an antibody-antigen interaction can be determined by competitive binding assays. One example of a competitive binding assay is a radioimmunoassay comprising the incubation of labeled antigen (e.g., ³H or ¹²⁵I-PD-L1), or fragment or variant thereof, with the antibody of interest in the presence of increasing amounts of unlabeled antigen followed by the detection of the antibody bound to the labeled antigen. The affinity of the antibody for the antigen and the binding off-rates can be determined from the data by Scatchard plot analysis. In some embodiments, Biacore kinetic analysis is used to determine the binding on and off rates of antibodies or agents that bind an antigen (e.g., PD-L1). In some embodiments, Biacore kinetic analysis comprises analyzing the binding and dissociation of antibodies from chips with immobilized antigen (e.g., PD-L1) on their surface. In some embodiments, Biacore kinetic analysis comprises analyzing the binding and dissociation of antigen (e.g., PD-L1) from chips with immobilized antibody (e.g., anti-PD-L1 antibody) on their surface.

In some embodiments, an antibody described herein specifically binds human PD-L1 and does not bind mouse PD-L1. In some embodiments, an antibody described herein specifically binds human PD-L1 and binds mouse PD-L1 at a level that is greatly reduced as compared to the binding of the antibody to human PD-L1. In some embodiments, an antibody described herein specifically binds human PD-L1 and specifically binds cynomolgus monkey PD-L1.

In certain embodiments, the invention provides a PD-L1-binding agent (e.g., an antibody) that specifically binds PD-L1, wherein the PD-L1-binding agent comprises one, two, three, four, five, and/or six of the CDRs of antibody 332M1 or 332M7 (see Table 1). In some embodiments, the PD-L1-binding agent comprises one or more of the CDRs of 332M1 or 332M7; two or more of the CDRs of 332M1 or 332M7; three or more of the CDRs of 332M1 or 332M7; four or more of the CDRs of 332M1 or 332M7; five or more of the CDRs of 332M1 or 332M7; or all six of the CDRs of 332M1 or 332M7.

TABLE1

| | 332M1 or 332M7 |
|---|---|
| HC CDR1 | TSYWMH (SEQ ID NO: 4) |
| HC CDR2 | AIYPGNSDTSYNQKFKG (SEQ ID NO: 5) |
| HC CDR3 | WGYGFDGAMDY (SEQ ID NO: 6) |
| LC CDR1 | RASQDIGSSLN (SEQ ID NO: 7) |
| LC CDR2 | ATSSLDS (SEQ ID NO: 8) |
| LC CDR3 | LQYASSP (SEQ ID NO: 9) |

In certain embodiments, the invention provides a PD-L1-binding agent (e.g., an antibody) that specifically binds PD-L1, wherein the PD-L1-binding agent comprises a heavy chain CDR1 comprising TSYWMH (SEQ ID NO:4), a heavy chain CDR2 comprising AIYPGNSDTSYNQKFKG (SEQ ID NO:5), and a heavy chain CDR3 comprising WGYGFDGAMDY (SEQ ID NO:6). In some embodiments, the PD-L1-binding agent further comprises a light chain CDR1 comprising RASQDIGSSLN (SEQ ID NO:7), a light chain CDR2 comprising ATSSLDS (SEQ ID NO:8), and a light chain CDR3 comprising LQYASSP (SEQ ID NO:9). In some embodiments, the PD-L1-binding agent comprises a light chain CDR1 comprising RASQDIGSSLN (SEQ ID NO:7), a light chain CDR2 comprising ATSSLDS (SEQ ID NO:8), and a light chain CDR3 comprising LQYASSP (SEQ ID NO:9). In some embodiments, the PD-L1-binding agent comprises: (a) a heavy chain CDR1 comprising TSYWMH (SEQ ID NO:4), a heavy chain CDR2 comprising AIYPGNSDTSYNQKFKG (SEQ ID NO:5), and a heavy chain CDR3 comprising WGYGFDGAMDY (SEQ ID NO:6); and (b) a light chain CDR1 comprising RASQDIGSSLN (SEQ ID NO:7), a light chain CDR2 comprising ATSSLDS (SEQ ID NO:8), and a light chain CDR3 comprising LQYASSP (SEQ ID NO:9).

In certain embodiments, the invention provides a PD-L1-binding agent (e.g., an antibody) that specifically binds human PD-L1, wherein the PD-L1-binding agent comprises: (a) a heavy chain CDR1 comprising TSYWMH (SEQ ID NO:4) or a variant thereof comprising 1, 2, 3, or 4 amino acid substitutions; (b) a heavy chain CDR2 comprising AIYPGNSDTSYNQKFKG (SEQ ID NO:5) or a variant thereof comprising 1, 2, 3, or 4 amino acid substitutions; (c) a heavy chain CDR3 comprising WGYGFDGAMDY (SEQ ID NO:6) or a variant thereof comprising 1, 2, 3, or 4 amino acid substitutions; (d) a light chain CDR1 comprising RASQDIGSSLN (SEQ ID NO:7) or a variant thereof comprising 1, 2, 3, or 4 amino acid substitutions; (e) a light chain CDR2 comprising ATSSLDS (SEQ ID NO:8) or a variant thereof comprising 1, 2, 3, or 4 amino acid substitutions; and (f) a light chain CDR3 comprising LQYASSP (SEQ ID NO:9) or a variant thereof comprising 1, 2, 3, or 4 amino acid substitutions. In certain embodiments, the amino acid substitutions are conservative substitutions. In some embodiments, the substitutions are made as part of a humanization process. In some embodiments, the substitutions are made as part of a germline humanization process.

In certain embodiments, the invention provides a PD-L1-binding agent (e.g., an antibody) that specifically binds PD-L1, wherein the PD-L1-binding agent comprises a heavy chain variable region having at least about 80% sequence identity to SEQ ID NO:10 or SEQ ID NO:14 and/or a light chain variable region having at least 80% sequence identity to SEQ ID NO:11 or SEQ ID NO:15. In certain embodiments, the PD-L1-binding agent comprises a heavy chain variable region having at least about 85%, at least about 90%, at least about 95%, at least about 97%, or at least about 99% sequence identity to SEQ ID NO:10. In certain embodiments, the PD-L1-binding agent comprises a heavy chain variable region having at least about 85%, at least about 90%, at least about 95%, at least about 97%, or at least about 99% sequence identity to SEQ ID NO:14. In certain embodiments, the PD-L1-binding agent comprises a light chain variable region having at least about 85%, at least about 90%, at least about 95%, at least about 97%, or at least about 99% sequence identity to SEQ ID NO:11. In certain embodiments, the PD-L1-binding agent comprises a light chain variable region having at least about 85%, at least about 90%, at least about 95%, at least about 97%, or at least about 99% sequence identity to SEQ ID NO:15. In certain embodiments, the PD-L1-binding agent comprises a heavy chain variable region having at least about 95% sequence identity to SEQ ID NO:10 or SEQ ID NO:14 and/or a light chain variable region having at least about 95% sequence identity to SEQ ID NO:11 or SEQ ID NO:15. In certain embodiments, the PD-L1-binding agent comprises a heavy chain variable region comprising SEQ ID NO:10 or SEQ ID NO:14 and/or a light chain variable region comprising SEQ ID NO:11 or SEQ ID NO:15. In certain embodiments, the PD-L1-binding agent comprises a heavy chain variable region comprising SEQ ID NO:10 or SEQ ID NO:14 and a light chain variable region comprising SEQ ID NO:11 or SEQ ID NO:15. In certain embodiments, the PD-L1-binding agent comprises a heavy chain variable region consisting essentially of SEQ ID NO:10 or SEQ ID NO:14 and a light chain variable region consisting essentially of SEQ ID NO:11 or SEQ ID NO:15. In certain embodiments, the PD-L1-binding agent comprises a heavy chain variable region consisting of SEQ ID NO:10 or SEQ ID NO:14 and a light chain variable region consisting of SEQ ID NO:11 or SEQ ID NO:15.

In certain embodiments, the PD-L1-binding agent comprises a heavy chain variable region comprising SEQ ID NO:10 and a light chain variable region comprising SEQ ID NO:11. In certain embodiments, the PD-L1-binding agent comprises of a heavy chain variable region consisting essentially of SEQ ID NO:10 and a light chain variable region consisting essentially of SEQ ID NO:11. In certain embodiments, the PD-L1-binding agent comprises a heavy chain variable region consisting of SEQ ID NO:10 and a light chain variable region consisting of SEQ ID NO:11.

In certain embodiments, the PD-L1-binding agent comprises a heavy chain variable region comprising SEQ ID NO:14 and a light chain variable region comprising SEQ ID NO:15. In certain embodiments, the PD-L1-binding agent comprises a heavy chain variable region consisting essentially of SEQ ID NO:14 and a light chain variable region consisting essentially of SEQ ID NO:15. In certain embodiments, the PD-L1-binding agent comprises a heavy chain variable region consisting of SEQ ID NO:14 and a light chain variable region consisting of SEQ ID NO:15.

In certain embodiments, the invention provides a PD-L1-binding agent (e.g., an antibody) that specifically binds PD-L1, wherein the PD-L1-binding agent comprises a heavy chain having at least 90% sequence identity to SEQ ID NO:17 or SEQ ID NO:19 and/or a light chain having at least 90% sequence identity to SEQ ID NO:21. In some embodiments, the PD-L1-binding agent comprises a heavy chain having at least 95% sequence identity to SEQ ID NO:17 or SEQ ID NO:19 and/or a light chain having at least 95% sequence identity to SEQ ID NO:21. In some embodiments, the PD-L1-binding agent comprises a heavy chain comprising SEQ ID NO:17 or SEQ ID NO:19 and/or a light chain comprising SEQ ID NO:21. In some embodiments, the PD-L1-binding agent comprises a heavy chain comprising SEQ ID NO:17 or SEQ ID NO:19 and a light chain comprising SEQ ID NO:21. In some embodiments, the PD-L1-binding agent comprises a heavy chain comprising SEQ ID NO:17 and a light chain comprising SEQ ID NO:21. In some embodiments, the PD-L1-binding agent comprises a heavy chain comprising SEQ ID NO:19 and a light chain comprising SEQ ID NO:21. In some embodiments, the PD-L1-binding agent comprises a heavy chain consisting essentially of SEQ ID NO:17 or SEQ ID NO:19 and a light chain consisting essentially of SEQ ID NO:21. In some embodiments, the PD-L1-binding agent comprises a heavy chain consisting essentially of SEQ ID NO:17 and a light chain consisting essentially of SEQ ID NO:21. In some embodiments, the PD-L1-binding agent comprises a heavy chain consisting essentially of SEQ ID NO:19 and a light chain consisting essentially of SEQ ID NO:21. In some embodiments, the PD-L1-binding agent comprises a heavy chain consisting of SEQ ID NO:17 or SEQ ID NO:19 and a light chain consisting of SEQ ID NO:21. In some embodiments, the PD-L1-binding agent comprises a heavy chain consisting of SEQ ID NO:17 and a light chain consisting of SEQ ID NO:21. In some embodiments, the PD-L1-binding agent comprises a heavy chain consisting of SEQ ID NO:19 and a light chain consisting of SEQ ID NO:21.

In certain embodiments, a PD-L1-binding agent comprises the heavy chain variable region and light chain variable region of the 332M1 antibody. In some embodiments, the PD-L1-binding agent comprises the variable regions of the 332M1 antibody wherein the heavy chain variable region and/or the light chain variable region from the 332M1 antibody have been affinity-matured. In certain embodiments, a PD-L1-binding agent comprises the heavy chain and light chain of the 332M1 antibody (with or without the leader sequence). In certain embodiments, a PD-L1-binding agent is the 332M1 antibody. In certain embodiments, a PD-L1-binding agent comprises the heavy chain variable region and/or the light chain variable region of the 332M1 antibody wherein the heavy chain variable region and/or the light chain variable region have been humanized. In certain embodiments, a PD-L1-binding agent comprises the heavy chain variable region and/or the light chain variable region of the 332M1 antibody in a humanized form. In certain embodiments, a PD-L1-binding agent comprises the heavy chain variable region of the 332M1 antibody as part of an IgG1, IgG2, or IgG4 heavy chain.

In certain embodiments, a PD-L1-binding agent comprises, consists essentially of, or consists of, the antibody 332M1. In certain embodiments, a PD-L1-binding agent comprises, consists essentially of, or consists of, a variant of the antibody 332M1.

In certain embodiments, a PD-L1-binding agent comprises the heavy chain variable region and light chain variable region of the 332M7 antibody. In some embodiments, the PD-L1-binding agent comprises the variable regions of the 332M7 antibody wherein the heavy chain variable region and/or the light chain variable region from the 332M7 antibody have been affinity-matured. In certain embodiments, a PD-L1-binding agent comprises the heavy chain and light chain of the 332M7 antibody (with or without the leader sequence). In certain embodiments, a PD-L1-binding agent is the 332M7 antibody. In certain embodiments, a PD-L1-binding agent comprises the heavy chain variable region of the 332M7 antibody as part of an IgG1, IgG2, or IgG4 heavy chain.

In certain embodiments, a PD-L1-binding agent comprises, consists essentially of, or consists of, the antibody 332M7. In certain embodiments, a PD-L1-binding agent comprises, consists essentially of, or consists of, a variant of the antibody 332M7.

In some embodiments, the PD-L1-binding agent comprises a heavy chain variable region encoded by the plasmid deposited with American Type Culture Collection (ATCC), 10801 University Boulevard, Manassas, Va., USA, under the conditions of the Budapest Treaty on Oct. 21, 2015, and designated PTA-122627. In some embodiments, the PD-L1-binding agent comprises a light chain variable region encoded by the plasmid deposited with ATCC, 10801 University Boulevard, Manassas, Va., USA, under the conditions of the Budapest Treaty on Oct. 21, 2015, and designated PTA-122628. In some embodiments, the PD-L1-binding agent comprises a heavy chain variable region encoded by the plasmid deposited with ATCC and designated PTA-122627 and a light chain variable region encoded by the plasmid deposited with ATCC and designated PTA-122628. In some embodiments, the PD-L1-binding agent comprises a light chain encoded by the plasmid deposited with ATCC and designated PTA-122628. In some embodiments, the PD-L1-binding agent comprises a heavy chain variable region encoded by the plasmid deposited with ATCC and designated PTA-122627 and a light chain encoded by the plasmid deposited with ATCC and designated PTA-122628.

This invention also encompasses homodimeric agents/molecules and heterodimeric agents/molecules. In some embodiments, the homodimeric agents are polypeptides. In some embodiments, the heterodimeric molecules are polypeptides. Generally, the homodimeric molecule comprises two identical polypeptides. Generally, the heterodimeric molecule comprises at least two different polypeptides. In some embodiments, the heterodimeric molecule is capable of binding at least two targets, e.g., a bispecific agent. The targets may be, for example, two different proteins on a single cell or two different proteins on two separate cells. The term “arm” may be used herein to describe the structure of a homodimeric molecule, a heterodimeric molecule, and/or a bispecific agent. In some embodiments, each arm comprises at least one polypeptide. Generally, each arm of a heterodimeric molecule has a different function, for example, binding two different targets. In some embodiments, one arm may comprise an antigen-binding site from an antibody. In some embodiments, one arm may comprise a binding portion of a receptor. In some embodiments, a homodimeric agent comprises two identical arms. In some embodiments, a heterodimeric agent comprises two different arms. In some embodiments, a bispecific agent comprises two different arms.

In some embodiments, a PD-L1-binding agent is a homodimeric molecule. In some embodiments, the homodimeric molecule comprises two identical polypeptides. In some embodiments, a PD-L1-binding agent is a heterodimeric molecule. In some embodiments, the heterodimeric molecule comprises at least two different polypeptides. In some embodiments, a PD-L1-binding agent is a heterodimeric agent. In some embodiments, a PD-L1-binding agent is a bispecific agent. In certain embodiments, the PD-L1-binding agent is a bispecific antibody.

In some embodiments, a homodimeric molecule is a homodimeric bispecific molecule. In some embodiments, a homodimeric bispecific molecule comprises a PD-L1-binding agent described herein. In some embodiments, the homodimeric bispecific agent comprises a fusion protein comprising (i) a polypeptide comprising an anti-PD-L1 antibody and (ii) a second polypeptide. In some embodiments, the homodimeric bispecific agent comprises a fusion protein comprising (i) a polypeptide comprising a single-chain anti-PD-L1 antibody and a second polypeptide. In some embodiments, the second polypeptide is a single chain TNFSF trimer. In some embodiments, the TNFSF is GITRL, CD40L, or OX40L. In some embodiments, the second polypeptide is a lymphokine.

In some embodiments, a heterodimeric molecule (e.g., a bispecific agent) comprises a PD-L1-binding agent described herein. In some embodiments, a heterodimeric molecule comprises at least two functions (i) binding to PD-L1 and (ii) binding to a second target. In some embodiments, a heterodimeric molecule comprises at least two functions (i) binding to PD-L1 and (ii) a “non-binding” function. In certain embodiments, a heterodimeric molecule comprises a second immunotherapeutic agent or functional fragment thereof. In some embodiments, one arm of the heterodimeric molecule comprises a PD-L1-binding agent described herein and one arm of the heterodimeric molecule comprises a second immunotherapeutic agent. In some embodiments, a second immunotherapeutic agent may include a cytokine, as well as various antigens including tumor antigens, and/or antigens derived from pathogens. In some embodiments, the immunotherapeutic agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), a cytokine (e.g., gamma-interferon), an antibody that blocks immunosuppressive functions (e.g., an anti-CTLA-4 antibody, anti-CD28 antibody, anti-PD-1 antibody, anti-PD-L1 antibody), an agonist antibody (e.g., an anti-GITR antibody, an anti-OX40 antibody), an agonist ligand (e.g., GITRL or OX40L), a toll-like receptor (e.g., TLR4, TLR7, TLR9), or a member of the B7 family (e.g., CD80, CD86).

In some embodiments, the PD-L1-binding agent is a heterodimeric molecule (e.g., a bispecific agent) that comprises a first CH3 domain and a second CH3 domain, each of which is modified to promote formation of heteromultimers. In some embodiments, the first and second CH3 domains are modified using a knobs-into-holes technique. In some embodiments, the first and second CH3 domains comprise changes in amino acids that result in altered electrostatic interactions. In some embodiments, the first and second CH3 domains comprise changes in amino acids that result in altered hydrophobic/hydrophilic interactions.

In some embodiments, the PD-L1-binding agent is a bispecific agent that comprises heavy chain constant regions selected from the group consisting of: (a) a first human IgG1 constant region, wherein the amino acids corresponding to positions 253 and 292 of IgG1 (SEQ ID NO:30) are replaced with glutamate or aspartate, and a second human IgG1 constant region, wherein the amino acids corresponding to positions 240 and 282 of IgG1 (SEQ ID NO:30) are replaced with lysine; (b) a first human IgG2 constant region, wherein the amino acids corresponding to positions 249 and 288 of IgG2 (SEQ ID NO:31) are replaced with glutamate or aspartate, and a second human IgG2 constant region wherein the amino acids corresponding to positions 236 and 278 of IgG2 (SEQ ID NO:31) are replaced with lysine; (c) a first human IgG3 constant region, wherein the amino acids corresponding to positions 300 and 339 of IgG3 (SEQ ID NO:32) are replaced with glutamate or aspartate, and a second human IgG3 constant region wherein the amino acids corresponding to positions 287 and 329 of IgG3 (SEQ ID NO:32) are replaced with lysine; and (d) a first human IgG4 constant region, wherein the amino acids corresponding to positions 250 and 289 of IgG4 (SEQ ID NO:33 or SEQ ID NO:34) are replaced with glutamate or aspartate, and a second IgG4 constant region wherein the amino acids corresponding to positions 237 and 279 of IgG4 (SEQ ID NO:33 or SEQ ID NO:34) are replaced with lysine.

In some embodiments, the PD-L1-binding agent is a bispecific agent which comprises a first human IgG1 constant region with amino acid substitutions at positions corresponding to positions 253 and 292 of IgG1 (SEQ ID NO:30), wherein the amino acids at positions corresponding to positions 253 and 292 of IgG1 (SEQ ID NO:30) are replaced with glutamate or aspartate, and a second human IgG1 constant region with amino acid substitutions at positions corresponding to positions 240 and 282 of IgG1 (SEQ ID NO:30), wherein the amino acids at positions corresponding to positions 240 and 282 of IgG1 (SEQ ID NO:30) are replaced with lysine. In some embodiments, the PD-L1-binding agent is a bispecific antibody which comprises a first human IgG2 constant region with amino acid substitutions at positions corresponding to positions 249 and 288 of IgG2 (SEQ ID NO:31), wherein the amino acids at positions corresponding to positions 249 and 288 of IgG2 (SEQ ID NO:31) are replaced with glutamate or aspartate, and a second human IgG2 constant region with amino acid substitutions at positions corresponding to positions 236 and 278 of IgG2 (SEQ ID NO:31), wherein the amino acids at positions corresponding to positions 236 and 278 of IgG2 (SEQ ID NO:31) are replaced with lysine. In some embodiments, the PD-L1-binding agent is a bispecific antibody which comprises a first human IgG3 constant region with amino acid substitutions at positions corresponding to positions 300 and 339 of IgG3 (SEQ ID NO:32), wherein the amino acids at positions corresponding to positions 300 and 339 of IgG3 (SEQ ID NO:32) are replaced with glutamate or aspartate, and a second human IgG3 constant region with amino acid substitutions at positions corresponding to positions 287 and 329 of IgG3 (SEQ ID NO:32), wherein the amino acids at positions corresponding to positions 287 and 329 of IgG3 (SEQ ID NO:32) are replaced with lysine. In some embodiments, the PD-L1-binding agent is a bispecific antibody which comprises a first human IgG4 constant region with amino acid substitutions at positions corresponding to positions 250 and 289 of IgG4 (SEQ ID NO:33 or SEQ ID NO:34), wherein the amino acids at positions corresponding to positions 250 and 289 of IgG4 (SEQ ID NO:33 or SEQ ID NO:34) are replaced with glutamate or aspartate, and a second human IgG4 constant region with amino acid substitutions at positions corresponding to positions 237 and 279 of IgG4 (SEQ ID NO:33 or SEQ ID NO:34), wherein the amino acids at positions corresponding to positions 237 and 279 of IgG4 (SEQ ID NO:33 or SEQ ID NO:34) are replaced with lysine.

In some embodiments, the PD-L1-binding agent is a bispecific agent which comprises a first human IgG1 constant region with amino acid substitutions at positions corresponding to positions 253 and 292 of IgG1 (SEQ ID NO:30), wherein the amino acids are replaced with glutamate, and a second human IgG1 constant region with amino acid substitutions at positions corresponding to positions 240 and 282 of IgG1 (SEQ ID NO:30), wherein the amino acids are replaced with lysine. In some embodiments, the PD-L1-binding agent is a bispecific antibody which comprises a first human IgG1 constant region with amino acid substitutions at positions corresponding to positions 253 and 292 of IgG1 (SEQ ID NO:30), wherein the amino acids are replaced with aspartate, and a second human IgG1 constant region with amino acid substitutions at positions corresponding to positions 240 and 282 of IgG1 (SEQ ID NO:30), wherein the amino acids are replaced with lysine.

In some embodiments, the PD-L1-binding agent is a bispecific agent which comprises a first human IgG2 constant region with amino acid substitutions at positions corresponding to positions 249 and 288 of IgG2 (SEQ ID NO:31), wherein the amino acids are replaced with glutamate, and a second human IgG2 constant region with amino acid substitutions at positions corresponding to positions 236 and 278 of IgG2 (SEQ ID NO:31), wherein the amino acids are replaced with lysine. In some embodiments, the PD-L1-binding agent is a bispecific antibody which comprises a first human IgG2 constant region with amino acid substitutions at positions corresponding to positions 249 and 288 of IgG2 (SEQ ID NO:31), wherein the amino acids are replaced with aspartate, and a second human IgG2 constant region with amino acid substitutions at positions corresponding to positions 236 and 278 of IgG2 (SEQ ID NO:31), wherein the amino acids are replaced with lysine.

In some embodiments, the PD-L1-binding agent is a bispecific agent which comprises a first human IgG4 constant region with amino acid substitutions at positions corresponding to positions 250 and 289 of IgG4 (SEQ ID NO:33 or SEQ ID NO:34), wherein the amino acids are replaced with glutamate, and a second human IgG4 constant region with amino acid substitutions at positions corresponding to positions 237 and 279 of IgG4 (SEQ ID NO:33 or SEQ ID NO:34), wherein the amino acids are replaced with lysine. In some embodiments, the PD-L1-binding agent is a bispecific antibody which comprises a first human IgG4 constant region with amino acid substitutions at positions corresponding to positions 250 and 289 of IgG4 (SEQ ID NO:33 or SEQ ID NO:34), wherein the amino acids are replaced with aspartate, and a second human IgG4 constant region with amino acid substitutions at positions corresponding to positions 237 and 279 of IgG4 (SEQ ID NO:33 or SEQ ID NO:34), wherein the amino acids are replaced with lysine.

In some embodiments, the PD-L1-binding agent is a bispecific antibody comprising a first antigen-binding site that specifically binds human PD-L1. In some embodiments, the PD-L1-binding agent is a bispecific antibody comprising a first antigen-binding site that specifically binds human PD-L1 and a second antigen-binding site that binds a second target. In some embodiments, the PD-L1-binding agent is a bispecific antibody comprising: a first antigen-binding site that specifically binds human PD-L1, wherein the first antigen-binding site comprises a heavy chain CDR1 comprising TSYWMH (SEQ ID NO:4), a heavy chain CDR2 comprising AIYPGNSDTSYNQKFKG (SEQ ID NO:5), and a heavy chain CDR3 comprising WGYGFDGAMDY (SEQ ID NO:6). In some embodiments, the PD-L1-binding agent is a bispecific antibody comprising: a first antigen-binding site that specifically binds human PD-L1, wherein the first antigen-binding site comprises (a) a heavy chain CDR1 comprising TSYWMH (SEQ ID NO:4), a heavy chain CDR2 comprising AIYPGNSDTSYNQKFKG (SEQ ID NO:5), and a heavy chain CDR3 comprising WGYGFDGAMDY (SEQ ID NO:6); and (b) a second antigen-binding site, wherein the first antigen-binding site and the second antigen-binding site comprise a common (i.e., identical) light chain. In some embodiments, the PD-L1-binding agent is a bispecific antibody comprising: a first antigen-binding site that specifically binds human PD-L1, wherein the first antigen-binding site comprises (a) a heavy chain CDR1 comprising TSYWMH (SEQ ID NO:4), a heavy chain CDR2 comprising AIYPGNSDTSYNQKFKG (SEQ ID NO:5), and a heavy chain CDR3 comprising WGYGFDGAMDY (SEQ ID NO:6), a light chain CDR1 comprising RASQDIGSSLN (SEQ ID NO:7), a light chain CDR2 comprising ATSSLDS (SEQ ID NO:8), and a light chain CDR3 comprising LQYASSP (SEQ ID NO:9); and (b) a second antigen-binding site. In some embodiments, the bispecific antibody comprises a first antigen-binding site comprising a light chain CDR1 comprising RASQDIGSSLN (SEQ ID NO:7), a light chain CDR2 comprising ATSSLDS (SEQ ID NO:8), and a light chain CDR3 comprising LQYASSP (SEQ ID NO:9).

In some embodiments, the PD-L1-binding agent is a bispecific antibody comprising a first heavy chain variable region having at least about 80% sequence identity to SEQ ID NO:10 or SEQ ID NO:14. In certain embodiments, the PD-L1-binding agent is a bispecific antibody comprising a first heavy chain variable region having at least about 85%, at least about 90%, at least about 95%, at least about 97%, or at least about 99% sequence identity to SEQ ID NO:10 or SEQ ID NO:14. In some embodiments, the bispecific antibody comprises a light chain variable region at least about 80% sequence identity to SEQ ID NO:11 or SEQ ID NO:15. In some embodiments, the bispecific antibody comprises a light chain variable region at least about 85%, at least about 90%, at least about 95%, at least about 97%, or at least about 99% sequence identity to SEQ ID NO:11 or SEQ ID NO:15. In some embodiments, the PD-L1-binding agent is a bispecific antibody comprising a first heavy chain variable region comprising SEQ ID NO:10. In some embodiments, the PD-L1-binding agent is a bispecific antibody comprising a first heavy chain variable region comprising SEQ ID NO:14. In some embodiments, the PD-L1-binding agent is a bispecific antibody comprising a first light chain variable region comprising SEQ ID NO:11. In some embodiments, the PD-L1-binding agent is a bispecific antibody comprising a first light chain variable region comprising SEQ ID NO:15.

In certain embodiments, the PD-L1-binding agent is a bispecific antibody that specifically binds human PD-L1 and a second target. In some embodiments, the second target is a tumor antigen. In some embodiments, the bispecific antibody comprises a PD-L1-binding antibody described herein and a second polypeptide comprising an antibody that specifically binds a tumor antigen. A bispecific antibody with a binding specificity for a tumor antigen can be used to direct the PD-L1-binding agent to a tumor. This may be useful to induce and/or enhance an immune response near or within the tumor microenvironment. In some embodiments, a bispecific antibody may be used to induce or enhance the activity of tumor infiltrating immune cells. In some embodiments, a bispecific antibody may be used to induce or enhance the activity of TILs. In some embodiments, a bispecific antibody may be used to inhibit or decrease the activity of Treg cells. In some embodiments, a bispecific antibody may be used to inhibit or decrease the activity of MSDCs.

In some embodiments, the PD-L1-binding agent is a bispecific antibody, wherein the first target is PD-L1 and the second target is on an immune response cell. In some embodiments, the second target is on a T-cell, a NK cell, a B-cell, an antigen-presenting cell, a macrophage, a dendritic cell, or a myeloid cell. In some embodiments, the second target is PD-1, TIGIT, CTLA-4, CD28, TIM-3, LAG-3, 4-1BB, GITR, CD40, or OX-40.

In some embodiments, a bispecific antibody comprises a first arm comprising a PD-L1-binding antibody described herein and a second arm comprising an antibody that specifically binds PD-1. In some embodiments, a bispecific antibody comprises a first arm comprising a PD-L1-binding antibody described herein and a second arm comprising an antibody that specifically binds TIGIT. In some embodiments, a bispecific antibody comprises a first arm comprising a PD-L1-binding antibody described herein and a second arm comprising an antibody that specifically binds GITR. In some embodiments, a bispecific antibody comprises a first arm comprising a PD-L1-binding antibody described herein and a second arm comprising an antibody that specifically binds OX-40. In some embodiments, a bispecific antibody comprises a first arm comprising a PD-L1-binding antibody described herein and a second arm comprising an antibody that specifically binds CTLA-4. In some embodiments, a bispecific antibody comprises a first arm comprising a PD-L1-binding antibody described herein and a second arm comprising an antibody that specifically binds CD28.

In some embodiments, the PD-L1-binding agent is a bispecific antibody that comprises a heavy chain variable region from the anti-PD-L1 antibody 332M7. In some embodiments, the PD-L1-binding agent is a bispecific antibody that comprises a light chain variable region from the anti-PD-L1 antibody 332M7.

In some embodiments, the PD-L1-binding agent is a bispecific agent, wherein the first target is PD-L1 and the second target is on an immune response cell. In some embodiments, the second target is on a T-cell, a NK cell, a B-cell, an antigen-presenting cell, a macrophage, a dendritic cell, or a myeloid cell. In some embodiments, the second target is PD-1, TIGIT, CTLA-4, CD28, TIM-3, LAG-3, 4-1BB, GITR, CD40, or OX-40.

In some embodiments, a bispecific agent comprises a first arm comprising a PD-L1-binding agent described herein and a second arm comprising a polypeptide comprising GITRL that specifically binds GITR. In some embodiments, a bispecific agent comprises a first arm comprising a PD-L1-binding agent described herein and a second arm comprising a polypeptide comprising OX-40L that specifically binds OX-40. In some embodiments, a bispecific agent comprises a first arm comprising a PD-L1-binding agent described herein and a second arm comprising a polypeptide comprising 4-1BB ligand that specifically binds 4-1BB.

In some embodiments, the PD-L1-binding agent is a bispecific agent that binds PD-L1 with a K_{D} of about 50 nM or less, about 25 nM or less, about 10 nM or less, about 1 nM or less, or about 0.1 nM or less. In some embodiments, the PD-L1-binding agent is a bispecific agent that binds a second target with a K_{D} of about 50 nM or less, about 25 nM or less, about 10 nM or less, about 1 nM or less, or about 0.1 nM or less. In some embodiments, the PD-L1-binding agent is a bispecific agent that binds PD-L1 with a K_{D} of about 50 nM or less and binds a second target with a K_{D} of about 50 nM or less. In some embodiments, the PD-L1-binding agent is a bispecific agent that binds PD-L1 with a K_{D} of about 25 nM or less and binds a second target with a K_{D} of about 25 nM or less. In some embodiments, the PD-L1 binding agent is a bispecific agent that binds PD-L1 with a K_{D} of about 10 nM or less and binds a second target with a K_{D} of about 10 nM or less. In some embodiments, the PD-L1-binding agent is a bispecific agent that binds PD-L1 with a K_{D} of about 1 nM or less and binds a second target with a K_{D} of about 1 nM or less.

In some embodiments, the PD-L1-binding agent is a bispecific agent which comprises one antigen-binding site with a binding affinity that is weaker than the binding affinity of the second antigen-binding site. For example, in some embodiments, the bispecific agent may bind PD-L1 with a K_{D} ranging from about 0.1 nM to 1 nM and may bind a second target with a K_{D} ranging from about 1 nM to 10 nM. Or the bispecific agent may bind PD-L1 with a K_{D} ranging from about 1 nM to 10 nM and may bind a second target with a K_{D} ranging from about 0.1 nM to 1 nM. In some embodiments, the bispecific agent may bind PD-L1 with a K_{D} ranging from about 0.1 nM to 1 nM and may bind a second target with a K_{D} ranging from about 1 nM to 10 nM. Or the bispecific agent may bind PD-L1 with a K_{D} ranging from about 1 nM to 10 nM and may bind a second target with a K_{D} ranging from about 0.1 nM to 1 nM. In some embodiments, the difference in affinity between the two antigen-binding sites may be about 2-fold or more, about 3-fold or more, about 5-fold or more, about 8-fold or more, about 10-fold or more, about 15-fold or more, about 30-fold or more, about 50-fold or more, or about 100-fold or more. In some embodiments, at least one amino acid residue in at least one CDR of the antigen-binding site for PD-L1 is substituted with a different amino acid so that the affinity of the PD-L1-binding site is altered. In some embodiments, the affinity of the PD-L1-binding site is increased. In some embodiments, the affinity of the PD-L1-binding site is decreased. In some embodiments, at least one amino acid residue in at least one CDR of the antigen-binding site for the second target is substituted with a different amino acid so that the affinity of the second antigen-binding site is altered. In some embodiments, the affinity of the second antigen-binding site is increased. In some embodiments, the affinity of the second antigen-binding site is decreased. In some embodiments, the affinities of both the PD-L1 and the second antigen-binding sites are altered.

The invention provides polypeptides, including, but not limited to, antibodies that specifically bind PD-L1. In some embodiments, a polypeptide binds human PD-L1.

In certain embodiments, a polypeptide comprises one, two, three, four, five, and/or six of the CDRs of antibody 332M1 or 332M7 (see Table 1 herein). In some embodiments, a polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the invention provides a polypeptide that specifically binds PD-L1, wherein the polypeptide comprises an amino acid sequence having at least about 80% sequence identity to SEQ ID NO:10 or SEQ ID NO:14, and/or an amino acid sequence having at least about 80% sequence identity to SEQ ID NO:11 or SEQ ID NO:15. In certain embodiments, the polypeptide comprises an amino acid sequence having at least about 85%, at least about 90%, at least about 95%, at least about 97%, or at least about 99% sequence identity to SEQ ID NO:10 or SEQ ID NO:14. In certain embodiments, the polypeptide comprises an amino acid sequence having at least about 85%, at least about 90%, at least about 95%, at least about 97%, or at least about 99% sequence identity to SEQ ID NO:11 or SEQ ID NO:14. In certain embodiments, the polypeptide comprises an amino acid sequence having at least about 95% sequence identity to SEQ ID NO:10 or SEQ ID NO:14 and/or an amino acid sequence having at least about 95% sequence identity to SEQ ID NO:11 or SEQ ID NO:15. In certain embodiments, the polypeptide comprises an amino acid sequence comprising SEQ ID NO:10 and/or an amino acid sequence comprising SEQ ID NO:11. In certain embodiments, the polypeptide comprises an amino acid sequence comprising SEQ ID NO:14 and/or an amino acid sequence comprising SEQ ID NO:15.

In some embodiments, a polypeptide comprises an amino acid sequence selected from the group consisting of: SEQ ID NO:10, SEQ ID NO:11, SEQ ID NO:14, SEQ ID NO:15, SEQ ID NO:16, SEQ ID NO:17, SEQ ID NO:18, SEQ ID NO:19, SEQ ID NO:20, and/or SEQ ID NO:21. As defined herein, a polypeptide can occur as a single chain or as two or more associated chains. In certain embodiments, a polypeptide comprises an amino acid sequence comprising SEQ ID NO:10 and an amino acid sequence comprising SEQ ID NO:11. In certain embodiments, a polypeptide comprises an amino acid sequence comprising SEQ ID NO:14 and an amino acid sequence comprising SEQ ID NO:15. In certain embodiments, a polypeptide comprises an amino acid sequence comprising SEQ ID NO:16 and an amino acid sequence comprising SEQ ID NO:20. In certain embodiments, the polypeptide comprises an amino acid sequence comprising SEQ ID NO:17 and an amino acid sequence comprising SEQ ID NO:21. In certain embodiments, a polypeptide comprises an amino acid sequence comprising SEQ ID NO:18 and an amino acid sequence comprising SEQ ID NO:20. In certain embodiments, the polypeptide comprises an amino acid sequence comprising SEQ ID NO:19 and an amino acid sequence comprising SEQ ID NO:21.

In certain embodiments, a polypeptide comprises an amino acid sequence consisting of SEQ ID NO:10 and an amino acid sequence consisting of SEQ ID NO:11. In certain embodiments, a polypeptide comprises an amino acid sequence consisting of SEQ ID NO:14 and an amino acid sequence consisting of SEQ ID NO:15. In certain embodiments, a polypeptide comprises an amino acid sequence consisting of SEQ ID NO:16 and an amino acid sequence consisting of SEQ ID NO:20. In certain embodiments, a polypeptide comprises an amino acid sequence consisting of SEQ ID NO:17 and an amino acid sequence consisting of SEQ ID NO:21. In certain embodiments, a polypeptide comprises an amino acid sequence consisting of SEQ ID NO:18 and an amino acid sequence consisting of SEQ ID NO:20. In certain embodiments, a polypeptide comprises an amino acid sequence consisting of SEQ ID NO:19 and an amino acid sequence consisting of SEQ ID NO:21.

Many proteins, including antibodies, contain a signal sequence that directs the transport of the proteins to various locations. Generally, signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell's outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or may be used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions as compared to a “native” or “parental” signal sequence. In some embodiments, the signal sequence of the polypeptide comprises amino acid substitutions and/or deletions that allow one cleavage site to be dominant, thereby resulting in a substantially homogeneous polypeptide with one N-terminus. In some embodiments, a signal sequence of the polypeptide affects the expression level of the polypeptide, e.g., increased expression or decreased expression.

In certain embodiments, an antibody competes for specific binding to PD-L1 with a PD-L1-binding agent described herein. In some embodiments, an antibody competes for specific binding to PD-L1 with a PD-L1-binding agent comprising: (a) a heavy chain CDR1 comprising TSYWMH (SEQ ID NO:4), a heavy chain CDR2 comprising AIYPGNSDTSYNQKFKG (SEQ ID NO:5), and a heavy chain CDR3 comprising WGYGFDGAMDY (SEQ ID NO:6) and (b) a light chain CDR1 comprising RASQDIGSSLN (SEQ ID NO:7), a light chain CDR2 comprising ATSSLDS (SEQ ID NO:8), and a light chain CDR3 comprising LQYASSP (SEQ ID NO:9).

In certain embodiments, an antibody competes for specific binding to PD-L1 with a PD-L1-binding agent comprising (a) a heavy chain variable region comprising SEQ ID NO:10 or SEQ ID NO:14 and (b) a light chain variable region comprising SEQ ID NO:11 or SEQ ID NO:14. In certain embodiments, an antibody competes for specific binding to PD-L1 with a PD-L1-binding agent comprising a heavy chain variable region comprising SEQ ID NO:10 and a light chain variable region comprising SEQ ID NO:11. In certain embodiments, an antibody competes for specific binding to PD-L1 with a PD-L1-binding agent comprising a heavy chain variable region comprising SEQ ID NO:14 and a light chain variable region comprising SEQ ID NO:15. In certain embodiments, an antibody competes for specific binding to PD-L1 with a PD-L1-binding agent comprising a heavy chain comprising SEQ ID NO:17 and a light chain comprising SEQ ID NO:21. In certain embodiments, an antibody competes for specific binding to PD-L1 with a PD-L1-binding agent comprising a heavy chain comprising SEQ ID NO:19 and a light chain comprising SEQ ID NO:21.

In certain embodiments, an antibody competes with antibody 332M7 for specific binding to PD-L1. In certain embodiments, an antibody competes with antibody 332M7 for specific binding to PD-L1. In some embodiments, an antibody competes with a reference antibody for specific binding to PD-L1, wherein the reference antibody is antibody 332M1. In some embodiments, an antibody competes with a reference antibody for specific binding to PD-L1, wherein the reference antibody is antibody 332M7.

In certain embodiments, an antibody binds the same epitope, or essentially the same epitope, on PD-L1 as a PD-L1-binding agent described herein. In certain embodiments, an antibody binds the same epitope, or essentially the same epitope, on PD-L1 as antibody 332M1. In certain embodiments, an antibody binds the same epitope, or essentially the same epitope, on PD-L1 as antibody 332M7.

In another embodiment, an antibody binds an epitope on PD-L1 that overlaps with the epitope on PD-L1 bound by a PD-L1-binding agent described herein. In some embodiments, the antibody binds an epitope on PD-L1 that overlaps with the epitope on PD-L1 bound by antibody 332M1. In another embodiment, the antibody binds an epitope on PD-L1 that overlaps with the epitope on PD-L1 bound by antibody 332M7.

In certain embodiments, the PD-L1-binding agent (e.g., an antibody) described herein binds PD-L1 and modulates PD-L1 activity. In some embodiments, the PD-L1-binding agent is a PD-L1 antagonist and decreases PD-L1 activity. In certain embodiments, the PD-L1-binding agent inhibits PD-L1 activity by at least about 10%, at least about 20%, at least about 30%, at least about 50%, at least about 75%, at least about 90%, or about 100%. In certain embodiments, a PD-L1-binding agent that inhibits PD-L1 activity is antibody 332M1 or antibody 332M7. In certain embodiments, a PD-L1-binding agent that inhibits human PD-L1 activity is a humanized version of antibody 332m1 (e.g., antibody 332M7).

In some embodiments, the PD-L1-binding agents described herein bind PD-L1 and inhibit or reduce PD-L1 signaling. In certain embodiments, the PD-L1-binding agent (e.g., an antibody) inhibits PD-L1 signaling by at least about 10%, at least about 20%, at least about 30%, at least about 50%, at least about 75%, at least about 90%, or about 100%. In some embodiments, the PD-L1-binding agent inhibits human PD-L1 signaling. In certain embodiments, a PD-L1-binding agent that inhibits PD-L1 signaling is antibody 332M1 or antibody 332M7.

In certain embodiments, the PD-L1-binding agent (e.g., antibody) inhibits binding of PD-L1 to a receptor. In certain embodiments, the PD-L1-binding agent inhibits binding of PD-L1 to PD-1. In some embodiments, the PD-L1-binding agent inhibits binding of PD-L1 to CD80. In certain embodiments, the inhibition of binding of a PD-L1-binding agent to PD-1 is at least about 10%, at least about 25%, at least about 50%, at least about 75%, at least about 90%, or at least about 95%. In certain embodiments, the inhibition of binding of a PD-L1-binding agent to CD80 is at least about 10%, at least about 25%, at least about 50%, at least about 75%, at least about 90%, or at least about 95%. In certain embodiments, a PD-L1-binding agent that inhibits binding of PD-L1 to PD-1 is antibody 332M1 or antibody 332M7. In certain embodiments, a PD-L1-binding agent that inhibits binding of PD-L1 to CD80 is antibody 332M1 or antibody 332M7.

In certain embodiments, the PD-L1-binding agent (e.g., antibody) blocks binding of PD-L1 to a receptor. In certain embodiments, the PD-L1-binding agent blocks binding of PD-L1 to PD-1. In certain embodiments, the blocking of binding of a PD-L1-binding agent to PD-1 is at least about 10%, at least about 25%, at least about 50%, at least about 75%, at least about 90%, or at least about 95%. In some embodiments, the PD-L1-binding agent blocks binding of PD-L1 to CD80. In certain embodiments, the blocking of binding of a PD-L1-binding agent to CD80 is at least about 10%, at least about 25%, at least about 50%, at least about 75%, at least about 90%, or at least about 95%. In certain embodiments, a PD-L1-binding agent that blocks binding of PD-L1 to PD-1 is antibody 332M1 or antibody 332M7. In certain embodiments, a PD-L1-binding agent that blocks binding of PD-L1 to CD80 is antibody 332M1 or antibody 332M7.

Binding assays are known to those of skill in the art and are described herein. Binding assays may be used to monitor the effect of a test agent on the interaction between a target protein and the protein's binding partner (e.g., receptor or ligand). For example, an in vitro binding assay can be used to evaluate if a PD-L1 antagonist blocks the interaction of PD-L1 to PD-1.

In certain embodiments, the PD-L1-binding agents described herein have one or more of the following effects: inhibit proliferation of tumor cells, inhibit tumor growth, reduce the tumorigenicity of a tumor, reduce the tumorigenicity of a tumor by reducing the frequency of cancer stem cells in the tumor, inhibit tumor growth, trigger cell death of tumor cells, enhance or boost the immune response, enhance or boost the anti-tumor response, increase cytolytic activity of immune cells, increase killing of tumor cells, increase killing of tumor cells by immune cells, induce cells in a tumor to differentiate, differentiate tumorigenic cells to a non-tumorigenic state, induce expression of differentiation markers in the tumor cells, prevent metastasis of tumor cells, decrease survival of tumor cells, increase cell contact-dependent growth inhibition, increase tumor cell apoptosis, reduce epithelial mesenchymal transition (EMT), or decrease survival of tumor cells.

In certain embodiments, the PD-L1-binding agents described herein inhibit tumor growth. In certain embodiments, the PD-L1-binding agents inhibit tumor growth in vivo (e.g., in a mouse model, and/or in a human having cancer). In certain embodiments, tumor growth is inhibited at least about two-fold, about three-fold, about five-fold, about ten-fold, about 50-fold, about 100-fold, or about 1000-fold as compared to a untreated tumor.

In certain embodiments, a PD-L1-binding agent described herein reduces the tumorigenicity of a tumor. In certain embodiments, the PD-L1-binding agent reduces the tumorigenicity of a tumor in an animal model, such as a mouse model. In some embodiments, the mouse model is a mouse xenograft model. In some embodiments, the mouse model is a humanized mouse model using a human tumor. In some embodiments, the mouse model is a humanized mouse model using a human patient-derived xenograft (PDX). In some embodiments, a PD-L1-binding agent does not bind mouse PD-L1 and is not effective in a mouse model. In some embodiments, a surrogate PD-L1-binding agent that binds mouse PD-L1 may be used in a mouse model. In certain embodiments, a PD-L1-binding agent reduces the tumorigenicity of a tumor comprising cancer stem cells in an animal model, such as a mouse model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236; U.S. Patent Publication No. 2008/0064049; and U.S. Patent Publication No. 2008/0178305.

In certain embodiments, the agents (e.g., polypeptides and/or antibodies) described herein bind PD-L1 and modulate an immune response. In some embodiments, a PD-L1-binding agent described herein activates and/or increases an immune response. In some embodiments, a PD-L1-binding agent increases, promotes, or enhances cell-mediated immunity. In some embodiments, a PD-L1-binding agent increases, promotes, or enhances innate cell-mediated immunity. In some embodiments, a PD-L1-binding agent increases, promotes, or enhances adaptive cell-mediated immunity. In some embodiments, a PD-L1-binding agent increases, promotes, or enhances T-cell activity. In some embodiments, a PD-L1-binding agent increases, promotes, or enhances cytolytic T-cell (CTL) activity. In some embodiments, a PD-L1-binding agent increases, promotes, or enhances NK cell activity. In some embodiments, a PD-L1-binding agent increases, promotes, or enhances lymphokine-activated killer cell (LAK) activity. In some embodiments, a PD-L1-binding agent increases, promotes, or enhances TIF activity. In some embodiments, a PD-L1-binding agent inhibits or decreases Treg cell activity. In some embodiments, a PD-L1-binding agent inhibits or decreases MDSC activity. In some embodiments, a PD-L1-binding agent increases, promotes, or enhances tumor cell killing. In some embodiments, a PD-L1-binding agent increases, promotes, or enhances the inhibition of tumor growth.

In certain embodiments, an agent described herein is an antagonist of human PD-L1. In some embodiments, the agent is an antagonist of PD-L1 and activates and/or increases an immune response. In some embodiments, the agent is an antagonist of PD-L1 and activates and/or increases activity of NK cells. In certain embodiments, the agent increases the activity by at least about 10%, at least about 20%, at least about 30%, at least about 50%, at least about 75%, at least about 90%, or about 100%. In some embodiments, the agent is an antagonist of PD-L1 and activates and/or increases activity of T-cells (e.g., T-cell cytolytic activity). In certain embodiments, the agent increases the activity by at least about 10%, at least about 20%, at least about 30%, at least about 50%, at least about 75%, at least about 90%, or about 100%. In some embodiments, the agent is an antagonist of PD-L1 and induces and/or enhances a Th1 immune response. In general, a Th1 immune response includes production of interferon-gamma (IFN-γ), IL-2, and tumor necrosis factor-beta (TNF-β). In comparison, a Th2 immune response generally includes production of IL-4, IL-5, IL-6, IL-9, IL-10, and IL-13. In some embodiments, the agent reduces and/or inhibits a Th2 response. In some embodiments, the agent is an antagonist of PD-L1 and induces and/or increases cytokine or lymphokine production. In some embodiments, the induction and/or increase in cytokine or lymphokines production may be an indirect effect.

In certain embodiments, a PD-L1-binding agent described herein increases activation of NK cells. In certain embodiments, a PD-L1-binding agent increases activation of T-cells. In certain embodiments, the activation of NK cells and/or T-cells by a PD-L1-binding agent results in an increase in the level of activation of NK cells and/or T-cells of at least about 10%, at least about 25%, at least about 50%, at least about 75%, at least about 90%, or at least about 95%.

In certain embodiments, the PD-L1-binding agent (e.g., antibody) is an antagonist of regulatory T-cell (Treg) activity. In certain embodiments, a PD-L1-binding agent described herein inhibits or decreases the activity of Tregs. In certain embodiments, the inhibition of activity of Tregs by a PD-L1-binding agent results in an inhibition of suppressive activity of a Treg cell of at least about 10%, at least about 25%, at least about 50%, at least about 75%, at least about 90%, at least about 95%, or about 100%. In certain embodiments, a PD-L1-binding agent that inhibits Treg activity is antibody 332M1 or antibody 332M7.

In certain embodiments, the PD-L1-binding agent (e.g., antibody) is an antagonist of MDSCs. In certain embodiments, the PD-L1-binding agent inhibits MDSC activity. In certain embodiments, the PD-L1-binding agent inhibits MDSC activity by at least about 10%, at least about 20%, at least about 30%, at least about 50%, at least about 75%, at least about 90%, or about 100%. In certain embodiments, a PD-L1-binding agent that inhibits MDSC activity is antibody 332M1 or antibody 332M7.

In certain embodiments, the PD-L1-binding agent (e.g., antibody) increases NK cell activity. In certain embodiments, the PD-L1-binding agent increases NK cell activity by at least about 10%, at least about 20%, at least about 30%, at least about 50%, at least about 75%, at least about 90%, or about 100%. In certain embodiments, a PD-L1-binding agent that increases NK cell activity is antibody 332M1 or antibody 332M7.

In certain embodiments, the PD-L1-binding agent (e.g., antibody) increases TIL activity. In certain embodiments, the PD-L1-binding agent increases TIL activity by at least about 10%, at least about 20%, at least about 30%, at least about 50%, at least about 75%, at least about 90%, or about 100%. In certain embodiments, a PD-L1-binding agent that increases TIL cell activity is antibody 332M1 or antibody 332M7.

In certain embodiments, the PD-L1-binding agent (e.g., antibody) increases or enhances LAK activity. In certain embodiments, the PD-L1-binding agent increases LAK activity by at least about 10%, at least about 20%, at least about 30%, at least about 50%, at least about 75%, at least about 90%, or about 100%. In certain embodiments, a PD-L1-binding agent that increases LAK cell activity is antibody 332M1 or antibody 332M7.

In vivo and in vitro assays for determining whether a PD-L1-binding agent (or candidate binding agent) modulates an immune response are known in the art or are being developed. In some embodiments, a functional assay that detects T-cell activation may be used. In some embodiments, a functional assay that detects T-cell proliferation may be used. In some embodiments, a functional assay that detects NK activity may be used. In some embodiments, a functional assay that detects CTL activity may be used. In some embodiments, a functional assay that detects Treg activity may be used. In some embodiments, a functional assay that detects MDSC activity may be used. In some embodiments, a functional assay that detects production of cytokines or lymphokines or cells producing cytokines or lymphokines may be used. In some embodiments, an ELISpot assay is used to measure antigen-specific T-cell frequency. In some embodiments, an ELISpot assay is used to measure cytokine release/production and/or used to measure the number of cytokine producing cells. In some embodiments, cytokine assays are used to identify a Th1 response. In some embodiments, cytokine assays are used to identify a Th2 response. In some embodiments, cytokine assays are used to identify a Th17 response. In some embodiments, FACS analysis is used to measure activation markers on immune cells, including but not limited to, T-cells, B-cells, NK cells, macrophages, and/or myeloid cells.

In certain embodiments, the PD-L1-binding agents described herein have a circulating half-life in mice, rats, cynomolgus monkeys, or humans of at least about 2 hours, at least about 5 hours, at least about 10 hours, at least about 24 hours, at least about 3 days, at least about 1 week, or at least about 2 weeks. In certain embodiments, the PD-L1-binding agent is an IgG (e.g., IgG1, IgG2, or IgG4) antibody that has a circulating half-life in mice, cynomolgus monkeys, or humans of at least about 2 hours, at least about 5 hours, at least about 10 hours, at least about 24 hours, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0. Known methods of increasing the circulating half-life of antibody fragments lacking the Fc region include such techniques as PEGylation.

In some embodiments described herein, the PD-L1-binding agents are polypeptides. In some embodiments, the polypeptides are recombinant polypeptides, natural polypeptides, or synthetic polypeptides comprising an antibody, or fragment thereof, that bind PD-L1. It will be recognized in the art that some amino acid sequences of the invention can be varied without significant effect of the structure or function of the protein. Thus, the invention further includes variations of the polypeptides which show substantial activity or which include regions of an antibody, or fragment thereof, that binds PD-L1. In some embodiments, amino acid sequence variations of PD-L1-binding polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve or otherwise modulate the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 22^{nd} Edition, 2012, Pharmaceutical Press, London.

In certain embodiments, the polypeptides described herein are isolated. In certain embodiments, the polypeptides described herein are substantially pure.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies, or fragments thereof, against human PD-L1. For example, recombinant expression vectors can be replicable DNA constructs which have synthetic or cDNA-derived DNA fragments encoding a polypeptide chain of a PD-L1-binding agent, such as an anti-PD-L1 antibody, or fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, in situations where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

The PD-L1-binding agents (e.g., polypeptides or antibodies) of the present invention can be expressed from one or more vectors. For example, in some embodiments, one heavy chain polypeptide is expressed by one vector, a second heavy chain polypeptide is expressed by a second vector and a light chain polypeptide is expressed by a third vector. In some embodiments, a first heavy chain polypeptide and a light chain polypeptide is expressed by one vector and a second heavy chain polypeptide is expressed by a second vector. In some embodiments, two heavy chain polypeptides are expressed by one vector and a light chain polypeptide is expressed by a second vector. In some embodiments, three polypeptides are expressed from one vector. Thus, in some embodiments, a first heavy chain polypeptide, a second heavy chain polypeptide, and a light chain polypeptide are expressed by a single vector.

Suitable host cells for expression of a PD-L1-binding polypeptide or antibody (or a PD-L1 protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts, as well as methods of protein production, including antibody production are well known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present invention provides cells comprising the PD-L1-binding agents described herein. In some embodiments, the cells produce the PD-L1-binding agents described herein. In certain embodiments, the cells produce an antibody. In some embodiments, the cells produce an antibody that binds human PD-L1. In certain embodiments, the cells produce antibody 332M126. In certain embodiments, the cells produce antibody 332M7. In some embodiments, the cells produce a bispecific antibody that binds PD-L1. In some embodiments, the cells produce a bispecific antibody that binds PD-L1 and a second target. In some embodiments, the cell is a hybridoma cell. In some embodiments, the cell is a mammalian cell. In some embodiments, the cell is a prokaryotic cell. In some embodiments, the cell is an eukaryotic cell.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Affinity chromatography used for purifying immunoglobulins can include Protein A, Protein G, and Protein L chromatography. Isolated proteins can be physically characterized using such techniques as proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF).

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a recombinant protein (e.g., a PD-L1-binding agent). Some or all of the foregoing purification steps, in various combinations, can be employed to provide a homogeneous recombinant protein.

In some embodiments, heterodimeric molecules such as bispecific antibodies are purified according the any of the methods described herein. In some embodiments, the heterodimeric molecules are isolated and/or purified using at least one chromatography step. In some embodiments, the at least one chromatography step comprises affinity chromatography. In some embodiments, the at least one chromatography step further comprises anion exchange chromatography. In some embodiments, the isolated and/or purified product comprises at least 90% of the heterodimeric molecule. In some embodiments, the isolated and/or purified product comprises at least 95%, 96%, 97%, 98% or 99% of the heterodimeric molecule. In some embodiments, the isolated and/or purified product comprises about 100% of the heterodimeric molecule.

In some embodiments, a polypeptide produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

In certain embodiments, the PD-L1-binding agent is a polypeptide that is not an antibody or does not comprise an immunoglobulin Fc region. A variety of methods for identifying and producing non-antibody polypeptides that bind with high affinity to a protein target are known in the art. In certain embodiments, phage or mammalian display technology may be used to produce and/or identify a PD-L1-binding polypeptide. In certain embodiments, the polypeptide comprises a protein scaffold of a type selected from the group consisting of protein A, protein G, a lipocalin, a fibronectin domain, an ankyrin consensus repeat domain, and thioredoxin. A variety of methods for identifying and producing non-antibody polypeptides that bind with high affinity to a protein target are known in the art. In certain embodiments, phage display technology may be used to produce and/or identify a binding polypeptide. In certain embodiments, mammalian cell display technology may be used to produce and/or identify a binding polypeptide.

Heteroconjugate molecules are also within the scope of the present invention. Heteroconjugate molecules are composed of two covalently joined polypeptides. Such molecules have, for example, been proposed to target immune cells to unwanted cells, such as tumor cells. It is also contemplated that the heteroconjugate molecules can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

In certain embodiments, the PD-L1-binding agents can be used in any one of a number of conjugated (i.e. an immunoconjugate or radioconjugate) or non-conjugated forms. In certain embodiments, the agents can be used in a non-conjugated form to harness the subject's natural defense mechanisms including CDC and ADCC to eliminate malignant or cancer cells.

In some embodiments, the PD-L1-binding agent is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. Conjugates of an antibody and one or more small molecule toxins, such as calicheamicins, maytansinoids, trichothenes, and CC1065, and the derivatives of these toxins that have toxin activity, can also be used. Conjugates of an antibody and cytotoxic agent may be made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. Polynucleotides

In certain embodiments, the invention encompasses polynucleotides comprising polynucleotides that encode an agent described herein. The term “polynucleotides that encode a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, the polynucleotide comprises a polynucleotide encoding a polypeptide comprising an amino acid sequence selected from the group consisting of: SEQ ID NO:10, SEQ ID NO:11, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

In certain embodiments, a polynucleotide comprises a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding an amino acid sequence selected from the group consisting of: SEQ ID NO:10, SEQ ID NO:11, SEQ ID NO:14, SEQ ID NO:15, SEQ ID NO:16, SEQ ID NO:17, SEQ ID NO:18, SEQ ID NO:19, SEQ ID NO:20, and SEQ ID NO:21. Also provided is a polynucleotide that comprises a polynucleotide that hybridizes to a polynucleotide encoding an amino acid sequence selected from the group consisting of: SEQ ID NO:10, SEQ ID NO:11, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

In some embodiments, the polynucleotide comprises a polynucleotide sequence selected from the group consisting of: SEQ ID NO:12, SEQ ID NO:13, SEQ ID NO:22, SEQ ID NO:23, SEQ ID NO:24, SEQ ID NO:25, SEQ ID NO:26, SEQ ID NO:27, SEQ ID NO:28, and SEQ ID NO:29. In certain embodiments, a polynucleotide comprises a polynucleotide having a nucleotide sequence at least about 80% identical, at least about 85% identical, at least about 90% identical, at least about 95% identical, and in some embodiments, at least about 96%, 97%, 98% or 99% identical to a nucleotide sequence selected from the group consisting of: SEQ ID NO:12, SEQ ID NO:13, SEQ ID NO:22, SEQ ID NO:23, SEQ ID NO:24, SEQ ID NO:25, SEQ ID NO:26, SEQ ID NO:27, SEQ ID NO:28, and SEQ ID NO:29. Also provided is a polynucleotide that comprises a polynucleotide that hybridizes to a polynucleotide sequence selected from the group consisting of: SEQ ID NO:12, SEQ ID NO:13, SEQ ID NO:22, SEQ ID NO:23, SEQ ID NO:24, SEQ ID NO:25, SEQ ID NO:26, SEQ ID NO:27, SEQ ID NO:28, and SEQ ID NO:29.

In certain embodiments, hybridization techniques are conducted under conditions of high stringency. Conditions of high stringency are known to those of skill in the art and may include but are not limited to, (1) employ low ionic strength and high temperature for washing, for example 15 mM sodium chloride/1.5 mM sodium citrate (1×SSC) with 0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes in 0.2×SSC containing 50% formamide at 55° C., followed by a high-stringency wash consisting of 0.1×SSC containing EDTA at 55° C.

In certain embodiments, a polynucleotide comprises the coding sequence of the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a pre-protein and can have the leader sequence cleaved by the host cell to form the mature form of the polypeptide. The polynucleotides can also encode for a pro-protein which is the mature protein plus additional 5′ amino acid residues. A mature protein having a pro-sequence is a pro-protein and is an inactive form of the protein. Once the pro-sequence is cleaved an active mature protein remains.

In certain embodiments, a polynucleotide comprises the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag which can be used in conjunction with other affinity tags.

The present invention further relates to variants of the polynucleotides described herein, where the variants encode, for example, fragments, analogs, and/or derivatives.

In certain embodiments, the present invention provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a PD-L1-binding agent described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, at least one polynucleotide variant is produced (without changing the amino acid sequence of the encoded polypeptide) to increase production of a heterodimeric molecule.

In some embodiments, at least one polynucleotide variant is produced (without changing the amino acid sequence of the encoded polypeptide) to increase production of a bispecific antibody.

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

### IV. Methods of Use and Pharmaceutical Compositions

The PD-L1-binding agents of the invention are useful in a variety of applications including, but not limited to, therapeutic treatment methods, such as the treatment of cancer. In some embodiments, the therapeutic treatment methods comprise immunotherapy. In certain embodiments, a PD-L1-binding agent is useful for activating, promoting, increasing, and/or enhancing an immune response, inhibiting tumor growth, reducing tumor volume, increasing tumor cell apoptosis, and/or reducing the tumorigenicity of a tumor. The methods of use may be in vitro, ex vivo, or in vivo methods.

The present invention provides methods for activating an immune response in a subject using a PD-L1-binding agent described herein. In some embodiments, the invention provides methods for promoting an immune response in a subject using a PD-L1-binding agent described herein. In some embodiments, the invention provides methods for increasing an immune response in a subject using a PD-L1-binding agent described herein. In some embodiments, the invention provides methods for enhancing an immune response in a subject using a PD-L1-binding agent described herein. In some embodiments, the activating, promoting, increasing, and/or enhancing of an immune response comprises increasing cell-mediated immunity. In some embodiments, the activating, promoting, increasing, and/or enhancing of an immune response comprises increasing T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CTL activity and increasing NK cell activity. In some embodiments, the activating, promoting, increasing, and/or enhancing of an immune response comprises inhibiting or decreasing the suppressive activity of Tregs. In some embodiments, the activating, promoting, increasing, and/or enhancing of an immune response comprises inhibiting or decreasing the suppressive activity of MDSCs. In some embodiments, the immune response is a result of antigenic stimulation. In some embodiments, the antigenic stimulation is a tumor cell. In some embodiments, the antigenic stimulation is cancer.

In some embodiments, a method of increasing an immune response in a subject comprises administering to the subject a therapeutically effective amount of a PD-L1-binding agent described herein, wherein the agent is an antibody that specifically binds the extracellular domain of PD-L1. In some embodiments, a method of increasing an immune response in a subject comprises administering to the subject a therapeutically effective amount of a PD-L1-binding agent described herein, wherein the agent is an antibody that specifically binds the extracellular domain of human PD-L1.

The invention also provides methods of inhibiting and/or reducing PD-L1 signaling in a cell comprising contacting the cell with an effective amount of a PD-L1-binding agent described herein. In certain embodiments, the cell is a T-cell. In some embodiments, the cell is an activated T-cell. In some embodiments, the cell is a NK cell. In some embodiments, the cell is a Treg. In some embodiments, the cell is a MDSC. In certain embodiments, the method is an in vivo method wherein the step of contacting the cell with the agent comprises administering a therapeutically effective amount of the PD-L1-binding agent to the subject. In some embodiments, the method is an in vitro or ex vivo method.

The present invention also provides methods for inhibiting growth of a tumor using a PD-L1-binding agent described herein. In certain embodiments, the method of inhibiting growth of a tumor comprises contacting a cell mixture with a PD-L1-binding agent in vitro. For example, an immortalized cell line or a cancer cell line mixed with immune cells (e.g., T-cells or NK cells) is cultured in medium to which is added a test agent that binds PD-L1. In some embodiments, tumor cells are isolated from a patient sample such as, for example, a tissue biopsy, pleural effusion, or blood sample, mixed with immune cells (e.g., T-cells and/or NK cells), and cultured in medium to which is added a test agent that binds PD-L1. In some embodiments, a PD-L1-binding agent increases, promotes, and/or enhances the activity of the immune cells. In some embodiments, a PD-L1-binding agent inhibits tumor cell growth.

In some embodiments, the method of inhibiting growth of a tumor comprises contacting the tumor or tumor cells with a PD-L1-binding agent described herein in vivo. In certain embodiments, contacting a tumor or tumor cell with a PD-L1-binding agent is undertaken in an animal model. For example, a test agent may be administered to mice which have tumors. In some embodiments, a PD-L1-binding agent increases, promotes, and/or enhances the activity of immune cells in the mice. In some embodiments, a PD-L1-binding agent inhibits tumor growth. In some embodiments, a PD-L1-binding agent causes a tumor to regress. In some embodiments, a PD-L1-binding agent is administered at the same time or shortly after introduction of tumor cells into the animal to prevent tumor growth (“preventative model”). In some embodiments, a PD-L1-binding agent is administered as a therapeutic after tumors have grown to a specified size or have become “established” (“therapeutic model”).

In certain embodiments, the method of inhibiting growth of a tumor comprises administering to a subject a therapeutically effective amount of a PD-L1-binding agent described herein. In certain embodiments, the subject is a human. In certain embodiments, the subject has a tumor or the subject had a tumor which was at least partially removed.

In addition, the invention provides a method of inhibiting growth of a tumor in a subject, comprising administering to the subject a therapeutically effective amount of a PD-L1-binding agent described herein. In certain embodiments, the tumor comprises cancer stem cells. In certain embodiments, the frequency of cancer stem cells in the tumor is reduced by administration of the agent. In some embodiments, a method of reducing the frequency of cancer stem cells in a tumor in a subject, comprising administering to the subject a therapeutically effective amount of a PD-L1-binding agent is provided.

In addition, the invention provides a method of reducing the tumorigenicity of a tumor in a subject, comprising administering to the subject a therapeutically effective amount of a PD-L1-binding agent described herein. In certain embodiments, the tumor comprises cancer stem cells. In some embodiments, the tumorigenicity of a tumor is reduced by reducing the frequency of cancer stem cells in the tumor. In some embodiments, the methods comprise using the PD-L1-binding agents described herein. In certain embodiments, the frequency of cancer stem cells in the tumor is reduced by administration of a PD-L1-binding agent described herein.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic tumor. In certain embodiments, the tumor is a melanoma tumor.

The present invention further provides methods for treating cancer in a subject comprising administering to the subject a therapeutically effective amount of a PD-L1-binding agent described herein. In some embodiments, a PD-L1-binding agent binds human PD-L1 and inhibits or reduces growth of the cancer.

The present invention provides for methods of treating cancer comprising administering to a subject a therapeutically effective amount of a PD-L1-binding agent described herein (e.g., a subject in need of treatment). In certain embodiments, the subject is a human. In certain embodiments, the subject has a cancerous tumor. In certain embodiments, the subject has had a tumor at least partially removed.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma.

In some embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

In some embodiments of the methods described herein, a method further comprises a step of determining the level of PD-L1 expression in the tumor or cancer. In some embodiments, the determining of the level of PD-L1 expression is done prior to treatment with a PD-L1-binding agent described herein. In some embodiments, if a tumor or cancer has an elevated expression level of PD-L1, a PD-L1-binding agent is administered to the subject. In some embodiments, a method comprises (i) obtaining a sample of a subject's cancer or tumor; (ii) measuring the expression level of PD-L1 in the sample; and (iii) administering an effective amount of a PD-L1-binding agent to the subject if the tumor or cancer has an elevated expression level of PD-L1. In some embodiments, the sample is a biopsy sample. In some embodiments, the sample comprises tumor cells, tumor infiltrating immune cells, stromal cells, and any combination thereof. In some embodiments, the sample is a formalin-fixed paraffin embedded (FFPE) sample. In some embodiments, the sample is archival, fresh, or frozen tissue. In some embodiments, the expression level of PD-L1 in the sample is compared to a pre-determined expression level of PD-L1. In some embodiments, the pre-determined expression level of PD-L1 expression is an expression level of PD-L1 in a reference tumor sample, a reference normal tissue sample, a series of reference tumor samples, or a series of reference normal tissue samples. In some embodiments, the expression level of PD-L1 is determined using an immunohistochemistry (IHC) assay. In some embodiments, the expression level of PD-L1 is determined using an assay which comprises an H-score evaluation. In some embodiments, the expression level of PD-L1 is determined using an antibody that specifically binds PD-L1. In some embodiments, PD-L1 is detected on tumor cells. In some embodiments, PD-L1 is detected on tumor infiltrating immune cells. In some embodiments, PD-L1 is detected on TILs.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a PD-L1-binding agent described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that may be administered in combination with the PD-L1-binding agents described herein include chemotherapeutic agents. Thus, in some embodiments, the method or treatment involves the administration of an agent of the present invention in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4^{th} Edition, 2008, M. C. Perry, Editor, Lippincott, Williams & Wilkins, Philadelphia, Pa.

Useful classes of chemotherapeutic agents that may be used in combination with a PD-L1-binding agent include, but are not limited to, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents that may be used in combination with a PD-L1-binding agent include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel.

In some embodiments, an additional therapeutic agent comprises an agent such as a small molecule. In some embodiments, treatment can involve the combined administration of a PD-L1-binding agent with a small molecule that acts as an inhibitor against tumor-associated antigens including, but not limited to, EGFR, HER2 (ErbB2), and/or VEGF. In some embodiments, a PD-L1-binding agent is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments, the additional therapeutic agent is an agent that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the RSPO/LGR pathway. In some embodiments, the additional therapeutic agent is an inhibitor of the mTOR/AKR pathway.

In some embodiments, an additional therapeutic agent comprises a biological molecule, such as an antibody. For example, treatment can involve the combined administration of a PD-L1-binding agent with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits I3-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In certain embodiments, an additional therapeutic agent comprises a second immunotherapeutic agent. In some embodiments, the additional immunotherapeutic agent includes, but is not limited to, a colony stimulating factor, an interleukin, an antibody that blocks immunosuppressive functions (e.g., an anti-CTLA-4 antibody, anti-CD28 antibody, anti-CD3 antibody, anti-PD-1 antibody), an antibody that enhances immune cell functions (e.g., an anti-GITR antibody, an anti-OX-40 antibody, or an anti-4-1BB antibody), a toll-like receptor (e.g., TLR4, TLR7, TLR9), a soluble ligand (e.g., GITRL, OX-40L, or 4-1BB ligand), or a member of the B7 family (e.g., CD80, CD86).

In some embodiments, the additional therapeutic agent is an antibody that is an immune checkpoint inhibitor. In some embodiments, the immune checkpoint inhibitor is an anti-PD-1 antibody, an anti-CTLA-4 antibody, an anti-CD28 antibody, an anti-LAG3 antibody, an anti-TIM3 antibody, an anti-GITR antibody, an anti-4-1BB antibody, or an anti-OX-40 antibody. In some embodiments, the additional therapeutic agent is an anti-PD-1 antibody selected from the group consisting of: nivolumab (OPDIVO), pembrolizumab (KEYTRUDA), pidilzumab, MEDI0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MEDI4736), and avelumab (MSB0010718C). In some embodiments, the additional therapeutic agent is an anti-CTLA-4 antibody selected from the group consisting of: ipilimumab (YERVOY) and tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566.

In some embodiments, a PD-L1-binding agent can be administered in combination with a biologic molecule selected from the group consisting of: adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF-β, TNF-α, VEGF, PlGF, gamma-IFN, IL-1, IL-2, IL-3, IL-4, IL-5, IL-6, IL-7, IL-12, IL-15, and IL-18.

In some embodiments, treatment with a PD-L1-binding agent described herein can be accompanied by surgical removal of tumors, removal of cancer cells, or any other surgical therapy deemed necessary by a treating physician.

In certain embodiments, treatment involves the administration of a PD-L1-binding agent described herein in combination with radiation therapy. Treatment with a PD-L1-binding agent can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously.

It will be appreciated that the combination of a PD-L1-binding agent described herein and at least one additional therapeutic agent may be administered in any order or concurrently. In some embodiments, a PD-L1-binding agent will be administered to patients that have previously undergone treatment with a second therapeutic agent. In certain other embodiments, a PD-L1-binding agent and a second therapeutic agent will be administered substantially simultaneously or concurrently. For example, a subject may be given an agent while undergoing a course of treatment with a second therapeutic agent (e.g., chemotherapy). In certain embodiments, a PD-L1-binding agent will be administered within 1 year of the treatment with a second therapeutic agent. In certain alternative embodiments, a PD-L1-binding agent will be administered within 10, 8, 6, 4, or 2 months of any treatment with a second therapeutic agent. In certain other embodiments, a PD-L1-binding agent will be administered within 4, 3, 2, or 1 weeks of any treatment with a second therapeutic agent. In some embodiments, a PD-L1-binding agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of a PD-L1-binding agent described herein depends on the type of disease to be treated, the severity and course of the disease, the responsiveness of the disease, whether the agent is administered for therapeutic or preventative purposes, previous therapy, the patient's clinical history, and so on, all at the discretion of the treating physician. A PD-L1-binding agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In certain embodiments, dosage is from 0.01 μg to 100 mg/kg of body weight, from 0.1 μg to 100 mg/kg of body weight, from 1 μg to 100 mg/kg of body weight, from lmg to 100 mg/kg of body weight, lmg to 80 mg/kg of body weight from 10 mg to 100 mg/kg of body weight, from 10 mg to 75 mg/kg of body weight, or from 10 mg to 50 mg/kg of body weight. In certain embodiments, the dosage of the agent is from about 0.1 mg to about 20 mg/kg of body weight. In some embodiments, the dosage of the agent is about 0.5 mg/kg of body weight. In some embodiments, the dosage of the agent is about lmg/kg of body weight. In some embodiments, the dosage of the agent is about 1.5 mg/kg of body weight. In some embodiments, the dosage of the agent is about 2 mg/kg of body weight. In some embodiments, the dosage of the agent is about 2.5 mg/kg of body weight. In some embodiments, the dosage of the agent is about 5 mg/kg of body weight. In some embodiments, the dosage of the agent is about 7.5 mg/kg of body weight. In some embodiments, the dosage of the agent is about 10 mg/kg of body weight. In some embodiments, the dosage of the agent is about 12.5 mg/kg of body weight. In some embodiments, the dosage of the agent is about 15 mg/kg of body weight.

In some embodiments, a PD-L1-binding agent may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, a PD-L1-binding agent is administered for 3, 4, 5, 6, 7, 8, or more cycles. For example, a PD-L1-binding agent is administered every 2 weeks for 6 cycles, a PD-L1-binding agent is administered every 3 weeks for 6 cycles, a PD-L1-binding agent is administered every 2 weeks for 4 cycles, a PD-L1-binding agent is administered every 3 weeks for 4 cycles, etc. Dosing schedules can be decided upon and subsequently modified by those skilled in the art.

The present invention provides methods of administering to a subject a PD-L1-binding agent described herein comprising using an intermittent dosing strategy for administering one or more agents, which may reduce side effects and/or toxicities associated with administration of an agent, chemotherapeutic agent, etc. In some embodiments, a method for treating cancer in a human subject comprises administering to the subject a therapeutically effective dose of a PD-L1-binding agent in combination with a therapeutically effective dose of a chemotherapeutic agent, wherein one or both of the agents are administered according to an intermittent dosing strategy. In some embodiments, a method for treating cancer in a human subject comprises administering to the subject a therapeutically effective dose of a PD-L1-binding agent in combination with a therapeutically effective dose of a second immunotherapeutic agent, wherein one or both of the agents are administered according to an intermittent dosing strategy. In some embodiments, the intermittent dosing strategy comprises administering an initial dose of a PD-L1-binding agent to the subject, and administering subsequent doses of the agent about once every 2 weeks. In some embodiments, the intermittent dosing strategy comprises administering an initial dose of a PD-L1-binding agent to the subject, and administering subsequent doses of the agent about once every 3 weeks. In some embodiments, the intermittent dosing strategy comprises administering an initial dose of a PD-L1-binding agent to the subject, and administering subsequent doses of the agent about once every 4 weeks. In some embodiments, a PD-L1-binding agent is administered using an intermittent dosing strategy and the additional therapeutic agent is administered weekly.

The present invention provides compositions comprising a PD-L1-binding agent described herein. The present invention also provides pharmaceutical compositions comprising a PD-L1-binding agent described herein and a pharmaceutically acceptable vehicle. In some embodiments, the pharmaceutical compositions find use in immunotherapy. In some embodiments, the pharmaceutical compositions find use in cancer immunotherapy. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a purified antibody or agent of the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22^{nd} Edition, 2012, Pharmaceutical Press, London.).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The PD-L1-binding agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{nd} Edition, 2012, Pharmaceutical Press, London.

In certain embodiments, pharmaceutical formulations include a PD-L1-binding agent of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising a PD-L1-binding agent described herein can be produced. Suitable examples of sustained-release preparations include semi-permeable matrices of solid hydrophobic polymers containing an agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

### V. Screening

The present invention provides screening methods to identify PD-L1-binding agents that modulate the immune response. In some embodiments, the present invention provides methods for screening candidate agents, including but not limited to, polypeptides, antibodies, peptides, peptidomimetics, small molecules, compounds, or other drugs, which modulate the immune response.

In some embodiments, a method of screening for a candidate PD-L1-binding agent that modulates the immune response comprises determining if the agent has an effect on immune cells. In some embodiments, a method of screening for a candidate PD-L1-binding agent that modulates the immune response comprises determining if the agent is capable of increasing the activity of immune cells. In some embodiments, a method of screening for a candidate PD-L1-binding agent that modulates the immune response comprises determining if the agent is capable of increasing the activity of T-cells. In some embodiments, a method of screening for a candidate PD-L1-binding agent that modulates the immune response comprises determining if the agent is capable of increasing a Th1-type response. In some embodiments, a method of screening for a candidate PD-L1-binding agent that modulates the immune response comprises determining if the agent is capable of decreasing a Th2-type response. In some embodiments, a method of screening for a candidate PD-L1-binding agent that modulates the immune response comprises determining if the agent is capable of increasing the activity of cytolytic cells, such as CTLs and/or NK cells. In some embodiments, a method of screening for a candidate PD-L1-binding agent that modulates the immune response comprises determining if the agent is capable of decreasing the activity of immune suppressor cells, such as Tregs or MDSCs.

### VI. Kits Comprising Agents Described Herein

The present invention provides kits that comprise a PD-L1-binding agent described herein and that can be used to perform the methods described herein. In certain embodiments, a kit comprises at least one purified PD-L1-binding agent in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed PD-L1-binding agents described herein can be readily incorporated into one of the established kit formats which are well known in the art.

Further provided are kits that comprise a PD-L1-binding agent as well as at least one additional therapeutic agent. In certain embodiments, the second (or more) therapeutic agent is a chemotherapeutic agent. In certain embodiments, the second (or more) therapeutic agent is an antibody.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Generation of Anti-PD-L1 Monoclonal Antibodies

Antibodies were generated against recombinant human PD-L1 amino acids 19-241 (SEQ ID NO:3). Mice (n=3) were immunized with PD-L1 using standard techniques. Sera from individual mice were screened against human PD-L1 approximately 70 days after initial immunization using FACS analysis. The animal with the highest antibody titer was selected for a final antigen boost after which spleen cells were isolated for hybridoma production. SP2/0 cells were used as fusion partners for the mouse spleen cells. Hybridoma cells were plated at 1 cell per well in 96 well plates, and the supernatants were screened against human PD-L1 by FACS analysis.

For FACS screening of anti-PD-L1 antibodies a chimeric fusion protein enabling cell surface expression of the extracellular domain of human PD-L1 was constructed (FLAG-hPD-L1-CD4TM-GFP) and transfected into HEK-293 cells. After 48 hours, transfected cells were suspended in ice cold PBS containing 2% FBS and heparin and incubated on ice in the presence of 50 μl of hybridoma supernatants for 30 minutes. A second incubation with 100 μl PE-conjugated anti-human Fc secondary antibody was performed to detect cells bound by antibody. Cells were incubated with an anti-FLAG antibody (Sigma-Aldrich) as a positive control and an anti-PE antibody as a negative control. The cells were analyzed on a FACSCalibur instrument (BD Biosciences) and the data was processed using FlowJo software.

Several hybridomas were identified that bound human PD-L1 and antibody 332M1 was selected. The amino acid sequences of the heavy chain variable region and the light chain variable region of 332M1 are SEQ ID NO:10 and SEQ ID NO:11, respectively. The nucleotide sequences of the heavy chain variable region and the light chain variable region of 332M1 are SEQ ID NO:12 and SEQ ID NO:13, respectively.

The 332M1 antibody was humanized using standard techniques known to those of skill in the art. The humanized version of 332M1 is referred to herein as 332M7. The amino acid sequences of the heavy chain variable region and the light chain variable region of 332M7 are SEQ ID NO:14 and SEQ ID NO:15, respectively. The nucleotide sequences of the heavy chain variable region and the light chain variable region of 332M7 are SEQ ID NO:22 and SEQ ID NO:23, respectively. The heavy chain and light chain CDRs of 332M1/332M7 are listed in Table 1 herein (SEQ ID NOs:4-9). The amino acid sequence of the heavy chain of 332M7 (IgG1 version) with the predicted signal sequence is SEQ ID NO:16 and without a signal sequence is SEQ ID NO:17. The amino acid sequence of the heavy chain of 332M7 (IgG4 version) with the predicted signal sequence is SEQ ID NO:18 and without a signal sequence is SEQ ID NO:19. The amino acid sequence of the light chain of 332M7 with the predicted signal sequence is SEQ ID NO:20 and without a signal sequence is SEQ ID NO:21. The nucleotide sequences of the heavy chain of 332M72 (IgG1 version; with and without signal sequence) are SEQ ID NO:24 and SEQ ID NO:25, of the heavy chain of 332M7 (IgG4 version; with and without signal sequence) are SEQ ID NO:26 and SEQ ID NO:27, and of the light chain (with and without signal sequence) are SEQ ID NO:28 and SEQ ID NO:29.

A plasmid encoding the heavy chain variable region of the 332M7 antibody was deposited with American Type Culture Collection (ATCC), 10801 University Boulevard, Manassas, Va., USA, under the conditions of the Budapest Treaty on Oct. 21, 2015, and designated PTA-122628. A plasmid encoding the light chain of the 332M7 antibody was deposited with ATCC, 10801 University Boulevard, Manassas, Va., USA, under the conditions of the Budapest Treaty on Oct. 21, 2015, and designated PTA-122628.

Antibodies 332M1, 332M7, and 332M8 were further characterized by FACS analysis for binding strength. Antibody 332M8 is a second humanized version of anti-hPD-L1 antibody 332M1. As described above, a hPD-L1-GFP construct was transfected into HEK-293 cells. After 48 hours, transfected cells were suspended in ice cold HBSS containing 2% FBS and incubated on ice in the presence of antibodies 332M1, 332M7, and 332M8 at concentrations of 1, 0.1, and 0.01 μg/ml. A second incubation with 100 μl APC-conjugated anti-mouse or anti-human Fc secondary antibody was performed to detect cells bound by antibody. The cells were analyzed on a FACSCanto instrument (BD Biosciences) and the data was processed using FlowJo software.

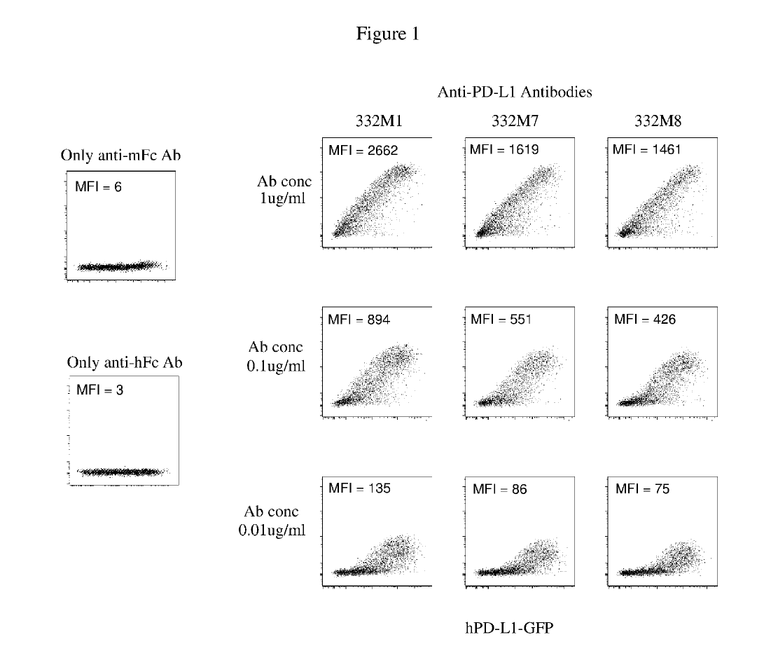

As shown in FIG. 1, antibodies 332M1, 332M7, and 332M8 bound human PD-L1 with similar characteristics.

FACS analysis was done to characterize the binding of antibodies 332M1, 332M7, and 332M8 to mouse PD-L1 (mPD-L1) and cynomolgus PD-L1 (cyno PD-L1). A mPD-L1-CD4TM-GFP construct or a cyno PD-L1-CD4TM-GFP construct was transfected into HEK-293 cells. After 48 hours, transfected cells were suspended in ice cold HBSS containing 2% FBS and incubated on ice in the presence of antibodies 332M1, 332M7, and 332M8 at concentrations of 10 μg/ml. A second incubation with 100 μl APC-conjugated anti-mouse or anti-human Fc secondary antibody was performed to detect cells bound by antibody. The cells were analyzed on a FACSCanto instrument (BD Biosciences) and the data was processed using FlowJo software.

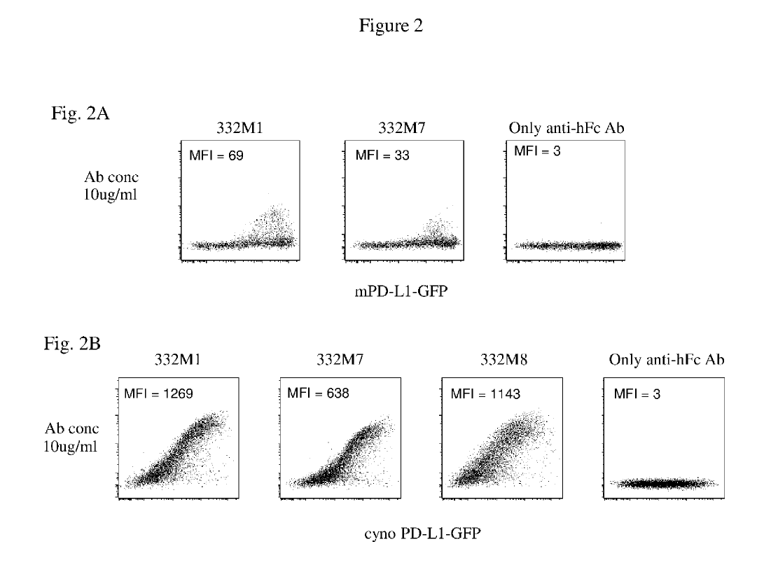

Antibodies 332M1 and 332M7 were observed to have very weak binding to mouse PD-L1 (FIG. 2A). Antibodies 332M1, 332M7, and 323M8 were observed to have strong binding to cynomolgus PD-L1 (FIG. 2B).

### Example 2

### FACS Analysis of Anti-PD-L1 Antibody Blocking of Human PD-L1 to Human PD-1

A cell surface human PD-L1 protein was generated by ligating amino acids 19-241 of human PD-L1 to the transmembrane domain of CD4 and a C-terminal GFP protein tag using standard recombinant DNA techniques (hPD-L1-CD4TM-GFP). PD-1-Fc constructs were generated using standard recombinant DNA techniques. Specifically, the extracellular domain of human PD-1 was ligated in-frame to a mouse Fc region and the recombinant PD-1-Fc protein was expressed in CHO cells. The fusion proteins were purified from cell culture medium using protein A chromatography.

HEK-293T cells were transiently transfected with the hPD-L1-CD4TM-GFP construct. After 48 hours, transfected cells were suspended in ice cold HBSS containing 2% FBS and heparin and incubated on ice with 1 μg/ml PD-1-Fc fusion protein in the presence of anti-PD-L1 antibodies 332M1, 332M7, or 332M8 for 30 minutes. The antibodies were tested at concentrations of 10 and 5 ug/ml. Cells were incubated without antibody or without the PD-1-Fc fusion protein as controls. A second incubation with 100 μl APC-conjugated anti-mouse Fc secondary antibody was performed to detect cells bound by the PD-1-Fc fusion protein. The cells were analyzed on a FACSCanto instrument (BD Biosciences) and the data was processed using FlowJo software.

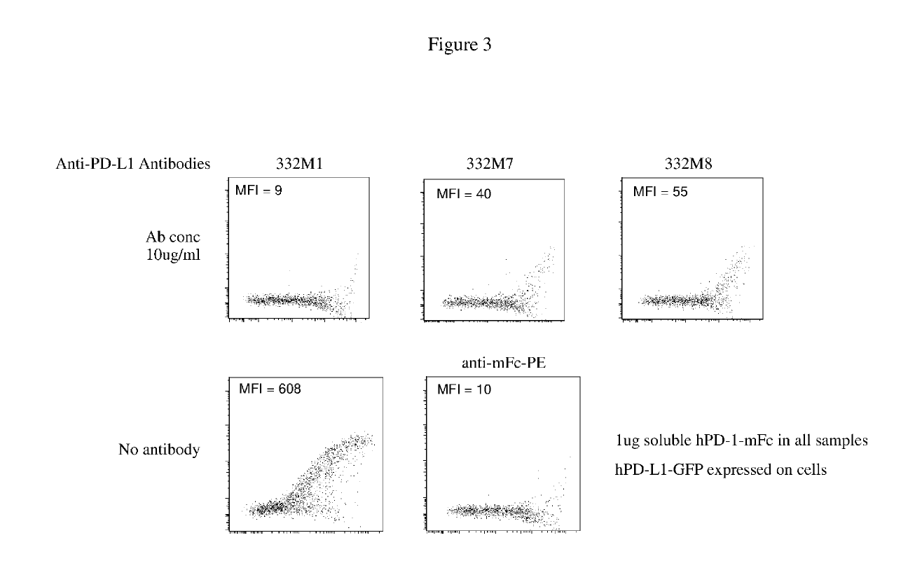

As shown in FIG. 3, in the absence of any anti-hPD-L1 antibody, hPD-1-Fc bound strongly to hPD-L1 expressed on the surface of the HEK-293T cells. All three anti-hPD-L1 antibodies blocked binding of hPD-1-Fc to hPD-L1 at 10 μg/ml.

### Example 3

### In Vivo Tumor Growth Inhibition in Humanized Mice by Anti-PD-L1 Antibody

Humanized mice were obtained from Jackson Laboratories. These mice were created by injecting human hematopoietic stem cells (CD34+ cells) into irradiated NSG mice. After 15 weeks, the presence of mature human lymphocytes was confirmed by flow cytometry. OMP-M9 is a patient-derived melanoma tumor. 75,000 tumor cells per mouse were injected subcutaneously into the humanized mice. Tumors were allowed to grow 16 days until they had reached an average volume of approximately 60 mm³. Tumor-bearing mice were randomized into 2 groups (n=3 mice per group). Tumor-bearing mice were treated with either a control antibody or anti-human PD-L1 antibody OMP-332M1. Antibodies were dosed twice weekly at 10 mg/kg. Tumor volumes were measured on the indicated days post-treatment and shown as the mean plus SEM.

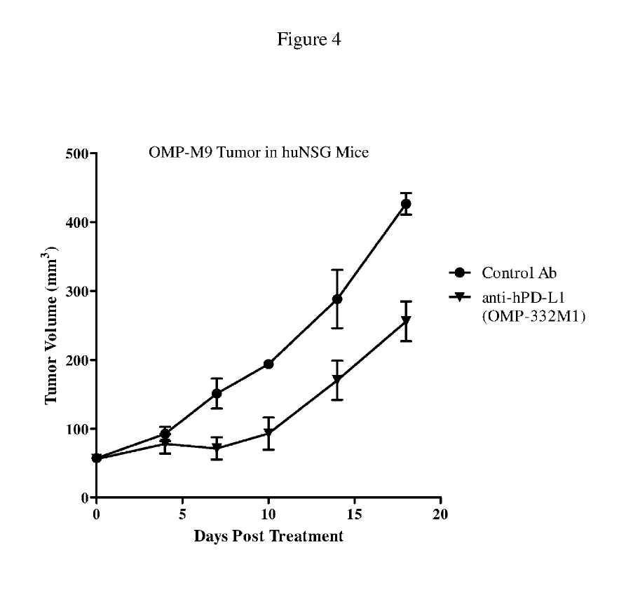

As shown in FIG. 4, anti-hPD-L1 antibody inhibited tumor growth as compared to a control antibody.

Tumors were harvested from the humanized mice described above. For immune response gene expression, quantitative real-time RT-PCR was performed on total RNA obtained from the tumor samples. The tumor samples are expected to contain tumor cells, immune cells associated with the tumor, and any stromal cells attached to the tumor sample. Tumor specimens were harvested and immediately snap frozen and stored at −80° C. prior to RNA isolation. Total RNA was extracted using the RNeasy Fibrous Mini Kit (Qiagen, Valencia Calif.) with TissueLyzer homogenization and DNase I treatment according to the manufacturer's protocol. RNAs were visualized on a Bioanalyzer 2100 (Agilent, Santa Clara, Calif.) and verified to be intact with RIN values >6.0. All RNAs had A260/A280 ratios >1.8.

cDNA was synthesized from total RNA using random hexamers. The cDNA and PCR Master Mix were added to a TaqMan Array Immune Response Plate (Applied Biosystems/Life Technologies) and reactions were run on a real-time PCR instrument according to the manufacturer's protocol.

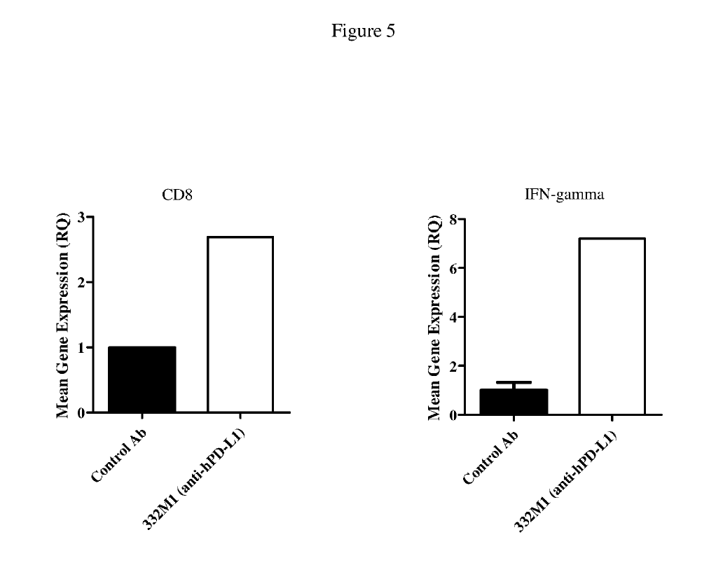

As shown in FIG. 5, gene expression of CD8 and IFN-γ was increased in tumor samples from mice treated with anti-PD-L1 antibody as compared to gene expression in tumor samples from mice treated with the control antibody. These results suggest that the number of CD8+ cells within the tumor or tumor microenvironment was increased. In addition, the results suggest that the CD8+ cells and/or other activated immune cells were producing increased levels of IFN-γ after treatment with the anti-PD-L1 antibody.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

The following sequences are disclosed in the application:

Human PD-L1 amino acid sequence
(SEQ ID NO: 1)
MRIFAVFIFMTYWHLLNAFTVTVPKDLYVVEYGSNMTIECKFPVEKQLDLAALIVYWEME
DKNIIQFVHGEEDLKVQHSSYRQRARLLKDQLSLGNAALQITDVKLQDAGVYRCMISYGG
ADYKRITVKVNAPYNKINQRILVVDPVTSEHELTCQAEGYPKAEVIWTSSDHQVLSGKTT
TTNSKREEKLFNVTSTLRINTTTNEIFYCTFRRLDPEENHTAELVIPELPLAHPPNERTH
LVILGAILLCLGVALTFIFRLRKGRMMDVKKCGIQDTNSKKQSDTHLEET
Human PD-L1 amino acid sequence without predicted signal sequence
(SEQ ID NO: 2)
FTVTVPKDLYVVEYGSNMTIECKFPVEKQLDLAALIVYWEMEDKNIIQFVHGEEDLKVQH
SSYRQRARLLKDQLSLGNAALQITDVKLQDAGVYRCMISYGGADYKRITVKVNAPYNKIN
QRILVVDPVTSEHELTCQAEGYPKAEVIWTSSDHQVLSGKTTTTNSKREEKLFNVTSTLR
INTTTNEIFYCTFRRLDPEENHTAELVIPELPLAHPPNERTHLVILGAILLCLGVALTFI
FRLRKGRMMDVKKCGIQDTNSKKQSDTHLEET
Human PD-L1 extracellular domain amino acid sequence
(SEQ ID NO: 3)
FTVTVPKDLYVVEYGSNMTIECKFPVEKQLDLAALIVYWEMEDKNIIQFVHGEEDLKVQH
SSYRQRARLLKDQLSLGNAALQITDVKLQDAGVYRCMISYGGADYKRITVKVNAPYNKIN
QRILVVDPVTSEHELTCQAEGYPKAEVIWTSSDHQVLSGKTTTTNSKREEKLFNVTSTLR
INTTTNEIFYCTFRRLDPEENHTAELVIPELPLAHPPNERTHL
332M1/332M7 Heavy chain CDR1
(SEQ ID NO: 4)
TSYWMH
332M1/332M7 Heavy chain CDR2
(SEQ ID NO: 5)
AIYPGNSDTSYNQKFKG
332M1/332M7 Heavy chain CDR3
(SEQ ID NO: 6)
WGYGFDGAMDY
332M1/332M7 Light chain CDR1
(SEQ ID NO: 7)
RASQDIGSSLN
332M1/332M7 Light chain CDR2
(SEQ ID NO: 8)
ATSSLDS
332M1/332M7 Light chain CDR3
(SEQ ID NO: 9)
LQYASSP
332M1 Heavy chain variable region amino acid sequence
(SEQ ID NO: 10)
QVQLQQSGPELARPGASVKMSCKASGYSFTSYWMHWVKQRPGQGLEWIGAIYPGNSDTSY
NQKFKGKAKLTAVTSASTAYMELSSLTNEDSAVYYCTRWGYGFDGAMDYWGQGTSVTVSS
332M1 Light chain variable region amino acid sequence
(SEQ ID NO: 11)
DIVTQSPSSLSASLGERVSLTCRASQDIGSSLNWLQQEPDGTIKRLIYATSSLDSGVPKR
FSGSRSGSDYSLTISSLESEDFVDYYCLQYASSPYTFGGGTKLEIKR
332M1 Heavy chain variable region nucleotide sequence
(SEQ ID NO: 12)
CAAGTCCAATTGCAGCAGTCTGGACCTGAGCTGGCAAGGCCTGGGGCTTCCGTGAAGATG
TCCTGCAAGGCTTCTGGCTACAGCTTTACCAGCTACTGGATGCACTGGGTAAAACAGAGG
CCTGGACAGGGTCTAGAATGGATTGGTGCTATTTATCCTGGAAATAGTGATACTAGCTAC
AACCAGAAGTTCAAGGGCAAGGCCAAGCTGACTGCAGTCACATCCGCCAGCACTGCCTAC
ATGGAGCTCAGCAGCCTGACAAATGAGGACTCTGCGGTCTATTACTGTACAAGATGGGGG
TATGGGTTCGACGGAGCTATGGACTACTGGGGTCAAGGAACCTCAGTCACCGTCTCCTCA
332M1 Light chain variable region nucleotide sequence
(SEQ ID NO: 13)
GATATCGTGACCCAGTCTCCATCCTCCTTATCTGCCTCTCTGGGAGAAAGAGTCAGTCTC
ACTTGTCGGGCAAGTCAGGACATTGGTAGTAGCTTAAACTGGCTTCAGCAGGAACCAGAT
GGAACTATTAAACGCCTGATCTACGCCACATCCAGTTTAGATTCTGGTGTCCCCAAAAGG
TTCAGTGGCAGTAGGTCTGGGTCAGATTATTCTCTCACCATCAGCAGCCTTGAGTCTGAA
GATTTTGTAGACTATTACTGTCTACAATATGCTAGTTCTCCGTACACGTTCGGAGGGGGG
ACCAAGCTGGAAATAAAACGG
332M7 Heavy chain variable region amino acid sequence
(SEQ ID NO: 14)
QVQLVQSGAEVKKPGASVKVSCKASGYTFTSYWMHWVRQAPGQGLEWMGAIYPGNSDTSY
NQKFKGRVTMTRDTSTSTVYMELSSLRSEDTAVYYCTRWGYGFDGAMDYWGQGTLVTVSS
332M7 Light chain variable region amino acid sequence
(SEQ ID NO: 15)
DIQMTQSPSSLSASVGDRVTITCRASQDIGSSLNWYQQKPGKAPKRLIYATSSLDSGVPS
RFSGSGSGTEFTLTISSLQPEDFATYYCLQYASSPYTFGGGTKVEIKR
332M7 Heavy chain (IgG1) amino acid sequence with predicted signal
sequence underlined
(SEQ ID NO: 16)
MDWTWRILFLVAAATGAHSQVQLVQSGAEVKKPGASVKVSCKASGYTFTSYWMHWVRQAP
GQGLEWMGAIYPGNSDTSYNQKFKGRVTMTRDTSTSTVYMELSSLRSEDTAVYYCTRWGY
GFDGAMDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
332M7 Heavy chain (IgG1) amino acid sequence without signal sequence
(SEQ ID NO: 17)
QVQLVQSGAEVKKPGASVKVSCKASGYTFTSYWMHWVRQAPGQGLEWMGAIYPGNSDTSY
NQKFKGRVTMTRDTSTSTVYMELSSLRSEDTAVYYCTRWGYGFDGAMDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
332M7 Heavy chain (IgG4) amino acid sequence with predicted signal sequence
underlined
(SEQ ID NO: 18)
MDWTWRILFLVAAATGAHSQVQLVQSGAEVKKPGASVKVSCKASGYTFTSYWMHWVRQAP
GQGLEWMGAIYPGNSDTSYNQKFKGRVTMTRDTSTSTVYMELSSLRSEDTAVYYCTRWGY
GFDGAMDYWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESK
YGPPCPPCPAPEFLGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
332M7 Heavy chain (IgG4) amino acid sequence without signal sequence
(SEQ ID NO: 19)
QVQLVQSGAEVKKPGASVKVSCKASGYTFTSYWMHWVRQAPGQGLEWMGAIYPGNSDTSY
NQKFKGRVTMTRDTSTSTVYMELSSLRSEDTAVYYCTRWGYGFDGAMDYWGQGTLVTVSS
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
332M7 Light chain amino acid sequence with predicted signal sequence
underlined
(SEQ ID NO: 20)
MVLQTQVFISLLLWISGAYGDIQMTQSPSSLSASVGDRVTITCRASQDIGSSLNWYQQKP
GKAPKRLIYATSSLDSGVPSRFSGSGSGTEFTLTISSLQPEDFATYYCLQYASSPYTFGG
GTKVEIKRTVAAPSVFIFPPSDEQLKSGTASVVCLLNNFYPREAKVQWKVDNALQSGNSQ
ESVTEQDSKDSTYSLSNTLTLSKADYEKHKVYACEVTHQGLSSPVTKSFNRGEC
332M7 Light chain amino acid sequence without signal sequence
(SEQ ID NO: 21)
DIQMTQSPSSLSASVGDRVTITCRASQDIGSSLNWYQQKPGKAPKRLIYATSSLDSGVPS
RFSGSGSGTEFTLTISSLQPEDFATYYCLQYASSPYTFGGGTKVEIKRTVAAPSVFIFPP
SDEQLKSGTASVVCLLNNFYPREAKVQWKVDNALQSGNSQESVTEQDSKDSTYSLSNTLT
LSKADYEKHKVYACEVTHQGLSSPVTKSFNRGEC
332M7 Heavy chain variable region nucleotide sequence
(SEQ ID NO: 22)
GACATCCAGATGACCCAGTCTCCATCCTCCCTGTCTGCATCTGTCGGAGACAGAGTCACC
ATCACTTGCCGGGCATCTCAGGACATTGGTTCCTCTCTCAACTGGTATCAGCAGAAACCA
GGGAAAGCCCCTAAGCGCCTGATCTATGCCACATCCTCTCTGGATTCTGGGGTCCCATCA
AGGTTCAGCGGCTCCGGATCTGGGACAGAATTTACTCTCACAATCAGCAGCCTGCAGCCT
GAAGATTTTGCAACTTATTACTGTCTCCAATATGCTTCTTCTCCTTACACTTTCGGCGGA
GGGACCAAGGTGGAGATCAAACGT
332M7 Light chain variable region nucleotide sequence
(SEQ ID NO: 23)
GACATCCAGATGACCCAGTCTCCATCCTCCCTGTCTGCATCTGTCGGAGACAGAGTCACC
ATCACTTGCCGGGCATCTCAGGACATTGGTTCCTCTCTCAACTGGTATCAGCAGAAACCA
GGGAAAGCCCCTAAGCGCCTGATCTATGCCACATCCTCTCTGGATTCTGGGGTCCCATCA
AGGTTCAGCGGCTCCGGATCTGGGACAGAATTTACTCTCACAATCAGCAGCCTGCAGCCT
GAAGATTTTGCAACTTATTACTGTCTCCAATATGCTTCTTCTCCTTACACTTTCGGCGGA
GGGACCAAGGTGGAGATCAAACGT
332M7 Heavy chain (IgG1) nucleotide sequence with signal sequence
(SEQ ID NO: 24)
ATGGACTGGACCTGGAGGATACTCTTTCTCGTGGCTGCAGCCACAGGAGCCCACTCCCAA
GTCCAGCTGGTGCAGTCTGGGGCTGAGGTGAAGAAGCCTGGGGCCTCAGTGAAGGTTTCC
TGCAAGGCATCTGGATACACCTTCACCAGCTACTGGATGCACTGGGTGCGGCAGGCCCCT
GGACAAGGGCTCGAATGGATGGGAGCTATTTATCCTGGAAATTCCGATACTAGCTACAAC
CAGAAGTTCAAGGGCAGAGTCACCATGACCAGGGACACATCCACTAGCACAGTCTACATG
GAGCTGTCTAGCCTGCGGTCTGAGGACACTGCCGTGTATTACTGTACAAGATGGGGGTAT
GGGTTCGACGGAGCTATGGACTACTGGGGCCAGGGCACCCTGGTCACCGTCAGCTCAGCC
AGCACAAAGGGCCCCTCCGTGTTCCCTCTGGCCCCTTCCTCCAAGTCCACCTCCGGCGGC
ACCGCCGCTCTGGGCTGCCTGGTGAAGGACTACTTCCCTGAGCCTGTGACCGTGTCCTGG
AACTCTGGCGCCCTGACCTCTGGCGTGCACACCTTCCCAGCCGTGCTGCAGTCCTCCGGC
CTGTACTCCCTGTCCTCCGTGGTGACCGTGCCTTCCTCCTCCCTGGGCACCCAGACCTAC
ATCTGCAACGTGAACCACAAGCCTTCCAACACCAAGGTGGACAAGCGGGTGGAGCCTAAG
TCCTGCGACAAGACCCACACCTGCCCTCCCTGCCCTGCCCCTGAGCTGCTGGGCGGACCT
TCCGTGTTCCTGTTCCCTCCTAAGCCTAAGGACACCCTGATGATCTCCCGGACCCCTGAG
GTGACCTGCGTGGTGGTGGACGTGTCCCACGAGGATCCTGAGGTGAAGTTCAATTGGTAC
GTGGACGGCGTGGAGGTGCACAACGCTAAGACCAAGCCAAGGGAGGAGCAGTACAACTCC
ACCTACCGGGTGGTGTCTGTGCTGACCGTGCTGCACCAGGACTGGCTGAACGGCAAAGAA
TACAAGTGCAAGGTCTCCAACAAGGCCCTGCCCGCTCCCATCGAGAAAACCATCTCCAAG
GCCAAGGGCCAGCCTCGCGAGCCTCAGGTGTACACCCTGCCACCCAGCCGGGAGGAGATG
ACCAAGAACCAGGTGTCCCTGACCTGTCTGGTGAAGGGCTTCTACCCTTCCGATATCGCC
GTGGAGTGGGAGTCTAACGGCCAGCCCGAGAACAACTACAAGACCACCCCTCCTGTGCTG
GACTCCGACGGCTCCTTCTTCCTGTACTCCAAGCTGACCGTGGACAAGTCCCGGTGGCAG
CAGGGCAACGTGTTCTCCTGCTCCGTGATGCACGAGGCCCTGCACAACCACTACACCCAG
AAGAGCCTGTCTCTGTCTCCTGGCAAGTGA
332M7 Heavy chain (IgG1) nucleotide sequence without signal sequence
(SEQ ID NO: 25)
CAAGTCCAGCTGGTGCAGTCTGGGGCTGAGGTGAAGAAGCCTGGGGCCTCAGTGAAGGTT
TCCTGCAAGGCATCTGGATACACCTTCACCAGCTACTGGATGCACTGGGTGCGGCAGGCC
CCTGGACAAGGGCTCGAATGGATGGGAGCTATTTATCCTGGAAATTCCGATACTAGCTAC
AACCAGAAGTTCAAGGGCAGAGTCACCATGACCAGGGACACATCCACTAGCACAGTCTAC
ATGGAGCTGTCTAGCCTGCGGTCTGAGGACACTGCCGTGTATTACTGTACAAGATGGGGG
TATGGGTTCGACGGAGCTATGGACTACTGGGGCCAGGGCACCCTGGTCACCGTCAGCTCA
GCCAGCACAAAGGGCCCCTCCGTGTTCCCTCTGGCCCCTTCCTCCAAGTCCACCTCCGGC
GGCACCGCCGCTCTGGGCTGCCTGGTGAAGGACTACTTCCCTGAGCCTGTGACCGTGTCC
TGGAACTCTGGCGCCCTGACCTCTGGCGTGCACACCTTCCCAGCCGTGCTGCAGTCCTCC
GGCCTGTACTCCCTGTCCTCCGTGGTGACCGTGCCTTCCTCCTCCCTGGGCACCCAGACC
TACATCTGCAACGTGAACCACAAGCCTTCCAACACCAAGGTGGACAAGCGGGTGGAGCCT
AAGTCCTGCGACAAGACCCACACCTGCCCTCCCTGCCCTGCCCCTGAGCTGCTGGGCGGA
CCTTCCGTGTTCCTGTTCCCTCCTAAGCCTAAGGACACCCTGATGATCTCCCGGACCCCT
GAGGTGACCTGCGTGGTGGTGGACGTGTCCCACGAGGATCCTGAGGTGAAGTTCAATTGG
TACGTGGACGGCGTGGAGGTGCACAACGCTAAGACCAAGCCAAGGGAGGAGCAGTACAAC
TCCACCTACCGGGTGGTGTCTGTGCTGACCGTGCTGCACCAGGACTGGCTGAACGGCAAA
GAATACAAGTGCAAGGTCTCCAACAAGGCCCTGCCCGCTCCCATCGAGAAAACCATCTCC
AAGGCCAAGGGCCAGCCTCGCGAGCCTCAGGTGTACACCCTGCCACCCAGCCGGGAGGAG
ATGACCAAGAACCAGGTGTCCCTGACCTGTCTGGTGAAGGGCTTCTACCCTTCCGATATC
GCCGTGGAGTGGGAGTCTAACGGCCAGCCCGAGAACAACTACAAGACCACCCCTCCTGTG
CTGGACTCCGACGGCTCCTTCTTCCTGTACTCCAAGCTGACCGTGGACAAGTCCCGGTGG
CAGCAGGGCAACGTGTTCTCCTGCTCCGTGATGCACGAGGCCCTGCACAACCACTACACC
CAGAAGAGCCTGTCTCTGTCTCCTGGCAAGTGA
332M7 Heavy chain (IgG4) nucleotide sequence with signal sequence
(SEQ ID NO: 26)
ATGGACTGGACCTGGAGGATACTCTTTCTCGTGGCTGCAGCCACAGGAGCCCACTCCCAA
GTCCAGCTGGTGCAGTCTGGGGCTGAGGTGAAGAAGCCTGGGGCCTCAGTGAAGGTTTCC
TGCAAGGCATCTGGATACACCTTCACCAGCTACTGGATGCACTGGGTGCGGCAGGCCCCT
GGACAAGGGCTCGAATGGATGGGAGCTATTTATCCTGGAAATTCCGATACTAGCTACAAC
CAGAAGTTCAAGGGCAGAGTCACCATGACCAGGGACACATCCACTAGCACAGTCTACATG
GAGCTGTCTAGCCTGCGGTCTGAGGACACTGCCGTGTATTACTGTACAAGATGGGGGTAT
GGGTTCGACGGAGCTATGGACTACTGGGGCCAGGGCACCCTGGTCACCGTCAGCTCAGCC
AGCACAAAGGGCCCATCCGTCTTCCCCCTGGCACCCTGCTCCCGGAGCACCTCCGAGAGC
ACAGCCGCCCTGGGCTGCCTGGTCAAGGACTACTTCCCCGAACCCGTTACCGTGTCTTGG
AACTCCGGCGCACTGACCAGCGGCGTGCACACCTTCCCTGCTGTCCTCCAATCCTCTGGA
CTCTACTCCCTCTCCTCCGTGGTGACAGTGCCCTCCAGCAGCCTGGGCACTAAGACCTAC
ACCTGCAACGTCGATCACAAGCCCAGCAACACCAAGGTGGACAAGAGAGTTGAGTCCAAA
TATGGACCCCCATGCCCACCTTGCCCAGCACCTGAGTTCCTGGGGGGACCATCAGTCTTC
CTGTTCCCCCCAAAACCCAAGGACACTCTCATGATCTCCCGGACCCCTGAGGTCACTTGC
GTGGTGGTGGACGTGAGCCAGGAAGACCCCGAGGTCCAGTTCAACTGGTATGTGGATGGC
GTGGAGGTTCATAATGCCAAGACAAAGCCTCGGGAGGAGCAGTTCAACAGCACCTACCGG
GTGGTCAGCGTCCTCACCGTCCTGCACCAAGACTGGCTGAACGGCAAGGAGTACAAGTGC
AAGGTCTCCAACAAAGGGCTCCCATCCTCCATCGAGAAAACCATCTCCAAAGCCAAAGGG
CAGCCCCGGGAGCCACAGGTGTACACCCTGCCCCCATCCCAAGAGGAGATGACCAAGAAC
CAAGTGTCCCTGACCTGCCTGGTCAAAGGCTTCTACCCCAGCGACATCGCCGTGGAGTGG
GAGAGCAATGGGCAGCCTGAGAACAACTACAAGACCACTCCTCCCGTGCTGGACTCCGAC
GGCTCCTTCTTCCTCTACTCCCGGCTCACCGTGGACAAGAGCAGGTGGCAGGAGGGCAAT
GTCTTCTCCTGCTCCGTGATGCATGAGGCTCTGCACAACCACTACACACAGAAGAGCCTC
TCCCTGTCTCTGGGCAAATGA
332M7 Heavy chain (IgG4) nucleotide sequence without signal sequence
(SEQ ID NO: 27)
CAAGTCCAGCTGGTGCAGTCTGGGGCTGAGGTGAAGAAGCCTGGGGCCTCAGTGAAGGTT
TCCTGCAAGGCATCTGGATACACCTTCACCAGCTACTGGATGCACTGGGTGCGGCAGGCC
CCTGGACAAGGGCTCGAATGGATGGGAGCTATTTATCCTGGAAATTCCGATACTAGCTAC
AACCAGAAGTTCAAGGGCAGAGTCACCATGACCAGGGACACATCCACTAGCACAGTCTAC
ATGGAGCTGTCTAGCCTGCGGTCTGAGGACACTGCCGTGTATTACTGTACAAGATGGGGG
TATGGGTTCGACGGAGCTATGGACTACTGGGGCCAGGGCACCCTGGTCACCGTCAGCTCA
GCCAGCACAAAGGGCCCATCCGTCTTCCCCCTGGCACCCTGCTCCCGGAGCACCTCCGAG
AGCACAGCCGCCCTGGGCTGCCTGGTCAAGGACTACTTCCCCGAACCCGTTACCGTGTCT
TGGAACTCCGGCGCACTGACCAGCGGCGTGCACACCTTCCCTGCTGTCCTCCAATCCTCT
GGACTCTACTCCCTCTCCTCCGTGGTGACAGTGCCCTCCAGCAGCCTGGGCACTAAGACC
TACACCTGCAACGTCGATCACAAGCCCAGCAACACCAAGGTGGACAAGAGAGTTGAGTCC
AAATATGGACCCCCATGCCCACCTTGCCCAGCACCTGAGTTCCTGGGGGGACCATCAGTC
TTCCTGTTCCCCCCAAAACCCAAGGACACTCTCATGATCTCCCGGACCCCTGAGGTCACT
TGCGTGGTGGTGGACGTGAGCCAGGAAGACCCCGAGGTCCAGTTCAACTGGTATGTGGAT
GGCGTGGAGGTTCATAATGCCAAGACAAAGCCTCGGGAGGAGCAGTTCAACAGCACCTAC
CGGGTGGTCAGCGTCCTCACCGTCCTGCACCAAGACTGGCTGAACGGCAAGGAGTACAAG
TGCAAGGTCTCCAACAAAGGGCTCCCATCCTCCATCGAGAAAACCATCTCCAAAGCCAAA
GGGCAGCCCCGGGAGCCACAGGTGTACACCCTGCCCCCATCCCAAGAGGAGATGACCAAG
AACCAAGTGTCCCTGACCTGCCTGGTCAAAGGCTTCTACCCCAGCGACATCGCCGTGGAG
TGGGAGAGCAATGGGCAGCCTGAGAACAACTACAAGACCACTCCTCCCGTGCTGGACTCC
GACGGCTCCTTCTTCCTCTACTCCCGGCTCACCGTGGACAAGAGCAGGTGGCAGGAGGGC
AATGTCTTCTCCTGCTCCGTGATGCATGAGGCTCTGCACAACCACTACACACAGAAGAGC
CTCTCCCTGTCTCTGGGCAAATGA
332M7 Light chain nucleotide sequence with signal sequence
(SEQ ID NO: 28)
ATGGACTGGACCTGGAGGATACTCTTTCTCGTGGCTGCAGCCACAGGAGCCCACTCCGAC
ATCCAGATGACCCAGTCTCCATCCTCCCTGTCTGCATCTGTCGGAGACAGAGTCACCATC
ACTTGCCGGGCATCTCAGGACATTGGTTCCTCTCTCAACTGGTATCAGCAGAAACCAGGG
AAAGCCCCTAAGCGCCTGATCTATGCCACATCCTCTCTGGATTCTGGGGTCCCATCAAGG
TTCAGCGGCTCCGGATCTGGGACAGAATTTACTCTCACAATCAGCAGCCTGCAGCCTGAA
GATTTTGCAACTTATTACTGTCTCCAATATGCTTCTTCTCCTTACACTTTCGGCGGAGGG
ACCAAGGTGGAGATCAAACGTACGGTGGCTGCACCATCTGTCTTCATCTTCCCTCCATCT
GATGAGCAGCTCAAATCTGGAACTGCCTCTGTTGTGTGCCTGCTGAATAACTTCTATCCC
AGAGAGGCCAAAGTCCAGTGGAAGGTGGATAACGCCCTCCAATCCGGCAACTCCCAGGAG
AGTGTCACAGAGCAGGACAGCAAGGACAGCACCTACAGCCTCAGCAACACCCTGACACTG
AGCAAAGCAGACTACGAGAAACACAAAGTCTATGCCTGCGAAGTCACCCATCAGGGCCTG
TCTTCCCCCGTCACAAAGAGCTTCAACAGGGGAGAGTGCTAA
332M7 Light chain nucleotide sequence without signal sequence
(SEQ ID NO: 29)
ATCCAGATGACCCAGTCTCCATCCTCCCTGTCTGCATCTGTCGGAGACAGAGTCACCATC
ACTTGCCGGGCATCTCAGGACATTGGTTCCTCTCTCAACTGGTATCAGCAGAAACCAGGG
AAAGCCCCTAAGCGCCTGATCTATGCCACATCCTCTCTGGATTCTGGGGTCCCATCAAGG
TTCAGCGGCTCCGGATCTGGGACAGAATTTACTCTCACAATCAGCAGCCTGCAGCCTGAA
GATTTTGCAACTTATTACTGTCTCCAATATGCTTCTTCTCCTTACACTTTCGGCGGAGGG
ACCAAGGTGGAGATCAAACGTACGGTGGCTGCACCATCTGTCTTCATCTTCCCTCCATCT
GATGAGCAGCTCAAATCTGGAACTGCCTCTGTTGTGTGCCTGCTGAATAACTTCTATCCC
AGAGAGGCCAAAGTCCAGTGGAAGGTGGATAACGCCCTCCAATCCGGCAACTCCCAGGAG
AGTGTCACAGAGCAGGACAGCAAGGACAGCACCTACAGCCTCAGCAACACCCTGACACTG
AGCAAAGCAGACTACGAGAAACACAAAGTCTATGCCTGCGAAGTCACCCATCAGGGCCTG
TCTTCCCCCGTCACAAAGAGCTTCAACAGGGGAGAGTGCTAA
Human IgG1 Heavy chain constant region
(SEQ ID NO: 30)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG2 Heavy chain constant region
(SEQ ID NO: 31)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGN
VFSCSVMHEALHNHYTQKSLSLSPGK
Human IgG3 Heavy chain constant region
(SEQ ID NO: 32)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYNSTFRVVSVLTVLH
QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE
ALHNRFTQKSLSLSPGK
Human IgG4 Heavy chain constant region
(SEQ ID NO: 33)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
Human IgG4 Heavy chain constant region with stabilized hinge region
(SEQ ID NO: 34)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK

What is claimed is:

1. An isolated antibody that specifically binds the extracellular domain of human PD-L1, which comprises:
(a) a heavy chain CDR1 comprising TSYWMH (SEQ ID NO:4), a heavy chain CDR2 comprising AIYPGNSDTSYNQKFKG (SEQ ID NO:5), and a heavy chain CDR3 comprising WGYGFDGAMDY (SEQ ID NO:6), and/or
(b) a light chain CDR1 comprising RASQDIGSSLN (SEQ ID NO:7), a light chain CDR2 comprising ATSSLDS (SEQ ID NO:8), and a light chain CDR3 comprising LQYASSP (SEQ ID NO:9).

2. An isolated antibody that specifically binds human PD-L1, which comprises:
(a) a heavy chain variable region having at least 90% sequence identity to SEQ ID NO:10 or SEQ ID NO:14; and/or
(b) a light chain variable region having at least 90% sequence identity to SEQ ID NO:11 or SEQ ID NO:15.

3. The antibody of claim 1 or claim 2, which comprises:
(a) a heavy chain variable region having at least 95% sequence identity to SEQ ID NO:10 or SEQ ID NO:14; and/or
(b) a light chain variable region having at least 95% sequence identity to SEQ ID NO:11 or SEQ ID NO:15.

4. The antibody of claim 3, which comprises a heavy chain variable region comprising SEQ ID NO:10 and a light chain variable region comprising SEQ ID NO:11.

5. The antibody of claim 3, which comprises a heavy chain variable region comprising SEQ ID NO:14 and a light chain variable region comprising SEQ ID NO:15.

6. The antibody of any one of claims 1-5, which is a monoclonal antibody.

7. The antibody of any one of claim 1-3, 5, or 6, which is a humanized antibody.

8. The antibody of claim 1, which is a human antibody.

9. The antibody of any one of claims 1-8, which is a recombinant antibody or a chimeric antibody.

10. The antibody of any one of claims 1-9, which is a bispecific antibody.

11. The antibody of any one of claims 1-10, which is an antibody fragment comprising an antigen binding site.

12. The antibody of any one of claims 1-11, which is an IgG antibody.

13. The antibody of claim 12, which is an IgG1 antibody, an IgG2 antibody, or an IgG4 antibody.

14. An antibody comprising a heavy chain amino acid sequence of SEQ ID NO:17 or SEQ ID NO:19 and a light chain amino acid sequence of SEQ ID NO:21.

15. An antibody comprising the same heavy chain variable region and the light chain variable region amino acid sequences as antibody 332M7.

16. An antibody comprising the heavy chain variable region encoded by the plasmid deposited with ATCC as PTA-122627.

17. An antibody comprising the light chain variable region encoded by the plasmid deposited with ATCC as PTA-122628.

18. An antibody comprising the light chain encoded by the plasmid deposited with ATCC as PTA-122628.

19. An antibody comprising the heavy chain variable region encoded by the plasmid deposited with ATCC as PTA-122627 and the light chain variable region encoded by the plasmid deposited with ATCC as PTA-122628.

20. An antibody comprising the heavy chain variable region encoded by the plasmid deposited with ATCC as PTA-122627 and the light chain encoded by the plasmid deposited with ATCC as PTA-122628.

21. An isolated antibody that competes with the antibody of any one of claims 1-20 for specific binding to PD-L1.

22. An isolated antibody that binds the same epitope on PD-L1 as the antibody of any one of claims 1-20.

23. An isolated antibody that binds an epitope on PD-L1 that overlaps with the epitope on PD-L1 bound by the antibody of any one of claims 1-20.

24. The antibody of any one of claims 1-23, which inhibits binding of PD-L1 to PD-1.

25. The antibody of any one of claims 1-23, which inhibits or blocks the interaction between PD-L1 and PD-1.

26. The antibody of any one of claims 1-23, which inhibits binding of PD-L1 to CD80.

27. The antibody of any one of claims 1-23, which inhibits or blocks the interaction between PD-L1 and CD80.

28. The antibody of any one of claims 1-23, which inhibits PD-L1 signaling.

29. The antibody of any one of claims 1-23, which is an antagonist of PD-L1-mediated signaling.

30. The antibody of any one of claims 1-23, which inhibits PD-L1-mediated PD-1 activity.

31. The antibody of any one of claims 1-23, which inhibits PD-L1-mediated CD80 activity.

32. The antibody of any one of claims 1-23, which induces and/or enhances an immune response.

33. The antibody of claim 32, wherein the immune response is directed to a tumor or tumor cell.

34. The antibody of any one of claims 1-23, which increases cell-mediated immunity.

35. The antibody of any one of claims 1-23, which increases T-cell activity.

36. The antibody of any one of claims 1-23, which increases cytolytic T-cell (CTL) activity.

37. The antibody of any one of claims 1-23, which increases natural killer (NK) cell activity.

38. The antibody of any one of claims 1-23, which increases IL-2 production and/or the number of IL-2-producing cells.

39. The antibody of any one of claims 1-23, which increases IFN-gamma production and/or the number of IFN-gamma-producing cells.

40. The antibody of any one of claims 1-23, which increases a Th1-type immune response.

41. The antibody of any one of claims 1-23, which decreases IL-4 production and/or the number of IL-4-producing cells.

42. The antibody of any one of claims 1-23, which decreases IL-10 and/or the number of IL-10-producing cells.

43. The antibody of any one of claims 1-23, which decreases a Th2-type immune response.

44. The antibody of any one of claims 1-23, which inhibits and/or decreases the suppressive activity of regulatory T-cells (Tregs).

45. The antibody of any one of claims 1-23, which inhibits and/or decreases the suppressive activity of myeloid-derived suppressor cells (MDSCs).

46. The antibody of any one of claims 1-45, which inhibits tumor growth.

47. A heterodimeric agent comprising the antibody of any one of claims 1-20.

48. A bispecific agent comprising:
a) a first arm that specifically binds PD-L1, and
b) a second arm,
wherein the first arm comprises an antibody of any one of claims 1-21.

49. The bispecific agent of claim 48, wherein the second arm comprises an antigen-binding site from an antibody.

50. The bispecific agent of claim 48, wherein the second arm specifically binds PD-1, TIGIT, CTLA-4, TIM-3, LAG-3, OX-40, CD40, or GITR.

51. The bispecific agent of claim 48, wherein the second arm specifically binds a tumor antigen.

52. The bispecific agent of claim 48, wherein the second arm comprises an immunotherapeutic agent.

53. The bispecific agent of claim 52, wherein the immunotherapeutic agent is selected from the group consisting of: granulocyte-macrophage colony stimulating factor (GM-CSF), macrophage colony stimulating factor (M-CSF), granulocyte colony stimulating factor (G-CSF), interleukin 2 (IL-2), interleukin 3 (IL-3), interleukin 12 (IL-12), interleukin 15 (IL-15), B7-1 (CD80), B7-2 (CD86), 4-1BB ligand, GITRL, CD40L, OX40L, anti-CD3 antibody, anti-CTLA4 antibody, anti-PD-1 antibody, anti-TIGIT antibody, anti-GITR antibody, anti-OX40 antibody, anti-CD40 antibody, anti-4-1BB antibody, anti-LAG-3 antibody, and anti-TIM-3 antibody.

54. The bispecific agent of any one of claims 48-53, wherein the first arm comprises a first CH3 domain and the second arm comprises a second CH3 domain, each of which is modified to promote formation of heterodimers.

55. The bispecific agent of claim 54, wherein the first and second CH3 domains are modified based upon electrostatic effects.

56. The bispecific agent of any one of claims 48-53, wherein the first arm comprises a first human IgG1 constant region with amino acid substitutions at positions corresponding to positions 253 and 292 of SEQ ID NO:24, wherein the amino acids are replaced with glutamate or aspartate, and the second arm comprises a second human IgG1 constant region with amino acid substitutions at positions corresponding to positions 240 and 282 of SEQ ID NO:24, wherein the amino acids are replaced with lysine.

57. The bispecific agent of any one of claims 48-53, wherein the first arm comprises a first human IgG1 constant region with amino acid substitutions at positions corresponding to positions 240 and 282 of SEQ ID NO:24, wherein the amino acids are replaced with lysine, and the second arm comprises a second human IgG1 constant region with amino acid substitutions at positions corresponding to positions 253 and 292 of SEQ ID NO:24, wherein the amino acids are replaced with glutamate or aspartate.

58. The bispecific agent of any one of claims 48-53, wherein the first arm comprises a first human IgG4 constant region with amino acid substitutions at positions corresponding to positions 250 and 289 of SEQ ID NO:33 or SEQ ID NO:34, wherein the amino acids are replaced with glutamate or aspartate, and the second arm comprises a second human IgG4 constant region with amino acid substitutions at positions corresponding to positions 237 and 279 of SEQ ID NO:33 or SEQ ID NO:34, wherein the amino acids are replaced with lysine.

59. The bispecific agent of any one of claims 48-53, wherein the first arm comprises a first human IgG4 constant region with amino acid substitutions at positions corresponding to positions 237 and 279 of SEQ ID NO:33 or SEQ ID NO:34, wherein the amino acids are replaced with lysine, and the second arm comprises a second human IgG4 constant region with amino acid substitutions at positions corresponding to positions 250 and 289 of SEQ ID NO:33 or SEQ ID NO:34, wherein the amino acids are replaced with glutamate or aspartate.

60. The bispecific agent of claim 54, wherein the first and second CH3 domains are modified using a knobs-into-holes technique.

61. The bispecific agent of any one of claims 48-60, which inhibits binding of PD-L1 to PD-1.

62. The bispecific agent of any one of claims 48-60, which inhibits or blocks the interaction between PD-L1 and PD-1.

63. The bispecific agent of any one of claims 48-60, which inhibits binding of PD-L1 to CD80.

64. The bispecific agent of any one of claims 48-60, which inhibits or blocks the interaction between PD-L1 and CD80.

65. The bispecific agent of any one of claims 48-60, which inhibits PD-L1 signaling.

66. The bispecific agent of any one of claims 48-60, which is an antagonist of PD-L1-mediated signaling.

67. The bispecific agent of any one of claims 48-60, which inhibits PD-L1-mediated PD-1 activity.

68. The bispecific agent of any one of claims 48-60, which inhibits PD-L1-mediated CD80 activity.

69. The bispecific agent of any one of claims 48-60, which induces and/or enhances an immune response.

70. The bispecific agent of claim 69, wherein the immune response is directed to a tumor or tumor cell.

71. The bispecific agent of any one of claims 48-60, which increases cell-mediated immunity.

72. The bispecific agent of any one of claims 48-60, which increases T-cell activity.

73. The bispecific agent of any one of claims 48-60, which increases CTL activity.

74. The bispecific agent of any one of claims 48-60, which increases NK cell activity.

75. The bispecific agent of any one of claims 48-60, which increases IL-2 production and/or the number of IL-2-producing cells.

76. The bispecific agent of any one of claims 48-60, which increases IFN-gamma production and/or the number of IFN-gamma-producing cells.

77. The bispecific agent of any one of claims 48-60, which increases a Th1-type immune response.

78. The bispecific agent of any one of claims 48-60, which decreases IL-4 production and/or the number of IL-4-producing cells.

79. The bispecific agent of any one of claims 48-60, which decreases IL-10 and/or the number of IL-10-producing cells.

80. The bispecific agent of any one of claims 48-60, which decreases a Th2-type immune response.

81. The bispecific agent of any one of claims 48-60, which inhibits and/or decreases the suppressive activity of Tregs.

82. The bispecific agent of any one of claims 48-60, which inhibits and/or decreases the suppressive activity of MDSCs.

83. The bispecific agent of any one of claims 48-82, which inhibits tumor growth.

84. A polypeptide comprising a sequence selected from the group consisting of: SEQ ID NO:10, SEQ ID NO:11, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

85. A cell comprising or producing the antibody, bispecific agent, or polypeptide of any one of claims 1-84.

86. A composition comprising the antibody, bispecific agent, or polypeptide of any one of claims 1-84.

87. A pharmaceutical composition comprising the antibody, bispecific agent, or polypeptide of any one of claims 1-84 and a pharmaceutically acceptable carrier.

88. An isolated polynucleotide molecule comprising a polynucleotide that encodes an antibody, bispecific agent, or polypeptide of any one of claims 1-84.

89. An isolated polynucleotide comprising a polynucleotide sequence selected from the group consisting of: SEQ ID NO:12, SEQ ID NO:13, SEQ ID NO:22, SEQ ID NO:23, SEQ ID NO:24, SEQ ID NO:25, SEQ ID NO:26, SEQ ID NO:27, SEQ ID NO:28, and SEQ ID NO:29.

90. A vector comprising the polynucleotide of claim 88 or claim 89.

91. An isolated cell comprising the polynucleotide of claim 88 or claim 89.

92. An isolated cell comprising the vector of claim 91.

93. A method of inducing, activating, promoting, increasing, enhancing, or prolonging an immune response in a subject, comprising administering a therapeutically effective amount of the antibody, bispecific agent, or polypeptide of any one of claims 1-84.

94. A method of inducing, activating, promoting, increasing, enhancing, or prolonging an immune response in a subject, comprising administering a therapeutically effective amount of the antibody of any one of claims 1-23.

95. The method of claim 93 or claim 94, wherein the immune response is against a tumor or cancer.

96. A method of inhibiting growth of tumor cells, wherein the method comprises contacting the tumor cells with an effective amount of an antibody, bispecific agent, or polypeptide of any one of claims 1-84.

97. A method of inhibiting growth of a tumor in a subject, wherein the method comprises administering to the subject a therapeutically effective amount of an antibody, bispecific agent, or polypeptide of any one of claims 1-84.

98. A method of inhibiting growth of a tumor in a subject, wherein the method comprises administering to the subject a therapeutically effective amount of an antibody of any one of claims 1-23.

99. The method of any one of claims 95-98, wherein the tumor or tumor cell is selected from the group consisting of colorectal tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

100. A method of treating cancer in a subject, wherein the method comprises administering to the subject a therapeutically effective amount of an antibody, bispecific agent, or polypeptide of any one of claims 1-84.

101. A method of treating cancer in a subject, wherein the method comprises administering to the subject a therapeutically effective amount of an antibody of any one of claims 1-23.

102. The method of claim 100 or claim 101, wherein the cancer is selected from the group consisting of colorectal cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, and head and neck cancer.

103. The method of any one of claims 93-102, which further comprises administering at least one additional therapeutic agent.

104. The method of claim 103, wherein the additional therapeutic agent is a chemotherapeutic agent.

105. The method of claim 103, wherein the additional therapeutic agent is an antibody.

106. The method of claim 103, wherein the additional therapeutic agent is an immunotherapeutic agent.

107. The method of claim 106, wherein the immunotherapeutic agent is selected from the group consisting of: GM-CSF, M-CSF, G-CSF, IL-2, IL-3, IL-12, IL-15, B7-1 (CD80), B7-2 (CD86), 4-1BB ligand, GITRL, OX40 ligand, CD40 ligand, anti-CD3 antibody, anti-CTLA-4 antibody, anti-PD-1 antibody, anti-TIGIT antibody, anti-GITR antibody, anti-OX40 antibody, anti-CD40 antibody, anti-4-1BB antibody, anti-LAG-3 antibody, and anti-TIM-3 antibody.

108. The method of claim 103, wherein the additional therapeutic agent is an inhibitor of the Notch pathway, the Wnt pathway, or the RSPO/LGR pathway.

109. The method of any one of claim 93-95 or 97-108, wherein the subject has had a tumor or a cancer removed.

110. The method of any one of claims 95-109, wherein the tumor or the cancer expresses PD-L1.

111. The method of any one of claims 95-110, further comprising a step of determining the level of PD-L1 expression in the tumor or cancer.

112. The method of claim 111, wherein determining the level of PD-L1 expression is done prior to treatment or contact with the antibody.

113. A plasmid deposited with ATCC and assigned designation number PTA-122627.

114. A plasmid deposited with ATCC and assigned designation number PTA-122628.


SEQUENCE LISTING (34 sequences)

SEQ ID NO: 1 - protein, 290 aa, Artificial Sequence
  Synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 272 aa, Artificial Sequence
  Synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 3 - protein, 223 aa, Artificial Sequence
  Synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHL

SEQ ID NO: 4 - protein, 6 aa, Artificial Sequence
  Synthetic construct
TSYWMH

SEQ ID NO: 5 - protein, 17 aa, Artificial Sequence
  Synthetic construct
AIYPGNSDTSYNQKFKG

SEQ ID NO: 6 - protein, 11 aa, Artificial Sequence
  Synthetic construct
WGYGFDGAMDY

SEQ ID NO: 7 - protein, 11 aa, Artificial Sequence
  Synthetic construct
RASQDIGSSLN

SEQ ID NO: 8 - protein, 7 aa, Artificial Sequence
  Synthetic construct
ATSSLDS

SEQ ID NO: 9 - protein, 7 aa, Artificial Sequence
  Synthetic construct
LQYASSP

SEQ ID NO: 10 - protein, 120 aa, Artificial Sequence
  Synthetic construct
QVQLQQSGPELARPGASVKMSCKASGYSFTSYWMHWVKQRPGQGLEWIGAIYPGNSDTSYNQKFKGKAKLTAVTSASTAYMELSSLTNEDSAVYYCTRWGYGFDGAMDYWGQGTSVTVSS

SEQ ID NO: 11 - protein, 107 aa, Artificial Sequence
  Synthetic construct
DIVTQSPSSLSASLGERVSLTCRASQDIGSSLNWLQQEPDGTIKRLIYATSSLDSGVPKRFSGSRSGSDYSLTISSLESEDFVDYYCLQYASSPYTFGGGTKLEIKR

SEQ ID NO: 12 - DNA, 360 bp, Artificial Sequence
  Synthetic construct
caagtccaattgcagcagtctggacctgagctggcaaggcctggggcttccgtgaagatgtcctgcaaggcttctggctacagctttaccagctactggatgcactgggtaaaacagaggcctggacagggtctagaatggattggtgctatttatcctggaaatagtgatactagctacaaccagaagttcaagggcaaggccaagctgactgcagtcacatccgccagcactgcctacatggagctcagcagcctgacaaatgaggactctgcggtctattactgtacaagatgggggtatgggttcgacggagctatggactactggggtcaaggaacctcagtcaccgtctcctca

SEQ ID NO: 13 - DNA, 321 bp, Artificial Sequence
  Synthetic construct
gatatcgtgacccagtctccatcctccttatctgcctctctgggagaaagagtcagtctcacttgtcgggcaagtcaggacattggtagtagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatatgctagttctccgtacacgttcggaggggggaccaagctggaaataaaacgg

SEQ ID NO: 14 - protein, 120 aa, Artificial Sequence
  Synthetic construct
QVQLVQSGAEVKKPGASVKVSCKASGYTFTSYWMHWVRQAPGQGLEWMGAIYPGNSDTSYNQKFKGRVTMTRDTSTSTVYMELSSLRSEDTAVYYCTRWGYGFDGAMDYWGQGTLVTVSS

SEQ ID NO: 15 - protein, 108 aa, Artificial Sequence
  Synthetic construct
DIQMTQSPSSLSASVGDRVTITCRASQDIGSSLNWYQQKPGKAPKRLIYATSSLDSGVPSRFSGSGSGTEFTLTISSLQPEDFATYYCLQYASSPYTFGGGTKVEIKR

SEQ ID NO: 16 - protein, 469 aa, Artificial Sequence
  Synthetic construct
MDWTWRILFLVAAATGAHSQVQLVQSGAEVKKPGASVKVSCKASGYTFTSYWMHWVRQAPGQGLEWMGAIYPGNSDTSYNQKFKGRVTMTRDTSTSTVYMELSSLRSEDTAVYYCTRWGYGFDGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 450 aa, Artificial Sequence
  Synthetic construct
QVQLVQSGAEVKKPGASVKVSCKASGYTFTSYWMHWVRQAPGQGLEWMGAIYPGNSDTSYNQKFKGRVTMTRDTSTSTVYMELSSLRSEDTAVYYCTRWGYGFDGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 466 aa, Artificial Sequence
  Synthetic construct
MDWTWRILFLVAAATGAHSQVQLVQSGAEVKKPGASVKVSCKASGYTFTSYWMHWVRQAPGQGLEWMGAIYPGNSDTSYNQKFKGRVTMTRDTSTSTVYMELSSLRSEDTAVYYCTRWGYGFDGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 447 aa, Artificial Sequence
  Synthetic construct
QVQLVQSGAEVKKPGASVKVSCKASGYTFTSYWMHWVRQAPGQGLEWMGAIYPGNSDTSYNQKFKGRVTMTRDTSTSTVYMELSSLRSEDTAVYYCTRWGYGFDGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 234 aa, Artificial Sequence
  Synthetic construct
MVLQTQVFISLLLWISGAYGDIQMTQSPSSLSASVGDRVTITCRASQDIGSSLNWYQQKPGKAPKRLIYATSSLDSGVPSRFSGSGSGTEFTLTISSLQPEDFATYYCLQYASSPYTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 21 - protein, 214 aa, Artificial Sequence
  Synthetic construct
DIQMTQSPSSLSASVGDRVTITCRASQDIGSSLNWYQQKPGKAPKRLIYATSSLDSGVPSRFSGSGSGTEFTLTISSLQPEDFATYYCLQYASSPYTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 22 - DNA, 324 bp, Artificial Sequence
  Synthetic construct
gacatccagatgacccagtctccatcctccctgtctgcatctgtcggagacagagtcaccatcacttgccgggcatctcaggacattggttcctctctcaactggtatcagcagaaaccagggaaagcccctaagcgcctgatctatgccacatcctctctggattctggggtcccatcaaggttcagcggctccggatctgggacagaatttactctcacaatcagcagcctgcagcctgaagattttgcaacttattactgtctccaatatgcttcttctccttacactttcggcggagggaccaaggtggagatcaaacgt

SEQ ID NO: 23 - DNA, 324 bp, Artificial Sequence; identical to SEQ ID NO: 22
  Synthetic construct

SEQ ID NO: 24 - DNA, 1410 bp, Artificial Sequence
  Synthetic construct
atggactggacctggaggatactctttctcgtggctgcagccacaggagcccactcccaagtccagctggtgcagtctggggctgaggtgaagaagcctggggcctcagtgaaggtttcctgcaaggcatctggatacaccttcaccagctactggatgcactgggtgcggcaggcccctggacaagggctcgaatggatgggagctatttatcctggaaattccgatactagctacaaccagaagttcaagggcagagtcaccatgaccagggacacatccactagcacagtctacatggagctgtctagcctgcggtctgaggacactgccgtgtattactgtacaagatgggggtatgggttcgacggagctatggactactggggccagggcaccctggtcaccgtcagctcagccagcacaaagggcccctccgtgttccctctggccccttcctccaagtccacctccggcggcaccgccgctctgggctgcctggtgaaggactacttccctgagcctgtgaccgtgtcctggaactctggcgccctgacctctggcgtgcacaccttcccagccgtgctgcagtcctccggcctgtactccctgtcctccgtggtgaccgtgccttcctcctccctgggcacccagacctacatctgcaacgtgaaccacaagccttccaacaccaaggtggacaagcgggtggagcctaagtcctgcgacaagacccacacctgccctccctgccctgcccctgagctgctgggcggaccttccgtgttcctgttccctcctaagcctaaggacaccctgatgatctcccggacccctgaggtgacctgcgtggtggtggacgtgtcccacgaggatcctgaggtgaagttcaattggtacgtggacggcgtggaggtgcacaacgctaagaccaagccaagggaggagcagtacaactccacctaccgggtggtgtctgtgctgaccgtgctgcaccaggactggctgaacggcaaagaatacaagtgcaaggtctccaacaaggccctgcccgctcccatcgagaaaaccatctccaaggccaagggccagcctcgcgagcctcaggtgtacaccctgccacccagccgggaggagatgaccaagaaccaggtgtccctgacctgtctggtgaagggcttctacccttccgatatcgccgtggagtgggagtctaacggccagcccgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagagcctgtctctgtctcctggcaagtga

SEQ ID NO: 25 - DNA, 1353 bp, Artificial Sequence
  Synthetic construct
caagtccagctggtgcagtctggggctgaggtgaagaagcctggggcctcagtgaaggtttcctgcaaggcatctggatacaccttcaccagctactggatgcactgggtgcggcaggcccctggacaagggctcgaatggatgggagctatttatcctggaaattccgatactagctacaaccagaagttcaagggcagagtcaccatgaccagggacacatccactagcacagtctacatggagctgtctagcctgcggtctgaggacactgccgtgtattactgtacaagatgggggtatgggttcgacggagctatggactactggggccagggcaccctggtcaccgtcagctcagccagcacaaagggcccctccgtgttccctctggccccttcctccaagtccacctccggcggcaccgccgctctgggctgcctggtgaaggactacttccctgagcctgtgaccgtgtcctggaactctggcgccctgacctctggcgtgcacaccttcccagccgtgctgcagtcctccggcctgtactccctgtcctccgtggtgaccgtgccttcctcctccctgggcacccagacctacatctgcaacgtgaaccacaagccttccaacaccaaggtggacaagcgggtggagcctaagtcctgcgacaagacccacacctgccctccctgccctgcccctgagctgctgggcggaccttccgtgttcctgttccctcctaagcctaaggacaccctgatgatctcccggacccctgaggtgacctgcgtggtggtggacgtgtcccacgaggatcctgaggtgaagttcaattggtacgtggacggcgtggaggtgcacaacgctaagaccaagccaagggaggagcagtacaactccacctaccgggtggtgtctgtgctgaccgtgctgcaccaggactggctgaacggcaaagaatacaagtgcaaggtctccaacaaggccctgcccgctcccatcgagaaaaccatctccaaggccaagggccagcctcgcgagcctcaggtgtacaccctgccacccagccgggaggagatgaccaagaaccaggtgtccctgacctgtctggtgaagggcttctacccttccgatatcgccgtggagtgggagtctaacggccagcccgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagagcctgtctctgtctcctggcaagtga

SEQ ID NO: 26 - DNA, 1401 bp, Artificial Sequence
  Synthetic construct
atggactggacctggaggatactctttctcgtggctgcagccacaggagcccactcccaagtccagctggtgcagtctggggctgaggtgaagaagcctggggcctcagtgaaggtttcctgcaaggcatctggatacaccttcaccagctactggatgcactgggtgcggcaggcccctggacaagggctcgaatggatgggagctatttatcctggaaattccgatactagctacaaccagaagttcaagggcagagtcaccatgaccagggacacatccactagcacagtctacatggagctgtctagcctgcggtctgaggacactgccgtgtattactgtacaagatgggggtatgggttcgacggagctatggactactggggccagggcaccctggtcaccgtcagctcagccagcacaaagggcccatccgtcttccccctggcaccctgctcccggagcacctccgagagcacagccgccctgggctgcctggtcaaggactacttccccgaacccgttaccgtgtcttggaactccggcgcactgaccagcggcgtgcacaccttccctgctgtcctccaatcctctggactctactccctctcctccgtggtgacagtgccctccagcagcctgggcactaagacctacacctgcaacgtcgatcacaagcccagcaacaccaaggtggacaagagagttgagtccaaatatggacccccatgcccaccttgcccagcacctgagttcctggggggaccatcagtcttcctgttccccccaaaacccaaggacactctcatgatctcccggacccctgaggtcacttgcgtggtggtggacgtgagccaggaagaccccgaggtccagttcaactggtatgtggatggcgtggaggttcataatgccaagacaaagcctcgggaggagcagttcaacagcacctaccgggtggtcagcgtcctcaccgtcctgcaccaagactggctgaacggcaaggagtacaagtgcaaggtctccaacaaagggctcccatcctccatcgagaaaaccatctccaaagccaaagggcagccccgggagccacaggtgtacaccctgcccccatcccaagaggagatgaccaagaaccaagtgtccctgacctgcctggtcaaaggcttctaccccagcgacatcgccgtggagtgggagagcaatgggcagcctgagaacaactacaagaccactcctcccgtgctggactccgacggctccttcttcctctactcccggctcaccgtggacaagagcaggtggcaggagggcaatgtcttctcctgctccgtgatgcatgaggctctgcacaaccactacacacagaagagcctctccctgtctctgggcaaatga

SEQ ID NO: 27 - DNA, 1344 bp, Artificial Sequence
  Synthetic construct
caagtccagctggtgcagtctggggctgaggtgaagaagcctggggcctcagtgaaggtttcctgcaaggcatctggatacaccttcaccagctactggatgcactgggtgcggcaggcccctggacaagggctcgaatggatgggagctatttatcctggaaattccgatactagctacaaccagaagttcaagggcagagtcaccatgaccagggacacatccactagcacagtctacatggagctgtctagcctgcggtctgaggacactgccgtgtattactgtacaagatgggggtatgggttcgacggagctatggactactggggccagggcaccctggtcaccgtcagctcagccagcacaaagggcccatccgtcttccccctggcaccctgctcccggagcacctccgagagcacagccgccctgggctgcctggtcaaggactacttccccgaacccgttaccgtgtcttggaactccggcgcactgaccagcggcgtgcacaccttccctgctgtcctccaatcctctggactctactccctctcctccgtggtgacagtgccctccagcagcctgggcactaagacctacacctgcaacgtcgatcacaagcccagcaacaccaaggtggacaagagagttgagtccaaatatggacccccatgcccaccttgcccagcacctgagttcctggggggaccatcagtcttcctgttccccccaaaacccaaggacactctcatgatctcccggacccctgaggtcacttgcgtggtggtggacgtgagccaggaagaccccgaggtccagttcaactggtatgtggatggcgtggaggttcataatgccaagacaaagcctcgggaggagcagttcaacagcacctaccgggtggtcagcgtcctcaccgtcctgcaccaagactggctgaacggcaaggagtacaagtgcaaggtctccaacaaagggctcccatcctccatcgagaaaaccatctccaaagccaaagggcagccccgggagccacaggtgtacaccctgcccccatcccaagaggagatgaccaagaaccaagtgtccctgacctgcctggtcaaaggcttctaccccagcgacatcgccgtggagtgggagagcaatgggcagcctgagaacaactacaagaccactcctcccgtgctggactccgacggctccttcttcctctactcccggctcaccgtggacaagagcaggtggcaggagggcaatgtcttctcctgctccgtgatgcatgaggctctgcacaaccactacacacagaagagcctctccctgtctctgggcaaatga

SEQ ID NO: 28 - DNA, 702 bp, Artificial Sequence
  Synthetic construct
atggactggacctggaggatactctttctcgtggctgcagccacaggagcccactccgacatccagatgacccagtctccatcctccctgtctgcatctgtcggagacagagtcaccatcacttgccgggcatctcaggacattggttcctctctcaactggtatcagcagaaaccagggaaagcccctaagcgcctgatctatgccacatcctctctggattctggggtcccatcaaggttcagcggctccggatctgggacagaatttactctcacaatcagcagcctgcagcctgaagattttgcaacttattactgtctccaatatgcttcttctccttacactttcggcggagggaccaaggtggagatcaaacgtacggtggctgcaccatctgtcttcatcttccctccatctgatgagcagctcaaatctggaactgcctctgttgtgtgcctgctgaataacttctatcccagagaggccaaagtccagtggaaggtggataacgccctccaatccggcaactcccaggagagtgtcacagagcaggacagcaaggacagcacctacagcctcagcaacaccctgacactgagcaaagcagactacgagaaacacaaagtctatgcctgcgaagtcacccatcagggcctgtcttcccccgtcacaaagagcttcaacaggggagagtgctaa

SEQ ID NO: 29 - DNA, 642 bp, Artificial Sequence
  Synthetic construct
atccagatgacccagtctccatcctccctgtctgcatctgtcggagacagagtcaccatcacttgccgggcatctcaggacattggttcctctctcaactggtatcagcagaaaccagggaaagcccctaagcgcctgatctatgccacatcctctctggattctggggtcccatcaaggttcagcggctccggatctgggacagaatttactctcacaatcagcagcctgcagcctgaagattttgcaacttattactgtctccaatatgcttcttctccttacactttcggcggagggaccaaggtggagatcaaacgtacggtggctgcaccatctgtcttcatcttccctccatctgatgagcagctcaaatctggaactgcctctgttgtgtgcctgctgaataacttctatcccagagaggccaaagtccagtggaaggtggataacgccctccaatccggcaactcccaggagagtgtcacagagcaggacagcaaggacagcacctacagcctcagcaacaccctgacactgagcaaagcagactacgagaaacacaaagtctatgcctgcgaagtcacccatcagggcctgtcttcccccgtcacaaagagcttcaacaggggagagtgctaa

SEQ ID NO: 30 - protein, 330 aa, Artificial Sequence [sequence omitted]

SEQ ID NO: 31 - protein, 326 aa, Artificial Sequence
  Synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 377 aa, Artificial Sequence
  Synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 33 - protein, 327 aa, Artificial Sequence
  Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 327 aa, Artificial Sequence
  Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK